(19) United States
(12) Patent Application Publication
(10) Pub. No.: US 2024/0091487 A1
(43) Pub. Date: Mar. 21, 2024

(54) BED SYSTEM FOR ADJUSTING A SLEEP ENVIRONMENT BASED ON MICROCLIMATE TEMPERATURE AND SLEEP QUALITY OPTIMIZATION

(71) Applicant: Sleep Number Corporation, Minneapolis, MN (US)

(72) Inventors: Gary N. Garcia Molina, Madison, WI (US); Cristina Marie Jocson, San Francisco, CA (US); Jessie Jiang, San Jose, CA (US); Pavlo Chernega, Kyiv (UA); Megha Rajam Rao, San Jose, CA (US); Nikhil Makaram, Santa Clara, CA (US)

(21) Appl. No.: 18/370,552

(22) Filed: Sep. 20, 2023

Related U.S. Application Data
(60) Provisional application No. 63/408,241, filed on Sep. 20, 2022.

(51) Int. Cl. A61M 21/00; A61B 5/00

(52) U.S. Cl. CPC A61M 21/00; A61B 5/4815; A47C 31/008; A61M 2021/0066; A61M 2205/3368; A61M 2205/50

(57) ABSTRACT

The disclosed technology provides for improving sleep quality of a user by adjusting a sleep environment. A bed system can include, for example, a controller that can retrieve, from a data repository, data for a set of past sleep sessions of a user, identify, based on the data, a past sleep session in the set of past sleep sessions that satisfies sleep quality criteria, determine thermal settings for the bed system based on the data associated with the identified past sleep session of the user, and apply the thermal settings to the bed system for execution during at least one subsequent sleep session of the user.

### CROSS-REFERENCE TO RELATED APPLICATIONS

This application claims the benefit of U.S. Provisional Application Ser. No. 63/408,241, filed Sep. 20, 2022. The disclosure of the prior application is considered part of the disclosure of this application, and is incorporated in its entirety into this application.

The present document relates to systems and techniques for determining thermal routines of a bed system based on automatically processing data such as microclimate temperature data of the bed system and sleep quality data from sleep sessions of a user of the bed system.

### BACKGROUND

In general, a bed is a piece of furniture used as a location to sleep or relax. Many modern beds include a soft mattress on a bed frame. The mattress may include springs, foam material, and/or an air chamber to support the weight of one or more occupants.

### SUMMARY

This document generally relates to systems and techniques for determining thermal routines for a bed system that improve sleep quality of a user of the bed system. More particularly, the disclosed technology provides for processing microclimate temperature data collected at the bed system during past sleep sessions of the user to determine thermal routines that can be implemented at the bed system during subsequent sleep sessions. Processing the microclimate temperature data can include identifying a past sleep session during which the user experienced a threshold level of sleep quality and/or a highest level of sleep quality. Processing the microclimate temperature data can also include mapping the microclimate temperature data of the identified past sleep session to one or more thermal settings, which can then be implemented during subsequent sleep sessions of the user. The thermal settings can be implemented during the subsequent sleep sessions to essentially copy the microclimate of the identified past sleep session and help the user achieve an improved level of sleep quality during the subsequent sleep sessions.

The microclimate temperature data can correspond to a temperature of air surrounding the user as they are resting on a top surface of the bed system. The microclimate temperature data can also correspond to a temperature at the top surface of the bed system where the user rests. Some patterns of microclimate temperature data can promote the user's sleep quality by influencing the dynamics of core body temperature (CBT) of the user such that CBT decreases while falling asleep, remains low during the first two sleep cycles (e.g., ˜5 hours from sleep onset)—and increases in the final cycle of sleep. Therefore, the disclosed technology provides techniques to control the microclimate temperature data to improve or otherwise maintain threshold levels of sleep quality of the user during sleep sessions.

Some embodiments described herein include a bed system for improving sleep quality of a user by adjusting a sleep environment, the bed system can include: a controller that can be configured to: retrieve, from a data repository, data for a set of past sleep sessions of a user, identify, based on the data, a past sleep session in the set of past sleep sessions that satisfies sleep quality criteria, determine thermal settings for the bed system based on the data associated with the identified past sleep session of the user, and apply the thermal settings to the bed system for execution during at least one subsequent sleep session of the user.

Embodiments described herein can include one or more optional features. For example, the set of past sleep sessions can include multiple past sleep sessions of the user. Identifying the past sleep session in the set can include determining that a sleep quality value in the data for the identified sleep session is greater than sleep quality values in the data for other past sleep sessions in the set. Identifying the past sleep session in the set can also include determining that a sleep quality value in the data for the identified past sleep session exceeds a threshold sleep quality value. The data for the set of past sleep sessions may include a temperature profile for each of the past sleep sessions, in which the temperature profile can be a time sequence of temperature values collected by temperature sensors at a top surface of the bed system of the user throughout the past sleep session. Determining the thermal settings for the bed system may include applying a model to a temperature profile of the identified past sleep session, the model having been trained to identify segments of temperature values in the temperature profile and identify relationships between each of the segments of temperature values and one or more predetermined thermal settings.

As another example, applying the thermal settings to the bed system can include generating instructions to activate a thermal routine at a heating or cooling unit of the bed system during the at least one subsequent sleep session of the user to adjust a microclimate at a top surface of the bed system to at least one temperature value in the data for the identified past sleep session. Determining the thermal settings for the bed system may include determining the thermal settings for each segment of the identified past sleep session. Each segment of the identified past sleep session can be a 60-minute period of time, in which the segments may be non-overlapping periods of time during the identified past sleep session. Identifying, based on the data, the past sleep session in the set of past sleep sessions that satisfies sleep quality criteria can include identifying the past sleep session in the set having a sleep quality metric in the data for the past sleep session that exceeds a threshold sleep quality value. The sleep quality metric in the data for the past sleep session can be a numeric value indicating a sleep quality score and the threshold sleep quality value can be 90. Identifying, based on the data, the past sleep session in the set of past sleep sessions that satisfies sleep quality criteria may include: ranking the past sleep sessions in the set from highest sleep quality metric to lowest sleep quality metric, in which the data for each of the past sleep sessions in the set may include a sleep quality metric corresponding to the past sleep session, and selecting the past sleep session that is ranked the highest among the ranked past sleep sessions.

Some embodiments described herein include a bed system for improving sleep quality of a user by adjusting a sleep environment, the bed system including: a controller that can be configured to: receive, from at least one temperature sensor of the bed system, temperature data collected during a sleep session of a user of the bed system, determine a microclimate temperature value for each predetermined interval of time during the sleep session based on applying a model to the temperature data, determine a microclimate temperature value for the sleep session based on aggregating the microclimate temperature values for the predetermined intervals of time during the sleep session, and return at least one of (i) the microclimate temperature values for the predetermined intervals of time during the sleep session or (ii) the microclimate temperature value for the sleep session.

The bed system can optionally include one or more of the following features. For example, the at least one temperature sensor can be configured to a sensor strip, the sensor strip removably attached to a top surface of the bed system on which the user rests. The at least one temperature sensor can include 5 temperature sensors linearly arranged along the sensor strip, in which the 5 temperature sensors may be equally spaced apart by a threshold distance along the sensor strip. The bed system can be a queen-sized bed and the threshold distance can be 5.5 inches. The bed system can be a king-sized bed and the threshold distance can be 6.5 inches. The model can be trained to approximate, based on the temperature data, a microclimate temperature value for each of the predetermined time intervals during the sleep session. The predetermined time intervals can be one-minute segments during the sleep session.

Some embodiments described herein include a bed system for improving sleep quality of a user by adjusting a sleep environment, the bed system including: a controller that can be configured to: receive temperature profiles of past sleep sessions of a user of the bed system, determine thermal settings that replicate a temperature profile of at least one of the past sleep sessions based on applying a model to the temperature profile of the at least one of the past sleep sessions, and return the thermal settings.

The bed system can optionally include one or more of the following features. For example, the temperature profile of the at least one of the past sleep sessions can include multiple temperature values detected at a top surface of the bed system throughout the at least one of the past sleep sessions. The controller can be configured to determine the thermal settings for each segment of the at least one of the past sleep sessions, in which each segment of the sleep session can be a non-overlapping 60-minute period of time. The controller can be configured to determine the thermal settings for a continuous period of time that the user is on the bed system during the at least one of the past sleep sessions. The controller can further be configured to program a thermal routine of a heating or cooling unit of the bed system to execute the returned thermal settings during at least one subsequent sleep session of the user. Each of the received temperature profiles may include a time sequence of temperature values collected by temperature sensors of the bed system during the past sleep sessions when one or more different thermal settings are activated at the bed system.

Determining the thermal settings may include: identifying one or more thermal condition parameters of a microclimate at a top surface of the bed system based on fitting the model to each of the temperature profiles, in which the thermal condition parameters indicate effects of one or more different thermal settings on the microclimate at the top surface of the bed system, and determining the thermal settings based on the identified thermal condition parameters. The controller can also: receive temperature profiles of past sleep sessions of a population of users that includes the user, select a temperature profile amongst the temperature profiles for the population of users that satisfies threshold thermal settings criteria, and determine the thermal settings that replicate the selected temperature profile based on identifying relationships between temperature values of the selected temperature profile and one or more predetermined thermal settings for a heating or cooling unit of the bed system. Sometimes, determining the thermal settings that replicate the temperature profile of the at least one of the past sleep sessions may include: retrieving, from a data repository, a library of thermal settings that map each of the retrieved thermal settings with effects of the retrieved thermal settings on a microclimate at a top surface of the bed system, and identifying, based on the effects of the retrieved thermal settings on the microclimate, one of the retrieved thermal settings that causes the microclimate at the top surface of the bed system to achieve at least one temperature value in the temperature profile of the at least one of the past sleep sessions.

Some embodiments described herein include a bed system for improving sleep quality of a user by adjusting a sleep environment, the bed system including: a controller that can be configured to: retrieve, from a data repository, data for a set of past sleep sessions of a user, identify, based on the data, a past sleep session in the set of past sleep sessions that satisfies sleep quality criteria, identify thermal settings for execution during at least one subsequent sleep session of the user based on the data associated with the identified past sleep session, and return the thermal settings.

The bed system can optionally include one or more of the following features. For example, the sleep quality criteria can indicate temperature profile-data that results in the user experiencing a threshold level of sleep quality during the past sleep session. Identifying the thermal settings can include applying a model to the identified past sleep session, the model having been trained to identify segments of temperature values in a temperature profile in the data for the identified past sleep session during the past sleep session and identify relationships between each of the segments of temperature values and one or more predetermined thermal settings. Identifying the thermal settings can include: retrieving, from a data repository, mappings of temperature values in the data for the set of past sleep sessions to one or more predefined thermal settings, the predefined thermal settings including high heat, medium heat, low heat, high cool, medium cool, low cool, and off, and selecting, based on the mappings, at least one of the predefined thermal settings that corresponds to temperature values in a temperature profile in the data for the identified past sleep session. Returning the thermal settings can include storing, in a data repository, the thermal settings for future use, by the controller, to determine when to activate a heating or cooling unit of the bed system during the at least one subsequent sleep session of the user. The controller can also program a thermal routine of a heating or cooling unit of the bed system to execute the returned thermal settings during the at least one subsequent sleep session of the user.

Some embodiments described herein include a bed system for improving sleep quality of a user by adjusting a sleep environment, the bed system including: a controller that can be configured to: retrieve, from a data repository, temperature profiles corresponding to a threshold quantity of past sleep sessions of a user, in which each temperature profile is a time sequence of temperature values collected by temperature sensors at a top surface of a bed of the user throughout a sleep session of the user, identify a temperature profile amongst the retrieved temperature profiles that satisfies threshold thermal settings criteria, in which the threshold thermal settings criteria can indicate temperature profile-data that results in the user experiencing a threshold level of sleep quality during the sleep session, process the identified temperature profile to identify relationships between the temperature values of the identified temperature profile and thermal settings for a heating or cooling unit of the bed, in which executing, by the heating or cooling unit of the bed, the thermal settings during a subsequent sleep session of the user causes a microclimate at the top surface of the bed to be adjusted, by the heating or cooling unit of the bed, to the temperature values of the identified temperature profile to cause the user to experience the threshold level of sleep quality during the subsequent sleep session, and return the thermal settings.

The bed system can optionally include one or more of the following features. For example, processing the identified temperature profile can include applying a model to the identified temperature profile, the model having been trained to identify segments of the temperature values in the temperature profile throughout the sleep session of the user and identify relationships between each of the segments of temperature values and one or more predetermined thermal settings. Returning the thermal settings can include generating instructions to activate a thermal routine at the heating or cooling unit of the bed during the subsequent sleep session of the user to adjust the microclimate at the top surface of the bed to at least one of the temperature values of the identified temperature profile. During the subsequent sleep session, the controller can be configured to: receive, from the temperature sensors, real-time temperature values detected at the top surface of the bed while the user is on the bed, determine whether the real-time temperature values satisfy threshold thermal-routine-activation criteria, and activate the thermal routine based on a determination that the threshold thermal-routine-activation criteria is satisfied.

Sometimes, activating the thermal routine can include turning on a heating element of the heating or cooling unit to a setting defined by the thermal settings until the at least one temperature value in the identified temperature profile is detected, by the temperature sensors, at the top surface of the bed. Activating the thermal routine can also include turning off a heating element of the heating or cooling unit while the at least one temperature value in the identified temperature profile is detected, by the temperature sensors, at the top surface of the bed. Activating the thermal routine can include turning on a cooling element of the heating or cooling unit until the at least one temperature value in the identified temperature profile is detected, by the temperature sensors, at the top surface of the bed. Activating the thermal routine can include turning off a cooling element of the heating or cooling unit while the at least one temperature value in the identified temperature profile is detected, by the temperature sensors, at the top surface of the bed. Moreover, determining whether the real-time temperature values satisfy the threshold thermal-routine-activation criteria can include determining that an average of the real-time temperature values exceeds a threshold average temperature value for the identified temperature profile. Determining whether the real-time temperature values satisfy threshold thermal-routine-activation criteria can also include determining that an average of the real-time temperature values is less than a threshold average temperature value for the identified temperature profile. In some implementations, the subsequent sleep session of the user can include one or more segments, the threshold thermal-routine-activation criteria can be different for each segment of the sleep session, and the controller can activate the thermal routine during each segment of the subsequent sleep session based on a determination of whether the respective threshold thermal-routine-activation criteria for the segment is satisfied.

In some implementations, identifying the temperature profile amongst the retrieved temperature profiles can include: retrieving, from the data repository and for each sleep session in the threshold quantity of past sleep sessions, a sleep quality metric, identifying a sleep session amongst the threshold quantity of past sleep sessions having the sleep quality metric that exceeds a threshold sleep quality value, and selecting the temperature profile that corresponds to the identified sleep session. The sleep quality metric can be a numeric value indicating a sleep quality score. The sleep quality metric can be a user-perceived sleep quality value. The sleep quality metric can be a numeric value and the threshold sleep quality value can be 90.

As another example, identifying the temperature profile amongst the retrieved temperature profiles can include: retrieving, from the data repository and for each sleep session in the threshold quantity of past sleep sessions, a sleep quality metric, identifying a sleep session amongst the threshold quantity of past sleep sessions having a highest sleep quality metric amongst the sleep quality metrics for the threshold quantity of past sleep sessions, and selecting the temperature profile that corresponds to the identified sleep session. Identifying the temperature profile amongst the retrieved temperature profiles can also include: ranking the retrieved temperature profiles based on respective sleep quality metrics and selecting a highest ranked temperature profile amongst the ranked temperature profiles. The retrieved temperature profiles can be ranked from highest sleep quality metric to lowest sleep quality metric. The threshold quantity of past sleep sessions of the user can be between 7 and 8 successive sleep sessions. The threshold thermal settings criteria can change based on a current season, in which the current season includes at least one of winter, spring, summer, or fall. The threshold thermal settings criteria can change based on a circadian rhythm of the user. The threshold thermal settings criteria can change based on an age of the user. The threshold thermal settings criteria can also change based on a day of a week.

As another example, returning the thermal settings can include transmitting, to a computing device of the user, instructions that cause the computing device to present, in a graphical user interface (GUI) display, a user-selectable option to select the thermal settings to be executed, by the heating or cooling unit of the bed, as part of a thermal routine during the subsequent sleep session of the user. The controller may also be configured to: detect, based on first pressure values collected by at least one sensor of the bed, presence of the user on a first side of the bed, detect, based on second pressure values collected by the at least one sensor of the bed, presence of a second user on a second side of the bed, receive, from the temperature sensors, temperature values detected at the top surface of the bed when the user and the second user are detected on the bed, generate respective temperature profiles for the user and the second user based on applying a temperature model to the received temperature values, the temperature model having been trained to differentiate the received temperature values for respective sides of the bed and generate a temperature profile for each side of the bed, and store, in the data repository, the respective temperature profiles for the user and the second user for later use, by the controller, in determining thermal settings for each side of the bed during subsequent sleep sessions of the respective user and second user.

Some embodiments described herein can include a bed system for improving sleep quality of a user by adjusting a sleep environment, the bed system including: a controller that can be configured to: retrieve, from a data repository, temperature profiles corresponding to a threshold quantity of past sleep sessions of a user, in which each temperature profile is a time sequence of temperature values collected by temperature sensors at a top surface of a bed of the user throughout a sleep session of the user, identify a temperature profile amongst the retrieved temperature profiles that satisfies threshold thermal settings criteria, in which the threshold thermal settings criteria indicate temperature profile-data that results in the user experiencing a threshold level of sleep quality during the sleep session, identify thermal settings for a heating or cooling unit of the bed based on applying a model to the identified temperature profile, the model having been trained to identify segments of the temperature values in the identified temperature profile and identify relationships between each of the segments and one or more predetermined thermal settings, and return the thermal settings for execution by the heating or cooling unit of the bed, in which executing the thermal settings during a subsequent sleep session of the user causes a microclimate at the top surface of the bed to be automatically adjusted, by the heating or cooling unit of the bed, to one or more of the temperature values of the identified temperature profile to cause the user to experience the threshold level of sleep quality during the subsequent sleep session.

The bed system can optionally include one or more of the abovementioned features.

Some embodiments described herein include a bed system for improving sleep quality of a user by adjusting a sleep environment, the bed system including: a controller that can be configured to: retrieve, from a data repository, temperature profiles corresponding to a threshold quantity of past sleep sessions of a user, in which each temperature profile can be a time sequence of temperature values collected by temperature sensors at a top surface of a bed of the user throughout a sleep session of the user, retrieve, from the data repository and for each sleep session, a corresponding sleep quality metric, identify a target sleep session for replicating a microclimate at the top surface of the bed by identifying one of the threshold quantity of past sleep sessions having the corresponding sleep quality metric that exceeds a threshold sleep quality value, select a temperature profile amongst the retrieved temperature profiles that corresponds to the identified target sleep session, determine thermal settings that correspond to the temperature values of the selected temperature profile, in which the thermal settings include a heating or cooling routine that, when activated by a component of the bed, causes the microclimate at the top surface of the bed to be adjusted to the microclimate of the identified target sleep session, and return the thermal settings.

The bed system can optionally include one or more of the abovementioned features and/or one or more of the following features. For example, the sleep quality metric can be a numeric value indicating an average level of sleep quality experienced by the user during the identified target sleep session of the user. The threshold sleep quality value can be 90. Identifying the target sleep session can include: ranking the threshold quantity of past sleep sessions from highest corresponding sleep quality metric to lowest corresponding sleep quality metric, and identifying the target sleep session based on selecting a top-ranked sleep session amongst the ranked past sleep sessions.

Some embodiments described herein include a system for determining and applying thermal settings for subsequent sleep sessions based on a past sleep session that satisfies sleep quality criteria. The system can optionally include one or more of the abovementioned features.

Some embodiments described herein include a system for identifying thermal settings for subsequent sleep sessions based on temperature data for a past sleep session that satisfies sleep quality criteria. The system can optionally include one or more of the abovementioned features.

Some embodiments described herein include a system for modeling temperature sensor readings to determine interval-based microclimate temperatures and an aggregate microclimate temperature for an entire sleep session. The system can optionally include one or more of the abovementioned features.

Some embodiments described herein include a system for modeling temperature profiles of past sleep sessions to determine thermal settings that replicate microclimates of the past sleep sessions. The system can optionally include one or more of the abovementioned features. Moreover, the thermal settings can be generated by the system in response to the modeling. The thermal settings can be past thermal settings applied to one of the past sleep sessions.

Some embodiments described herein include one or more methods that can be configured to perform one or more of the abovementioned systems and/or features.

The devices, system, and techniques described herein may provide one or more of the following advantages. For example, the disclosed technology provides non-invasive techniques for optimizing sleep quality of a user of a bed system. The disclosed technology leverages existing components of the bed system, such as temperature sensors that are attached to a top surface of a mattress of the bed system, to determine microclimate conditions during a past sleep session in which the user experienced a threshold level of sleep quality. The disclosed technology also leverages machine learning techniques and/or thermodynamic equations to model microclimate temperature data corresponding to the past sleep session with one or more thermal settings. The thermal settings can be designed/generated to copy or otherwise replicate the microclimate conditions during the past sleep session that caused the user to experience the threshold level of sleep quality. By executing the thermal settings during subsequent sleep sessions, the user can improve or otherwise maintain improved sleep quality. Accordingly, these improvements to sleep quality can result from non-invasively measuring microclimate conditions and continuously modeling/processing sleep quality data of past sleep sessions.

Similarly, the disclosed technology can provide for iteratively improving or adjusting the determined thermal settings as data continues to be collected during sleep sessions of the user. Therefore, the user's sleep environment can be dynamically modified to control different microclimate conditions that may cause the user to experience improved levels of sleep quality.

Moreover, analysis and modeling of past sleep sessions can be performed with remote compute resources, thereby leveraging processing power as well as efficient and accurate determinations to be made. Significant quantities of data collected throughout past sleep sessions of a user of a bed system can be transmitted to a cloud-based system and processed remotely from the bed system to generate robust and accurate microclimate optimization determinations for the particular user. At least some processing, such as determining when and what thermal routines to activate during a current sleep session of the user, can be performed on the edge (e.g., by a controller of the bed system, by a mobile computing device of the user) to provide for quick and accurate real-time adjustments to the bed system to ensure the user maintains or improves their level of sleep quality during the current sleep session. Therefore, both remote and edge compute resources can be leveraged with the disclosed technology to provide accurate and efficient microclimate optimization at the bed system of the user.

As another example, the disclosed technology can leverage robust historic sleep and/or microclimate data about a specific user as well as a population of users to determine thermal settings that control microclimate conditions of the user's bed system and, consequently, improve the user's sleep quality. More accurate determinations for controlling the microclimate conditions can be made by using such an abundance of data collected over many past sleep sessions of the user and/or the population of users.

Similarly, the disclosed technology can leverage machine learning models, algorithms, and techniques to accurately model data from past sleep sessions with thermal settings of a bed system. The modeled thermal settings can then be automatically applied to the bed system during subsequent sleep sessions of the user to control the microclimate conditions of the bed system and, consequently, improve the user's sleep quality.

As another example, the disclosed technology provides for personalization of a sleep experience. Individual thermal profiles can be established for each user of each bed system, based on temperature data unique to that particular user and threshold sleep quality information/data also unique to that user. The individualized thermal profiles can be used during future sleep sessions of the particular user to improve the overall sleep quality of that user. Similarly, the disclosed technology is adaptive to changing data about the particular user. The thermal profiles can change based on age of the user. For example, an older user may have distinct CBT changes in comparison to a child. The disclosed technology makes it possible to automatically update parameters of the individualized thermal profiles to adapt to changes associated with the user, such as changes in their age.

Moreover, the disclosed technology enables “open loop” regulation of thermal settings of a bed system. After all, the disclosed technology essentially reproduces thermal settings that were recorded in previous sleep sessions where a threshold level of sleep quality had been attained. The disclosed technology does not require additional consumption of compute resources and processing power to constantly adjust a microclimate of the bed system based on real-time sleep monitoring. As a result, compute resources and processing power can be more efficiently used to make other sleep-based determinations in real-time, during the sleep session, while the microclimate of the bed system is also automatically being adjusted based on the reproduced thermal settings.

The details of one or more implementations are set forth in the accompanying drawings and the description below. Other features, aspects and potential advantages will be apparent from the accompanying description and figures.

### DESCRIPTION OF DRAWINGS

FIG. 1 shows an example air bed system.

FIG. 2 is a block diagram of an example of various components of an air bed system.

FIG. 3 shows an example environment including a bed in communication with devices located in and around a home.

FIGS. 4A and 4B are block diagrams of example data processing systems that can be associated with a bed.

FIGS. 5 and 6 are block diagrams of examples of motherboards that can be used in a data processing system associated with a bed.

FIG. 7 is a block diagram of an example of a daughterboard that can be used in a data processing system associated with a bed.

FIG. 8 is a block diagram of an example of a motherboard with no daughterboard that can be used in a data processing system associated with a bed.

FIG. 9A is a block diagram of an example of a sensory array that can be used in a data processing system associated with a bed.

FIG. 9B is a schematic top view of a bed having an example of a sensor strip with one or more sensors that can be used in a data processing system associated with the bed.

FIG. 9C is a schematic diagram of an example bed with force sensors located at the bottom of legs of the bed.

FIG. 10 is a block diagram of an example of a control array that can be used in a data processing system associated with a bed

FIG. 11 is a block diagram of an example of a computing device that can be used in a data processing system associated with a bed.

FIGS. 12-16 are block diagrams of example cloud services that can be used in a data processing system associated with a bed.

FIG. 17 is a block diagram of an example of using a data processing system that can be associated with a bed to automate peripherals around the bed.

FIG. 18 is a schematic diagram that shows an example of a computing device and a mobile computing device.

FIG. 19A is a conceptual diagram for determining and applying thermal settings to a bed system for controlling a microclimate of the bed system and improving user sleep quality.

FIG. 19B is a flowchart of a process for controlling the microclimate of the bed system as shown in FIG. 19A.

FIG. 20 is a conceptual diagram of a process for determining microclimate temperature data of a bed system when a user rests on the bed system.

FIGS. 21A-B is a conceptual diagram of a process for determining thermal settings to implement during subsequent sleep sessions of a user based on processing data that corresponds to past sleep sessions of the user.

FIG. 22 is a conceptual diagram of a process for applying the thermal settings determined in the process of FIGS. 21A-B to improve the user's level of sleep quality during subsequent sleep sessions.

FIG. 23 is a conceptual diagram of a process to model microclimate temperature data collected at a bed system to thermal settings.

FIG. 24 is a flowchart of a process to determine microclimate temperature data of a bed system when a user rests on the bed system.

FIG. 25 is a flowchart of a process to determine thermal settings to implement during subsequent sleep sessions of a user based on processing data that corresponds to past sleep sessions of the user.

FIG. 26 is a flowchart of another process to determine thermal settings to implement during subsequent sleep sessions of a user based on processing data that corresponds to past sleep sessions of the user.

FIG. 27 is a flowchart of a process to determine, during a subsequent sleep session, when and what thermal settings to activate at a bed system to improve a user's level of sleep quality.

Like reference symbols in the various drawings indicate like elements.

### DETAILED DESCRIPTION

This document generally describes systems and techniques for controlling a microclimate of a bed system to improve levels of sleep quality of users of the bed system. Data associated with past sleep sessions of the user, such as microclimate temperature data and sleep quality data, can be processed, by a computer system, to identify microclimate conditions during a past sleep session that resulted in the user experiences at least a threshold level of sleep quality. The computer system can then model the microclimate temperature data of the identified past sleep session to determine thermal settings to implement during subsequent sleep sessions that can replicate the microclimate conditions during the identified past sleep session. As a result of such techniques to control the microclimate conditions of the bed system during the subsequent sleep sessions, the user may improve and/or maintain their sleep quality.

Example Airbed Hardware

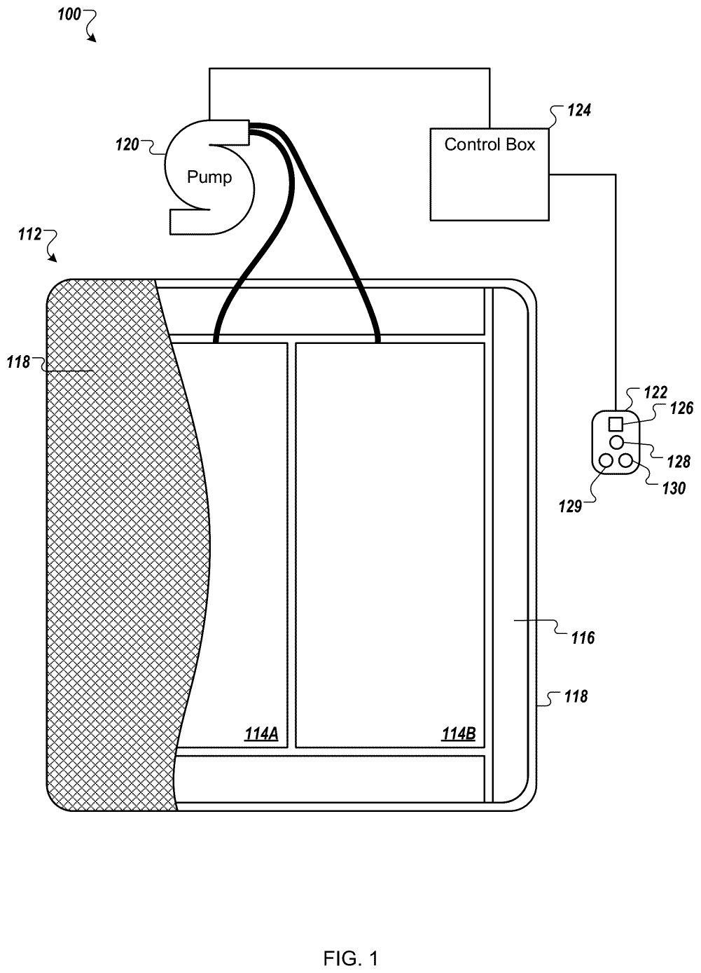

FIG. 1 shows an example air bed system 100 that includes a bed 112. The bed 112 can be a mattress that includes at least one air chamber 114 surrounded by a resilient border 116 and encapsulated by bed ticking 118. The resilient border 116 can comprise any suitable material, such as foam. In some embodiments, the resilient border 116 can combine with a top layer or layers of foam (not shown in FIG. 1) to form an upside down foam tub. In other embodiments, mattress structure can be varied as suitable for the application.

As illustrated in FIG. 1, the bed 112 can be a two chamber design having first and second fluid chambers, such as a first air chamber 114A and a second air chamber 114B. Sometimes, the bed 112 can include chambers for use with fluids other than air that are suitable for the application. For example, the fluids can include liquid. In some embodiments, such as single beds or kids' beds, the bed 112 can include a single air chamber 114A or 114B or multiple air chambers 114A and 114B. Although not depicted, sometimes, the bed 112 can include additional air chambers.

The first and second air chambers 114A and 114B can be in fluid communication with a pump 120. The pump 120 can be in electrical communication with a remote control 122 via control box 124. The control box 124 can include a wired or wireless communications interface for communicating with one or more devices, including the remote control 122. The control box 124 can be configured to operate the pump 120 to cause increases and decreases in the fluid pressure of the first and second air chambers 114A and 114B based upon commands input by a user using the remote control 122. In some implementations, the control box 124 is integrated into a housing of the pump 120. Moreover, sometimes, the pump 120 can be in wireless communication (e.g., via a home network, WIFI, BLUETOOTH, or other wireless network) with a mobile device via the control box 124. The mobile device can include but is not limited to the user's smartphone, cell phone, laptop, tablet, computer, wearable device, home automation device, or other computing device. A mobile application can be presented at the mobile device and provide functionality for the user to control the bed 112 and view information about the bed 112. The user can input commands in the mobile application presented at the mobile device. The inputted commands can be transmitted to the control box 124, which can operate the pump 120 based upon the commands.

The remote control 122 can include a display 126, an output selecting mechanism 128, a pressure increase button 129, and a pressure decrease button 130. The remote control 122 can include one or more additional output selecting mechanisms and/or buttons. The display 126 can present information to the user about settings of the bed 112. For example, the display 126 can present pressure settings of both the first and second air chambers 114A and 114B or one of the first and second air chambers 114A and 114B. Sometimes, the display 126 can be a touch screen, and can receive input from the user indicating one or more commands to control pressure in the first and second air chambers 114A and 114B and/or other settings of the bed 112.

The output selecting mechanism 128 can allow the user to switch air flow generated by the pump 120 between the first and second air chambers 114A and 114B, thus enabling control of multiple air chambers with a single remote control 122 and a single pump 120. For example, the output selecting mechanism 128 can by a physical control (e.g., switch or button) or an input control presented on the display 126. Alternatively, separate remote control units can be provided for each air chamber 114A and 114B and can each include the ability to control multiple air chambers. Pressure increase and decrease buttons 129 and 130 can allow the user to increase or decrease the pressure, respectively, in the air chamber selected with the output selecting mechanism 128. Adjusting the pressure within the selected air chamber can cause a corresponding adjustment to the firmness of the respective air chamber. In some embodiments, the remote control 122 can be omitted or modified as appropriate for an application.

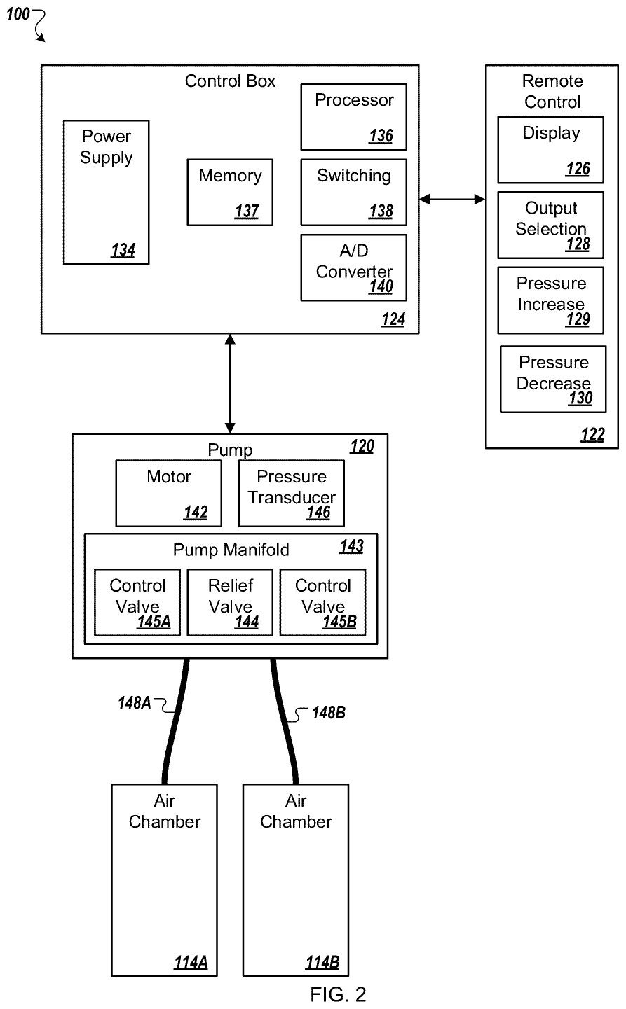

FIG. 2 is a block diagram of an example of various components of an air bed system. These components can be used in the example air bed system 100. The control box 124 can include a power supply 134, a processor 136, a memory 137, a switching mechanism 138, and an analog to digital (A/D) converter 140. The switching mechanism 138 can be, for example, a relay or a solid state switch. In some implementations, the switching mechanism 138 can be located in the pump 120 rather than the control box 124. The pump 120 and the remote control 122 can be in two-way communication with the control box 124. The pump 120 includes a motor 142, a pump manifold 143, a relief valve 144, a first control valve 145A, a second control valve 145B, and a pressure transducer 146. The pump 120 is fluidly connected with the first air chamber 114A and the second air chamber 114B via a first tube 148A and a second tube 148B, respectively. The first and second control valves 145A and 145B can be controlled by switching mechanism 138, and are operable to regulate the flow of fluid between the pump 120 and first and second air chambers 114A and 114B, respectively.

In some implementations, the pump 120 and the control box 124 can be provided and packaged as a single unit. In some implementations, the pump 120 and the control box 124 can be provided as physically separate units. The control box 124, the pump 120, or both can be integrated within or otherwise contained within a bed frame, foundation, or bed support structure that supports the bed 112. Sometimes, the control box 124, the pump 120, or both can be located outside of a bed frame, foundation, or bed support structure (as shown in the example in FIG. 1).

The air bed system 100 in FIG. 2 includes the two air chambers 114A and 114B and the single pump 120 of the bed 112 depicted in FIG. 1. However, other implementations can include an air bed system having two or more air chambers and one or more pumps incorporated into the air bed system to control the air chambers. For example, a separate pump can be associated with each air chamber. As another example, a pump can be associated with multiple chambers. A first pump can be associated with air chambers that extend longitudinally from a left side to a midpoint of the air bed system 100 and a second pump can be associated with air chambers that extend longitudinally from a right side to the midpoint of the air bed system 100. Separate pumps can allow each air chamber to be inflated or deflated independently and/or simultaneously. Additional pressure transducers can also be incorporated into the air bed system 100 such that a separate pressure transducer can be associated with each air chamber.

As an illustrative example, in use, the processor 136 can send a decrease pressure command to one of air chambers 114A or 114B, and the switching mechanism 138 can convert the low voltage command signals sent by the processor 136 to higher operating voltages sufficient to operate the relief valve 144 of the pump 120 and open the respective control valve 145A or 145B. Opening the relief valve 144 can allow air to escape from the air chamber 114A or 114B through the respective air tube 148A or 148B. During deflation, the pressure transducer 146 can send pressure readings to the processor 136 via the A/D converter 140. The A/D converter 140 can receive analog information from pressure transducer 146 and can convert the analog information to digital information useable by the processor 136. The processor 136 can send the digital signal to the remote control 122 to update the display 126 to convey the pressure information to the user. The processor 136 can also send the digital signal to other devices in wired or wireless communication with the air bed system, including but not limited to mobile devices described herein. The user can then view pressure information associated with the air bed system at their device instead of at, or in addition to, the remote control 122.

As another example, the processor 136 can send an increase pressure command. The pump motor 142 can be energized in response to the increase pressure command and send air to the designated one of the air chambers 114A or 114B through the air tube 148A or 148B via electronically operating the corresponding valve 145A or 145B. While air is being delivered to the designated air chamber 114A or 114B to increase the chamber firmness, the pressure transducer 146 can sense pressure within the pump manifold 143. The pressure transducer 146 can send pressure readings to the processor 136 via the A/D converter 140. The processor 136 can use the information received from the A/D converter 140 to determine the difference between the actual pressure in air chamber 114A or 114B and the desired pressure. The processor 136 can send the digital signal to the remote control 122 to update display 126.

Generally speaking, during an inflation or deflation process, the pressure sensed within the pump manifold 143 can provide an approximation of the actual pressure within the respective air chamber that is in fluid communication with the pump manifold 143. An example method includes turning off the pump 120, allowing the pressure within the air chamber 114A or 114B and the pump manifold 143 to equalize, then sensing the pressure within the pump manifold 143 with the pressure transducer 146. Providing a sufficient amount of time to allow the pressures within the pump manifold 143 and chamber 114A or 114B to equalize can result in pressure readings that are accurate approximations of actual pressure within air chamber 114A or 114B. In some implementations, the pressure of the air chambers 114A and/or 114B can be continuously monitored using multiple pressure sensors (not shown). The pressure sensors can be positioned within the air chambers. The pressure sensors can also be fluidly connected to the air chambers, such as along the air tubes 148A and 148B.

In some implementations, information collected by the pressure transducer 146 can be analyzed to determine various states of a user laying on the bed 112. For example, the processor 136 can use information collected by the pressure transducer 146 to determine a heartrate or a respiration rate for the user. As an illustrative example, the user can be laying on a side of the bed 112 that includes the chamber 114A. The pressure transducer 146 can monitor fluctuations in pressure of the chamber 114A, and this information can be used to determine the user's heartrate and/or respiration rate. As another example, additional processing can be performed using the collected data to determine a sleep state of the user (e.g., awake, light sleep, deep sleep). For example, the processor 136 can determine when the user falls asleep and, while asleep, the various sleep states (e.g., sleep stages) of the user. Based on the determined heartrate, respiration rate, and/or sleep states of the user, the processor 136 can determine information about the user's sleep quality. The processor 136 can, for example, determine how well the user slept during a particular sleep cycle. The processor 136 can also determine user sleep cycle trends. Accordingly, the processor 136 can generate recommendations to improve the user's sleep quality and overall sleep cycle. Information that is determined about the user's sleep cycle (e.g., heartrate, respiration rate, sleep states, sleep quality, recommendations to improve sleep quality, etc.) can be transmitted to the user's mobile device and presented in a mobile application, as described above.

Additional information associated with the user of the air bed system 100 that can be determined using information collected by the pressure transducer 146 includes user motion, presence on a surface of the bed 112, weight, heart arrhythmia, snoring, partner snore, and apnea. One or more other health conditions of the user can also be determined based on the information collected by the pressure transducer 146. Taking user presence detection for example, the pressure transducer 146 can be used to detect the user's presence on the bed 112, e.g., via a gross pressure change determination and/or via one or more of a respiration rate signal, heartrate signal, and/or other biometric signals. Detection of the user's presence can be beneficial to determine, by the processor 136, adjustment(s) to make to settings of the bed 112 (e.g., adjusting a firmness when the user is present to a user-preferred firmness setting) and/or peripheral devices (e.g., turning off lights when the user is present, activating a heating or cooling system, etc.).

For example, a simple pressure detection process can identify an increase in pressure as an indication that the user is present. As another example, the processor 136 can determine that the user is present if the detected pressure increases above a specified threshold (so as to indicate that a person or other object above a certain weight is positioned on the bed 112). As yet another example, the processor 136 can identify an increase in pressure in combination with detected slight, rhythmic fluctuations in pressure as corresponding to the user being present. The presence of rhythmic fluctuations can be identified as being caused by respiration or heart rhythm (or both) of the user. The detection of respiration or a heartbeat can distinguish between the user being present on the bed and another object (e.g., a suitcase, a pet, a pillow, etc.) being placed thereon.

In some implementations, pressure fluctuations can be measured at the pump 120. For example, one or more pressure sensors can be located within one or more internal cavities of the pump 120 to detect pressure fluctuations within the pump 120. The fluctuations detected at the pump 120 can indicate pressure fluctuations in the chambers 114A and/or 114B. One or more sensors located at the pump 120 can be in fluid communication with the chambers 114A and/or 114B, and the sensors can be operative to determine pressure within the chambers 114A and/or 114B. The control box 124 can be configured to determine at least one vital sign (e.g., heartrate, respiratory rate) based on the pressure within the chamber 114A or the chamber 114B.

The control box 124 can also analyze a pressure signal detected by one or more pressure sensors to determine a heartrate, respiration rate, and/or other vital signs of the user lying or sitting on the chamber 114A and/or 114B. More specifically, when a user lies on the bed 112 and is positioned over the chamber 114A, each of the user's heart beats, breaths, and other movements (e.g., hand, arm, leg, foot, or other gross body movements) can create a force on the bed 112 that is transmitted to the chamber 114A. As a result of this force input, a wave can propagate through the chamber 114A and into the pump 120. A pressure sensor located at the pump 120 can detect the wave, and thus the pressure signal outputted by the sensor can indicate a heartrate, respiratory rate, or other information regarding the user.

With regard to sleep state, the air bed system 100 can determine the user's sleep state by using various biometric signals such as heartrate, respiration, and/or movement of the user. While the user is sleeping, the processor 136 can receive one or more of the user's biometric signals (e.g., heartrate, respiration, motion, etc.) and can determine the user's present sleep state based on the received biometric signals. In some implementations, signals indicating fluctuations in pressure in one or both of the chambers 114A and 114B can be amplified and/or filtered to allow for more precise detection of heartrate and respiratory rate.

Sometimes, the processor 136 can receive additional biometric signals of the user from one or more other sensors or sensor arrays positioned on or otherwise integrated into the air bed system 100. For example, one or more sensors can be attached or removably attached to a top surface of the air bed system 100 and configured to detect signals such as heartrate, respiration rate, and/or motion. The processor 136 can combine biometric signals received from pressure sensors located at the pump 120, the pressure transducer 146, and/or the sensors positioned throughout the air bed system 100 to generate accurate and more precise information about the user and their sleep quality.

Sometimes, the control box 124 can perform a pattern recognition algorithm or other calculation based on the amplified and filtered pressure signal(s) to determine the user's heartrate and/or respiratory rate. For example, the algorithm or calculation can be based on assumptions that a heartrate portion of the signal has a frequency in a range of 0.5-4.0 Hz and that a respiration rate portion of the signal has a frequency in a range of less than 1 Hz. Sometimes, the control box 124 can use one or more machine learning models to determine the user's health information. The models can be trained using training data that includes training pressure signals and expected heartrates and/or respiratory rates. Sometimes, the control box 124 can determine user health information by using a lookup table that corresponds to sensed pressure signals.

The control box 124 can also be configured to determine other characteristics of the user based on the received pressure signal, such as blood pressure, tossing and turning movements, rolling movements, limb movements, weight, presence or lack of presence of the user, and/or the identity of the user.

For example, the pressure transducer 146 can be used to monitor the air pressure in the chambers 114A and 114B of the bed 112. If the user on the bed 112 is not moving, the air pressure changes in the air chamber 114A or 114B can be relatively minimal, and can be attributable to respiration and/or heartbeat. When the user on the bed 112 is moving, however, the air pressure in the mattress can fluctuate by a much larger amount. The pressure signals generated by the pressure transducer 146 and received by the processor 136 can be filtered and indicated as corresponding to motion, heartbeat, or respiration. The processor 136 can attribute such fluctuations in air pressure to the user's sleep quality. Such attributions can be determined based on applying one or more machine learning models and/or algorithms to the pressure signals. For example, if the user shifts and turns a lot during a sleep cycle (for example, in comparison to historic trends of the user's sleep cycles), the processor 136 can determine that the user experienced poor sleep during that particular sleep cycle.

In some implementations, rather than performing the data analysis in the control box 124 with the processor 136, a digital signal processor (DSP) can be provided to analyze the data collected by the pressure transducer 146. Alternatively, the collected data can be sent to a cloud-based computing system for remote analysis.

In some implementations, the example air bed system 100 further includes a temperature controller configured to increase, decrease, or maintain a temperature of the bed 112, for example for the comfort of the user. For example, a pad (e.g., mat, layer, etc.) can be placed on top of or be part of the bed 112, or can be placed on top of or be part of one or both of the chambers 114A and 114B. Air can be pushed through the pad and vented to cool off the user on the bed 112. Additionally or alternatively, the pad can include a heating element used to keep the user warm. In some implementations, the temperature controller can receive temperature readings from the pad. The temperature controller can determine whether the temperature readings are less than or greater than some threshold range and/or value. Based on this determination, the temperature controller can actuate components to push air through the pad to cool off the user or active the heating element. In some implementations, separate pads are used for different sides of the bed 112 (e.g., corresponding to the locations of the chambers 114A and 114B) to provide for differing temperature control for the different sides of the bed 112. Each pad can be selectively controlled by the temperature controller to provide cooling or heating preferred by each user on the different sides of the bed 112. For example, a first user on a left side of the bed 112 can prefer to have their side of the bed 112 cooled during the night while a second user on a right side of the bed 112 can prefer to have their side of the bed 112 warmed during the night.

In some implementations, the user of the air bed system 100 can use an input device, such as the remote control 122 or a mobile device as described above, to input a desired temperature for a surface of the bed 112 (or for a portion of the surface of the bed 112, for example at a foot region, a lumbar or waist region, a shoulder region, and/or a head region of the bed 112). The desired temperature can be encapsulated in a command data structure that includes the desired temperature and also identifies the temperature controller as the desired component to be controlled. The command data structure can then be transmitted via Bluetooth or another suitable communication protocol (e.g., WIFI, a local network, etc.) to the processor 136. In various examples, the command data structure is encrypted before being transmitted. The temperature controller can then configure its elements to increase or decrease the temperature of the pad depending on the temperature input provided at the remote control 122 by the user.

In some implementations, data can be transmitted from a component back to the processor 136 or to one or more display devices, such as the display 126 of the remote controller 122. For example, the current temperature as determined by a sensor element of a temperature controller, the pressure of the bed, the current position of the foundation or other information can be transmitted to control box 124. The control box 124 can transmit this information to the remote control 122 to be displayed to the user (e.g., on the display 126). As described above, the control box 124 can also transmit the received information to a mobile device to be displayed in a mobile application or other graphical user interface (GUI) to the user.

In some implementations, the example air bed system 100 further includes an adjustable foundation and an articulation controller configured to adjust the position of the bed 112 by adjusting the adjustable foundation supporting the bed. For example, the articulation controller can adjust the bed 112 from a flat position to a position in which a head portion of a mattress of the bed is inclined upward (e.g., to facilitate a user sitting up in bed and/or watching television). The bed 112 can also include multiple separately articulable sections. As an illustrative example, the bed 112 can include one or more of a head portion, a lumbar/waist portion, a leg portion, and/or a foot portion, all of which can be separately articulable. As another example, portions of the bed 112 corresponding to the locations of the chambers 114A and 114B can be articulated independently from each other, to allow one user positioned on the bed 112 surface to rest in a first position (e.g., a flat position or other desired position) while a second user rests in a second position (e.g., a reclining position with the head raised at an angle from the waist or another desired position). Separate positions can also be set for two different beds (e.g., two twin beds placed next to each other). The foundation of the bed 112 can include more than one zone that can be independently adjusted.

Sometimes, the bed 112 can be adjusted to one or more user-defined positions based on user input and/or user preferences. For example, the bed 112 can automatically adjust, by the articulation controller, to one or more user-defined settings. As another example, the user can control the articulation controller to adjust the bed 112 to one or more user-defined positions. Sometimes, the bed 112 can be adjusted to one or more positions that may provide the user with improved or otherwise improve sleep and sleep quality. For example, a head portion on one side of the bed 112 can be automatically articulated, by the articulation controller, when one or more sensors of the air bed system 100 detect that a user sleeping on that side of the bed 112 is snoring. As a result, the user's snoring can be mitigated so that the snoring does not wake up another user sleeping in the bed 112.

In some implementations, the bed 112 can be adjusted using one or more devices in communication with the articulation controller or instead of the articulation controller. For example, the user can change positions of one or more portions of the bed 112 using the remote control 122 described above. The user can also adjust the bed 112 using a mobile application or other graphical user interface presented at a mobile computing device of the user.

The articulation controller can also provide different levels of massage to one or more portions of the bed 112 for one or more users. The user(s) can adjust one or more massage settings for the portions of the bed 112 using the remote control 122 and/or a mobile device in communication with the air bed system 100.

Example of a Bed in a Bedroom Environment

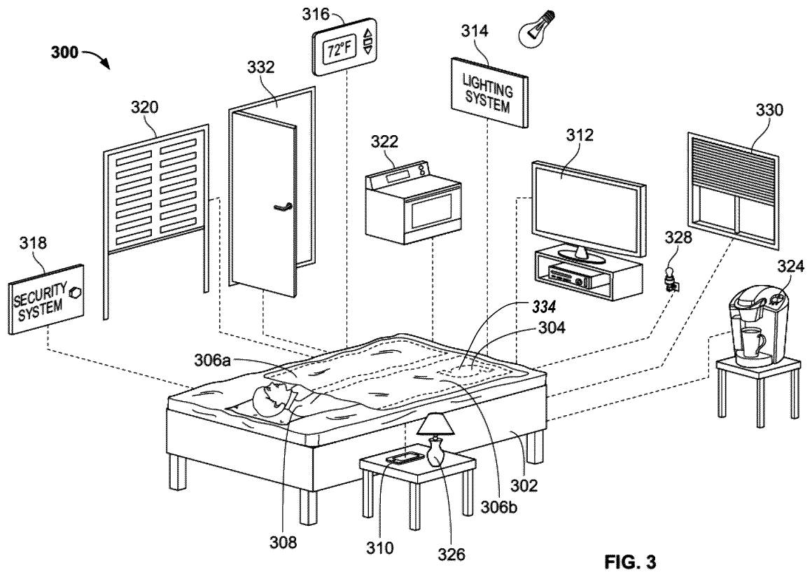

FIG. 3 shows an example environment 300 including a bed 302 in communication with devices located in and around a home. In the example shown, the bed 302 includes pump 304 for controlling air pressure within two air chambers 306a and 306b (as described above). The pump 304 additionally includes circuitry 334 for controlling inflation and deflation functionality performed by the pump 304. The circuitry 334 is programmed to detect fluctuations in air pressure of the air chambers 306a-b and use the detected fluctuations to identify bed presence of a user 308, the user's sleep state, movement, and biometric signals (e.g., heartrate, respiration rate). The detected fluctuations can also be used to detect when the user 308 is snoring and whether the user 308 has sleep apnea or other health conditions. The detected fluctuations can also be used to determine an overall sleep quality of the user 308.

In the example shown, the pump 304 is located within a support structure of the bed 302 and the control circuitry 334 for controlling the pump 304 is integrated with the pump 304. In some implementations, the control circuitry 334 is physically separate from the pump 304 and is in wireless or wired communication with the pump 304. In some implementations, the pump 304 and/or control circuitry 334 are located outside of the bed 302. In some implementations, various control functions can be performed by systems located in different physical locations. For example, circuitry for controlling actions of the pump 304 can be located within a pump casing of the pump 304 while control circuitry 334 for performing other functions associated with the bed 302 can be located in another portion of the bed 302, or external to the bed 302. The control circuitry 334 located within the pump 304 can also communicate with control circuitry 334 at a remote location through a LAN or WAN (e.g., the internet). The control circuitry 334 can also be included in the control box 124 of FIGS. 1 and 2.

In some implementations, one or more devices other than, or in addition to, the pump 304 and control circuitry 334 can be utilized to identify user bed presence, sleep state, movement, biometric signals, and other information (e.g., sleep quality, health related) about the user 308. For example, the bed 302 can include a second pump, with each pump connected to a respective one of the air chambers 306a-b. For example, the pump 304 can be in fluid communication with the air chamber 306b to control inflation and deflation of the air chamber 306b as well as detect user signals for a user located over the air chamber 306b. The second pump can be in fluid communication with the air chamber 306a and used to control inflation and deflation of the air chamber 306a as well as detect user signals for a user located over the air chamber 306a.

As another example, the bed 302 can include one or more pressure sensitive pads or surface portions operable to detect movement, including user presence, motion, respiration, and heartrate. A first pressure sensitive pad can be incorporated into a surface of the bed 302 over a left portion of the bed 302, where a first user would normally be located during sleep, and a second pressure sensitive pad can be incorporated into the surface of the bed 302 over a right portion of the bed 302, where a second user would normally be located. The movement detected by the pressure sensitive pad(s) or surface portion(s) can be used by control circuitry 334 to identify user sleep state, bed presence, or biometric signals for each user. The pressure sensitive pads can also be removable rather than incorporated into the surface of the bed 302.

The bed 302 can also include one or more temperature sensors and/or array of sensors operable to detect temperatures in microclimates of the bed 302. Detected temperatures in different microclimates of the bed 302 can be used by the control circuitry 334 to determine one or more modifications to the user 308's sleep environment. For example, a temperature sensor located near a core region of the bed 302 where the user 308 rests can detect high temperature values. Such high temperature values can indicate that the user 308 is warm. To lower the user's body temperature in this microclimate, the control circuitry 334 can determine that a cooling element of the bed 302 can be activated. As another example, the control circuitry 334 can determine that a cooling unit in the home can be automatically activated to cool an ambient temperature in the environment 300.

The control circuitry 334 can also process a combination of signals sensed by different sensors that are integrated into, positioned on, or otherwise in communication with the bed 112. For example, pressure and temperature signals can be processed by the control circuitry 334 to more accurately determine one or more health conditions of the user 308 and/or sleep quality of the user 308. Acoustic signals detected by one or more microphones or other audio sensors can also be used in combination with pressure or motion sensors in order to determine when the user 308 snores, whether the user 308 has sleep apnea, and/or overall sleep quality of the user 308. Combinations of one or more other sensed signals are also possible for the control circuitry 334 to more accurately determine one or more health and/or sleep conditions of the user 308.

Accordingly, information detected by one or more sensors or other components of the bed 112 (e.g., motion information) can be processed by the control circuitry 334 and provided to one or more user devices, such as a user device 310 for presentation to the user 308 or to other users. The information can be presented in a mobile application or other graphical user interface at the user device 310. The user 308 can view different information that is processed and/or determined by the control circuitry 334 and based the signals that are detected by components of the bed 302. For example, the user 308 can view their overall sleep quality for a particular sleep cycle (e.g., the previous night), historic trends of their sleep quality, and health information. The user 308 can also adjust one or more settings of the bed 302 (e.g., increase or decrease pressure in one or more regions of the bed 302, incline or decline different regions of the bed 302, turn on or off massage features of the bed 302, etc.) using the mobile application that is presented at the user device 310.

In the example depicted in FIG. 3, the user device 310 is a mobile phone; however, the user device 310 can also be any one of a tablet, personal computer, laptop, a smartphone, a smart television (e.g., a television 312), a home automation device, or other user device capable of wired or wireless communication with the control circuitry 334, one or more other components of the bed 302, and/or one or more devices in the environment 300. The user device 310 can be in communication with the control circuitry 334 of the bed 302 through a network or through direct point-to-point communication. For example, the control circuitry 334 can be connected to a LAN (e.g., through a WIFI router) and communicate with the user device 310 through the LAN. As another example, the control circuitry 334 and the user device 310 can both connect to the Internet and communicate through the Internet. For example, the control circuitry 334 can connect to the Internet through a WIFI router and the user device 310 can connect to the Internet through communication with a cellular communication system. As another example, the control circuitry 334 can communicate directly with the user device 310 through a wireless communication protocol, such as Bluetooth. As yet another example, the control circuitry 334 can communicate with the user device 310 through a wireless communication protocol, such as ZigBee, Z-Wave, infrared, or another wireless communication protocol suitable for the application. As another example, the control circuitry 334 can communicate with the user device 310 through a wired connection such as, for example, a USB connector, serial/RS232, or another wired connection suitable for the application.

As mentioned above, the user device 310 can display a variety of information and statistics related to sleep, or user 308's interaction with the bed 302. For example, a user interface displayed by the user device 310 can present information including amount of sleep for the user 308 over a period of time (e.g., a single evening, a week, a month, etc.), amount of deep sleep, ratio of deep sleep to restless sleep, time lapse between the user 308 getting into bed and falling asleep, total amount of time spent in the bed 302 for a given period of time, heartrate over a period of time, respiration rate over a period of time, or other information related to user interaction with the bed 302 by the user 308 or one or more other users. In some implementations, information for multiple users can be presented on the user device 310, for example information for a first user positioned over the air chamber 306a can be presented along with information for a second user positioned over the air chamber 306b. In some implementations, the information presented on the user device 310 can vary according to the age of the user 308 so that the information presented evolves with the age of the user 308.

The user device 310 can also be used as an interface for the control circuitry 334 of the bed 302 to allow the user 308 to enter information and/or adjust one or more settings of the bed 302. The information entered by the user 308 can be used by the control circuitry 334 to provide better information to the user 308 or to various control signals for controlling functions of the bed 302 or other devices. For example, the user 308 can enter information such as weight, height, and age of the user 308. The control circuitry 334 can use this information to provide the user 308 with a comparison of the user 308's tracked sleep information to sleep information of other people having similar weights, heights, and/or ages as the user 308. The control circuitry 308 can also use this information to accurately determine overall sleep quality and/or health of the user 308 based on information detected by components (e.g., sensors) of the bed 302.

The user 308 may also use the user device 310 as an interface for controlling air pressure of the air chambers 306a and 306b, various recline or incline positions of the bed 302, temperature of one or more surface temperature control devices of the bed 302, or for allowing the control circuitry 334 to generate control signals for other devices (as described below).

The control circuitry 334 may also communicate with other devices or systems, including but not limited to the television 312, a lighting system 314, a thermostat 316, a security system 318, home automation devices, and/or other household devices (e.g., an oven 322, a coffee maker 324, a lamp 326, a nightlight 328). Other examples of devices and/or systems include a system for controlling window blinds 330, devices for detecting or controlling states of one or more doors 332 (such as detecting if a door is open, detecting if a door is locked, or automatically locking a door), and a system for controlling a garage door 320 (e.g., control circuitry 334 integrated with a garage door opener for identifying an open or closed state of the garage door 320 and for causing the garage door opener to open or close the garage door 320). Communications between the control circuitry 334 and other devices can occur through a network (e.g., a LAN or the Internet) or as point-to-point communication (e.g., BLUETOOTH, radio communication, or a wired connection). Control circuitry 334 of different beds 302 can also communicate with different sets of devices. For example, a kid's bed may not communicate with and/or control the same devices as an adult bed. In some embodiments, the bed 302 can evolve with the age of the user such that the control circuitry 334 of the bed 302 communicates with different devices as a function of age of the user of that bed 302.

The control circuitry 334 can receive information and inputs from other devices/systems and use the received information and inputs to control actions of the bed 302 and/or other devices. For example, the control circuitry 334 can receive information from the thermostat 316 indicating a current environmental temperature for a house or room in which the bed 302 is located. The control circuitry 334 can use the received information (along with other information, such as signals detected from one or more sensors of the bed 302) to determine if a temperature of all or a portion of the surface of the bed 302 should be raised or lowered. The control circuitry 334 can then cause a heating or cooling mechanism of the bed 302 to raise or lower the temperature of the surface of the bed 302. The control circuitry 334 can also cause a heating or cooling unit of the house or room in which the bed 302 is located to raise or lower the ambient temperature surrounding the bed 302. Thus, by adjusting the temperature of the bed 302 and/or the room in which the bed 302 is located, the user 308 can experience more improved sleep quality and comfort.

As an example, the user 308 can indicate a desired sleeping temperature of 74 degrees while a second user of the bed 302 indicates a desired sleeping temperature of 72 degrees. The thermostat 316 can transmit signals indicating room temperature at predetermined times to the control circuitry 334. The thermostat 316 can also send a continuous stream of detected temperature values of the room to the control circuitry 334. The transmitted signal(s) can indicate to the control circuitry 334 that the current temperature of the bedroom is 72 degrees. The control circuitry 334 can identify that the user 308 has indicated a desired sleeping temperature of 74 degrees, and can accordingly send control signals to a heating pad located on the user 308's side of the bed to raise the temperature of the portion of the surface of the bed 302 where the user 308 is located until the user 308's desired temperature is achieved. Moreover, the control circuitry 334 can sent control signals to the thermostat 316 and/or a heating unit in the house to raise the temperature in the room in which the bed 302 is located.

The control circuitry 334 can generate control signals to control other devices and propagate the control signals to the other devices. The control signals can be generated based on information collected by the control circuitry 334, including information related to user interaction with the bed 302 by the user 308 and/or one or more other users. Information collected from other devices other than the bed 302 can also be used when generating the control signals. For example, information relating to environmental occurrences (e.g., environmental temperature, environmental noise level, and environmental light level), time of day, time of year, day of the week, or other information can be used when generating control signals for various devices in communication with the control circuitry 334 of the bed 302.

For example, information on the time of day can be combined with information relating to movement and bed presence of the user 308 to generate control signals for the lighting system 314. The control circuitry 334 can, based on detected pressure signals of the user 308 on the bed 302, determine when the user 308 is presently in the bed 302 and when the user 308 falls asleep. Once the control circuitry 334 determines that the user has fallen asleep, the control circuitry 334 can transmit control signals to the lighting system 314 to turn off lights in the room in which the bed 302 is located, to lower the window blinds 330 in the room, and/or to activate the nightlight 328. Moreover, the control circuitry 334 can receive input from the user 308 (e.g., via the user device 310) that indicates a time at which the user 308 would like to wake up. When that time approaches, the control circuitry 334 can transmit control signals to one or more devices in the environment 300 to control devices that may cause the user 308 to wake up. For example, the control signals can be sent to a home automation device that controls multiple devices in the home. The home automation device can be instructed, by the control circuitry 334, to raise the window blinds 330, turn off the nightlight 328, turn on lighting beneath the bed 302, start the coffee machine 324, change a temperature in the house via the thermostat 316, or perform some other home automation. The home automation device can also be instructed to activate an alarm that can cause the user 308 to wake up. Sometimes, the user 308 can input information at the user device 310 that indicates what actions can be taken by the home automation device or other devices in the environment 300.

In some implementations, rather than or in addition to providing control signals for other devices, the control circuitry 334 can provide collected information (e.g., information related to user movement, bed presence, sleep state, or biometric signals) to one or more other devices to allow the one or more other devices to utilize the collected information when generating control signals. For example, the control circuitry 334 of the bed 302 can provide information relating to user interactions with the bed 302 by the user 308 to a central controller (not shown) that can use the provided information to generate control signals for various devices, including the bed 302.

The central controller can, for example, be a hub device that provides a variety of information about the user 308 and control information associated with the bed 302 and other devices in the house. The central controller can include sensors that detect signals that can be used by the control circuitry 334 and/or the central controller to determine information about the user 308 (e.g., biometric or other health data, sleep quality). The sensors can detect signals including such as ambient light, temperature, humidity, volatile organic compound(s), pulse, motion, and audio. These signals can be combined with signals detected by sensors of the bed 302 to determine accurate information about the user 308's health and sleep quality. The central controller can provide controls (e.g., user-defined, presets, automated, user initiated) for the bed 302, determining and viewing sleep quality and health information, a smart alarm clock, a speaker or other home automation device, a smart picture frame, a nightlight, and one or more mobile applications that the user 308 can install and use at the central controller. The central controller can include a display screen that outputs information and receives user input. The display can output information such as the user 308's health, sleep quality, weather, security integration features, lighting integration features, heating and cooling integration features, and other controls to automate devices in the house. The central controller can operate to provide the user 308 with functionality and control of multiple different types of devices in the house as well as the user 308's bed 302.

As an illustrative example of FIG. 3, the control circuitry 334 integrated with the pump 304 can detect a feature of a mattress of the bed 302, such as an increase in pressure in the air chamber 306b, and use this detected increase to determine that the user 308 is present on the bed 302. The control circuitry 334 may also identify a heartrate or respiratory rate for the user 308 to identify that the increased pressure is due to a person sitting, laying, or resting on the bed 302, rather than an inanimate object (e.g., a suitcase) having been placed on the bed 302. In some implementations, the information indicating user bed presence can be combined with other information to identify a current or future likely state for the user 308. For example, a detected user bed presence at 11:00 am can indicate that the user is sitting on the bed (e.g., to tie her shoes, or to read a book) and does not intend to go to sleep, while a detected user bed presence at 10:00 pm can indicate that the user 308 is in bed for the evening and is intending to fall asleep soon. As another example, if the control circuitry 334 detects that the user 308 has left the bed 302 at 6:30 am (e.g., indicating that the user 308 has woken up for the day), and then later detects presence of the user 308 at 7:30 am on the bed 302, the control circuitry 334 can use this information that the newly detected presence is likely temporary (e.g., while the user 308 ties her shoes before heading to work) rather than an indication that the user 308 is intending to stay on the bed 302 for an extended period of time.

If the control circuitry 334 determines that the user 308 is likely to remain on the bed 302 for an extended period of time, the control circuitry 334 can determine one or more home automation controls that can aid the user 308 in falling asleep and experience improved sleep quality throughout the user 308's sleep cycle. For example, the control circuitry 334 can communicate with security system 318 to ensure that doors are locked. The control circuitry 334 can communicate with the oven 322 to ensure that the oven 322 is turned off. The control circuitry 334 can also communicate with the lighting system 314 to dim or otherwise turn off lights in the room in which the bed 302 is located and/or throughout the house, and the control circuitry 334 can communicate with the thermostat 316 to ensure that the house is at a desired temperature of the user 308. The control circuitry 334 can also determine one or more adjustments that can be made to the bed 302 to facilitate the user 308 falling asleep and staying asleep (e.g., changing a position of one or more regions of the bed 302, foot warming, massage features, pressure/firmness in one or more regions of the bed 302, etc.).

In some implementations, the control circuitry 334 may use collected information (including information related to user interaction with the bed 302 by the user 308, environmental information, time information, and user input) to identify use patterns for the user 308. For example, the control circuitry 334 can use information indicating bed presence and sleep states for the user 308 collected over a period of time to identify a sleep pattern for the user. The control circuitry 334 can identify that the user 308 generally goes to bed between 9:30 pm and 10:00 pm, generally falls asleep between 10:00 pm and 11:00 pm, and generally wakes up between 6:30 am and 6:45 am, based on information indicating user presence and biometrics for the user 308 collected over a week or a different time period. The control circuitry 334 can use identified patterns of the user 308 to better process and identify user interactions with the bed 302.

Given the above example user bed presence, sleep, and wake patterns for the user 308, if the user 308 is detected as being on the bed 302 at 3:00 pm, the control circuitry 334 can determine that the user 308's presence on the bed 302 is temporary, and use this determination to generate different control signals than if the control circuitry 334 determined the user 308 was in bed for the evening (e.g., at 3:00 pm, a head region of the bed 302 can be raised to facilitate reading or watching TV while in the bed 302, whereas in the evening, the bed 302 can be adjusted to a flat position to facilitate falling asleep). As another example, if the control circuitry 334 detects that the user 308 got out of bed at 3:00 am, the control circuitry 334 can use identified patterns for the user 308 to determine the user has gotten up temporarily (e.g., to use the bathroom, get a glass of water). The control circuitry 334 can turn on underbed lighting to assist the user 308 in carefully moving around the bed 302 and room. By contrast, if the control circuitry 334 identifies that the user 308 got out of the bed 302 at 6:40 am, the control circuitry 334 can determine the user 308 is up for the day and generate a different set of control signals (e.g., the control circuitry 334 can turn on light 326 near the bed 302 and/or raise the window blinds 330). For other users, getting out of the bed 302 at 3:00 am can be a normal wake-up time, which the control circuitry 334 can learn and respond to accordingly. Moreover, if the bed 302 is occupied by two users, the control circuitry 334 can learn and respond to the patterns of each of the users.

The bed 302 can also generate control signals based on communication with one or more devices. As an illustrative example, the control circuitry 334 can receive an indication from the television 312 that the television 312 is turned on. If the television 312 is located in a different room than the bed 302, the control circuitry 334 can generate a control signal to turn the television 312 off upon making a determination that the user 308 has gone to bed for the evening or otherwise is remaining in the room with the bed 302. If presence of the user 308 is detected on the bed 302 during a particular time range (e.g., between 8:00 pm and 7:00 am) and persists for longer than a threshold period of time (e.g., 10 minutes), the control circuitry 334 can determine the user 308 is in bed for the evening. If the television 312 is on, as described above, the control circuitry 334 can generate a control signal to turn the television 312 off. The control signals can be transmitted to the television (e.g., through a directed communication link or through a network, such as WIFI). As another example, rather than turning off the television 312 in response to detection of user bed presence, the control circuitry 334 can generate a control signal that causes the volume of the television 312 to be lowered by a pre-specified amount.

As another example, upon detecting that the user 308 has left the bed 302 during a specified time range (e.g., between 6:00 am and 8:00 am), the control circuitry 334 can generate control signals to cause the television 312 to turn on and tune to a pre-specified channel (e.g., the user 308 indicated a preference for watching morning news upon getting out of bed). The control circuitry 334 can accordingly generate and transmit the control signal to the television 312 (which can be stored at the control circuitry 334, the television 312, or another location). As another example, upon detecting that the user 308 has gotten up for the day, the control circuitry 334 can generate and transmit control signals to cause the television 312 to turn on and begin playing a previously recorded program from a digital video recorder (DVR) in communication with the television 312.

As another example, if the television 312 is in the same room as the bed 302, the control circuitry 334 may not cause the television 312 to turn off in response to detection of user bed presence. Rather, the control circuitry 334 can generate and transmit control signals to cause the television 312 to turn off in response to determining that the user 308 is asleep. For example, the control circuitry 334 can monitor biometric signals of the user 308 (e.g., motion, heartrate, respiration rate) to determine that the user 308 has fallen asleep. Upon detecting that the user 308 is sleeping, the control circuitry 334 generates and transmits a control signal to turn the television 312 off. As another example, the control circuitry 334 can generate the control signal to turn off the television 312 after a threshold period of time has passed since the user 308 has fallen asleep (e.g., 10 minutes after the user has fallen asleep). As another example, the control circuitry 334 generates control signals to lower the volume of the television 312 after determining that the user 308 is asleep. As yet another example, the control circuitry 334 generates and transmits a control signal to cause the television to gradually lower in volume over a period of time and then turn off in response to determining that the user 308 is asleep. Any of the control signals described above in reference to the television 312 can also be determined by the central controller previously described.

In some implementations, the control circuitry 334 can similarly interact with other media devices, such as computers, tablets, mobile phones, smart phones, wearable devices, stereo systems, etc. For example, upon detecting that the user 308 is asleep, the control circuitry 334 can generate and transmit a control signal to the user device 310 to cause the user device 310 to turn off, or turn down the volume on a video or audio file being played by the user device 310.

The control circuitry 334 can additionally communicate with the lighting system 314, receive information from the lighting system 314, and generate control signals for controlling functions of the lighting system 314. For example, upon detecting user bed presence on the bed 302 during a certain time frame (e.g., between 8:00 pm and 7:00 am) that lasts for longer than a threshold period of time (e.g., 10 minutes), the control circuitry 334 of the bed 302 can determine that the user 308 is in bed for the evening and generate control signals to cause lights in one or more rooms other than the room in which the bed 302 is located to switch off. The control circuitry 334 can generate and transmit control signals to turn off lights in all common rooms, but not in other bedrooms. As another example, the control signals can indicate that lights in all rooms other than the room in which the bed 302 is located are to be turned off, while one or more lights located outside of the house containing the bed 302 are to be turned on. The control circuitry 334 can generate and transmit control signals to cause the nightlight 328 to turn on in response to determining user 308 bed presence or that the user 308 is asleep. The control circuitry 334 can also generate first control signals for turning off a first set of lights (e.g., lights in common rooms) in response to detecting user bed presence, and second control signals for turning off a second set of lights (e.g., lights in the room where the bed 302 is located) when detecting that the user 308 is asleep.

In some implementations, in response to determining that the user 308 is in bed for the evening, the control circuitry 334 of the bed 302 can generate control signals to cause the lighting system 314 to implement a sunset lighting scheme in the room in which the bed 302 is located. A sunset lighting scheme can include, for example, dimming the lights (either gradually over time, or all at once) in combination with changing the color of the light in the bedroom environment, such as adding an amber hue to the lighting in the bedroom. The sunset lighting scheme can help to put the user 308 to sleep when the control circuitry 334 has determined that the user 308 is in bed for the evening. Sometimes, the control signals can cause the lighting system 314 to dim the lights or change color of the lighting in the bedroom environment, but not both.

The control circuitry 334 can also implement a sunrise lighting scheme when the user 308 wakes up in the morning. The control circuitry 334 can determine that the user 308 is awake for the day, for example, by detecting that the user 308 has gotten off the bed 302 (e.g., is no longer present on the bed 302) during a specified time frame (e.g., between 6:00 am and 8:00 am). The control circuitry 334 can also monitor movement, heartrate, respiratory rate, or other biometric signals of the user 308 to determine that the user 308 is awake or is waking up, even though the user 308 has not gotten out of bed. If the control circuitry 334 detects that the user is awake or waking up during a specified timeframe, the control circuitry 334 can determine that the user 308 is awake for the day. The specified timeframe can be, for example, based on previously recorded user bed presence information collected over a period of time (e.g., two weeks) that indicates that the user 308 usually wakes up for the day between 6:30 am and 7:30 am. In response to the control circuitry 334 determining that the user 308 is awake, the control circuitry 334 can generate control signals to cause the lighting system 314 to implement the sunrise lighting scheme in the bedroom in which the bed 302 is located. The sunrise lighting scheme can include, for example, turning on lights (e.g., the lamp 326, or other lights in the bedroom). The sunrise lighting scheme can further include gradually increasing the level of light in the room where the bed 302 is located (or in one or more other rooms). The sunrise lighting scheme can also include only turning on lights of specified colors. The sunrise lighting scheme can include lighting the bedroom with blue light to gently assist the user 308 in waking up and becoming active.

The control circuitry 334 may also generate different control signals for controlling actions of components depending on a time of day that user interactions with the bed 302 are detected. For example, the control circuitry 334 can use historical user interaction information to determine that the user 308 usually falls asleep between 10:00 pm and 11:00 pm and usually wakes up between 6:30 am and 7:30 am on weekdays. The control circuitry 334 can use this information to generate a first set of control signals for controlling the lighting system 314 if the user 308 is detected as getting out of bed at 3:00 am (e.g., turn on lights that guide the user 308 to a bathroom or kitchen) and to generate a second set of control signals for controlling the lighting system 314 if the user 308 is detected as getting out of bed after 6:30 am.

In some implementations, if the user 308 is detected as getting out of bed prior to a specified morning rise time for the user 308, the control circuitry 334 can cause the lighting system 314 to turn on lights that are dimmer than lights that are turned on by the lighting system 314 if the user 308 is detected as getting out of bed after the specified morning rise time. Causing the lighting system 314 to only turn on dim lights when the user 308 gets out of bed during the night (e.g., prior to normal rise time for the user 308) can prevent other occupants of the house from being woken up by the lights while still allowing the user 308 to see in order to reach their destination in the house.

The historical user interaction information for interactions between the user 308 and the bed 302 can be used to identify user sleep and awake timeframes. For example, user bed presence times and sleep times can be determined for a set period of time (e.g., two weeks, a month, etc.). The control circuitry 334 can identify a typical time range or timeframe in which the user 308 goes to bed, a typical timeframe for when the user 308 falls asleep, and a typical timeframe for when the user 308 wakes up (and in some cases, different timeframes for when the user 308 wakes up and when the user 308 actually gets out of bed). Buffer time may be added to these timeframes. For example, if the user is identified as typically going to bed between 10:00 pm and 10:30 pm, a buffer of a half hour in each direction can be added to the timeframe such that any detection of the user getting in bed between 9:30 pm and 11:00 pm is interpreted as the user 308 going to bed for the evening. As another example, detection of bed presence of the user 308 starting from a half hour before the earliest typical time that the user 308 goes to bed extending until the typical wake up time (e.g., 6:30 am) for the user 308 can be interpreted as the user 308 going to bed for the evening. For example, if the user 308 typically goes to bed between 10:00 pm and 10:30 pm, if the user 308's bed presence is sensed at 12:30 am one night, that can be interpreted as the user 308 getting into bed for the evening even though this is outside of the user 308's typical timeframe for going to bed because it has occurred prior to the user 308's normal wake up time. In some implementations, different timeframes are identified for different times of year (e.g., earlier bed time during winter vs. summer) or at different times of the week (e.g., user 308 wakes up earlier on weekdays than on weekends).

The control circuitry 334 can distinguish between the user 308 going to bed for an extended period (e.g., for the night) as opposed to being present on the bed 302 for a shorter period (e.g., for a nap) by sensing duration of presence of the user 308 (e.g., by detecting pressure and/or temperature signals of the user 308 on the bed 302 by sensors integrated into the bed 302). In some examples, the control circuitry 334 can distinguish between the user 308 going to bed for an extended period (e.g., for the night) versus going to bed for a shorter period (e.g., for a nap) by sensing duration of the user 308's sleep. The control circuitry 334 can set a time threshold whereby if the user 308 is sensed on the bed 302 for longer than the threshold, the user 308 is considered to have gone to bed for the night. In some examples, the threshold can be about 2 hours, whereby if the user 308 is sensed on the bed 302 for greater than 2 hours, the control circuitry 334 registers that as an extended sleep event. In other examples, the threshold can be greater than or less than two hours. The threshold can be determined based on historic trends indicating how long the user 302 usually sleeps or otherwise stays on the bed 302.

The control circuitry 334 can detect repeated extended sleep events to automatically determine a typical bed time range of the user 308, without requiring the user 308 to enter a bed time range. This can allow the control circuitry 334 to accurately estimate when the user 308 is likely to go to bed for an extended sleep event, regardless of whether the user 308 typically goes to bed using a traditional sleep schedule or a non-traditional sleep schedule. The control circuitry 334 can then use knowledge of the bed time range of the user 308 to control one or more components (including components of the bed 302 and/or non-bed peripherals) based on sensing bed presence during the bed time range or outside of the bed time range.

The control circuitry 334 can automatically determine the bed time range of the user 308 without requiring user inputs. The control circuitry 334 may also determine the bed time range automatically and in combination with user inputs (e.g., using signals sensed by sensors of the bed 302 and/or the central controller). The control circuitry 334 can set the bed time range directly according to user inputs. The control circuitry 334 can associate different bed times with different days of the week. In each of these examples, the control circuitry 334 can control components (e.g., the lighting system 314, thermostat 316, security system 318, oven 322, coffee maker 324, lamp 326, nightlight 328), as a function of sensed bed presence and the bed time range.

The control circuitry 334 can also determine control signals to be transmitted to the thermostat 316 based on user-inputted preferences and/or maintaining improved or preferred sleep quality of the user 308. For example, the control circuitry 334 can determine, based on historic sleep patterns and quality of the user 308 and by applying machine learning models, that the user 308 experiences their best sleep when the bedroom is at 74 degrees. The control circuitry 334 can receive temperature signals from devices and/or sensors in the bedroom indicating a bedroom temperature. When the temperature is below 74 degrees, the control circuitry 334 can determine control signals that cause the thermostat 316 to activate a heating unit to raise the temperature to 74 degrees in the bedroom. When the temperature is above 74 degrees, the control circuitry 334 can determine control signals that cause the thermostat 316 to activate a cooling unit to lower the temperature back to 74 degrees. Sometimes, the control circuitry 334 can determine control signals that cause the thermostat 316 to maintain the bedroom within a temperature range intended to keep the user 308 in particular sleep states and/or transition to next preferred sleep states.

Similarly, the control circuitry 334 can generate control signals to cause heating or cooling elements on the surface of the bed 302 to change temperature at various times, either in response to user interaction with the bed 302, at various pre-programmed times, based on user preference, and/or in response to detecting microclimate temperatures of the user 308 on the bed 302. For example, the control circuitry 334 can activate a heating element to raise the temperature of one side of the surface of the bed 302 to 73 degrees when it is detected that the user 308 has fallen asleep. As another example, upon determining that the user 308 is up for the day, the control circuitry 334 can turn off a heating or cooling element. The user 308 can pre-program various times at which the temperature at the bed surface should be raised or lowered. As another example, temperature sensors on the bed surface can detect microclimates of the user 308. When a detected microclimate drops below a predetermined threshold temperature, the control circuitry 334 can activate a heating element to raise the user 308's body temperature, thereby improving the user 308's comfortability, maintaining their sleep cycle, transitioning the user 308 to a next preferred sleep state, and/or maintaining or improving the user 308's sleep quality.

In response to detecting user bed presence and/or that the user 308 is asleep, the control circuitry 334 can also cause the thermostat 316 to change the temperature in different rooms to different values. Other control signals are also possible, and can be based on user preference and user input. Moreover, the control circuitry 334 can receive temperature information from the thermostat 316 and use this information to control functions of the bed 302 or other devices (e.g., adjusting temperatures of heating elements of the bed 302, such as a foot warming pad). The control circuitry 334 may also generate and transmit control signals for controlling other temperature control systems, such as floor heating elements in the bedroom or other rooms.

The control circuitry 334 can communicate with the security system 318, receive information from the security system 318, and generate control signals for controlling functions of the security system 318. For example, in response to detecting that the user 308 in is bed for the evening, the control circuitry 334 can generate control signals to cause the security system 318 to engage or disengage security functions. As another example, the control circuitry 334 can generate and transmit control signals to cause the security system 318 to disable in response to determining that the user 308 is awake for the day (e.g., user 308 is no longer present on the bed 302).

The control circuitry 334 can also receive alerts from the security system 318 and indicate the alert to the user 308. For example, the security system can detect a security breach (e.g., someone opened the door 332 without entering the security code, someone opened a window when the security system 318 is engaged) and communicate the security breach to the control circuitry 334. The control circuitry 334 can then generate control signals to alert the user 308, such as causing the bed 302 to vibrate, causing portions of the bed 302 to articulate (e.g., the head section to raise or lower), causing the lamp 326 to flash on and off at regular intervals, etc. The control circuitry 334 can also alert the user 308 of one bed 302 about a security breach in another bedroom, such as an open window in a kid's bedroom. The control circuitry 334 can send an alert to a garage door controller (e.g., to close and lock the door). The control circuitry 334 can send an alert for the security to be disengaged. The control circuitry 334 can also set off a smart alarm or other alarm device/clock near the bed 302. The control circuitry 334 can transmit a push notification, text message, or other indication of the security breach to the user device 310. Also, the control circuitry 334 can transmit a notification of the security breach to the central controller, which can then determine one or more responses to the security breach.

The control circuitry 334 can additionally generate and transmit control signals for controlling the garage door 320 and receive information indicating a state of the garage door 320 (e.g., open or closed). The control circuitry 334 can also request information on a current state of the garage door 320. If the control circuitry 334 receives a response (e.g., from the garage door opener) that the garage door 320 is open, the control circuitry 334 can notify the user 308 that the garage door is open (e.g., by displaying a notification or other message at the user device 310, outputting a notification at the central controller), and/or generate a control signal to cause the garage door opener to close the door. The control circuitry 334 can also cause the bed 302 to vibrate, cause the lighting system 314 to flash lights in the bedroom, etc. Control signals can also vary depend on the age of the user 308. Similarly, the control circuitry 334 can similarly send and receive communications for controlling or receiving state information associated with the door 332 or the oven 322.

In some implementations, different alerts can be generated for different events. For example, the control circuitry 334 can cause the lamp 326 (or other lights, via the lighting system 314) to flash in a first pattern if the security system 318 has detected a breach, flash in a second pattern if garage door 320 is on, flash in a third pattern if the door 332 is open, flash in a fourth pattern if the oven 322 is on, and flash in a fifth pattern if another bed has detected that a user 308 of that bed has gotten up (e.g., a child has gotten out of bed in the middle of the night as sensed by a sensor in the child's bed). Other examples of alerts include a smoke detector detecting smoke (and communicating this detection to the control circuitry 334), a carbon monoxide tester, a heater malfunctioning, or an alert from another device capable of communicating with the control circuitry 334 and detecting an occurrence to bring to the user 308's attention.

The control circuitry 334 can also communicate with a system or device for controlling a state of the window blinds 330. For example, in response to determining that the user 308 is up for the day or that the user 308 set an alarm to wake up at a particular time, the control circuitry 334 can generate and transmit control signals to cause the window blinds 330 to open. By contrast, if the user 308 gets out of bed prior to a normal rise time for the user 308, the control circuitry 334 can determine that the user 308 is not awake for the day and may not generate control signals that cause the window blinds 330 to open. The control circuitry 334 can also generate and transmit control signals that cause a first set of blinds to close in response to detecting user bed presence and a second set of blinds to close in response to detecting that the user 308 is asleep.

As other examples, in response to determining that the user 308 is awake for the day, the control circuitry 334 can generate and transmit control signals to the coffee maker 324 to cause the coffee maker 324 to brew coffee. The control circuitry 334 can generate and transmit control signals to the oven 322 to cause the oven 322 to begin preheating. The control circuitry 334 can use information indicating that the user 308 is awake for the day along with information indicating that the time of year is currently winter and/or that the outside temperature is below a threshold value to generate and transmit control signals to cause a car engine block heater to turn on. The control circuitry 334 can generate and transmit control signals to cause devices to enter a sleep mode in response to detecting user bed presence, or in response to detecting that the user 308 is asleep (e.g., causing a mobile phone of the user 308 to switch into sleep or night mode so that notifications are muted to not disturb the user 308's sleep). Later, upon determining that the user 308 is up for the day, the control circuitry 334 can generate and transmit control signals to cause the mobile phone to switch out of sleep/night mode.

The control circuitry 334 can also communicate with one or more noise control devices. For example, upon determining that the user 308 is in bed for the evening, or that the user 308 is asleep (e.g., based on pressure signals received from the bed 302, audio/decibel signals received from audio sensors positioned on or around the bed 302), the control circuitry 334 can generate and transmit control signals to cause noise cancelation devices to activate. The noise cancelation devices can be part of the bed 302 or located in the bedroom. Upon determining that the user 308 is in bed for the evening or that the user 308 is asleep, the control circuitry 334 can generate and transmit control signals to turn the volume on, off, up, or down, for one or more sound generating devices, such as a stereo system radio, television, computer, tablet, mobile phone, etc.

Additionally, functions of the bed 302 can be controlled by the control circuitry 334 in response to user interactions. For example, the articulation controller can adjust the bed 302 from a flat position to a position in which a head portion of a mattress of the bed 302 is inclined upward (e.g., to facilitate a user sitting up in bed, reading, and/or watching television). Sometimes, the bed 302 includes multiple separately articulable sections. Portions of the bed corresponding to the locations of the air chambers 306a and 306b can be articulated independently from each other, to allow one person to rest in a first position (e.g., a flat position) while a second person rests in a second position (e.g., a reclining position with the head raised at an angle from the waist). Separate positions can be set for two different beds (e.g., two twin beds placed next to each other). The foundation of the bed 302 can include more than one zone that can be independently adjusted. The articulation controller can also provide different levels of massage to one or more users on the bed 302 or cause the bed to vibrate to communicate alerts to the user 308 as described above.

The control circuitry 334 can adjust positions (e.g., incline and decline positions for the user 308 and/or an additional user) in response to user interactions with the bed 302 (e.g., causing the articulation controller to adjust to a first recline position in response to sensing user bed presence). The control circuitry 334 can cause the articulation controller to adjust the bed 302 to a second recline position (e.g., a less reclined, or flat position) in response to determining that the user 308 is asleep. As another example, the control circuitry 334 can receive a communication from the television 312 indicating that the user 308 has turned off the television 312, and in response, the control circuitry 334 can cause the articulation controller to adjust the bed position to a preferred user sleeping position (e.g., due to the user turning off the television 312 while the user 308 is in bed indicating the user 308 wishes to go to sleep).

In some implementations, the control circuitry 334 can control the articulation controller to wake up one user without waking another user of the bed 302. For example, the user 308 and a second user can each set distinct wakeup times (e.g., 6:30 am and 7:15 am respectively). When the wakeup time for the user 308 is reached, the control circuitry 334 can cause the articulation controller to vibrate or change the position of only a side of the bed on which the user 308 is located. When the wakeup time for the second user is reached, the control circuitry 334 can cause the articulation controller to vibrate or change the position of only the side of the bed on which the second user is located. Alternatively, when the second wakeup time occurs, the control circuitry 334 can utilize other methods (such as audio alarms, or turning on the lights) to wake the second user since the user 308 is already awake and therefore will not be disturbed when the control circuitry 334 attempts to wake the second user.

Still referring to FIG. 3, the control circuitry 334 for the bed 302 can utilize information for interactions with the bed 302 by multiple users to generate control signals for controlling functions of various other devices. For example, the control circuitry 334 can wait to generate control signals for devices until both the user 308 and a second user are detected in the bed 302. The control circuitry 334 can generate a first set of control signals to cause the lighting system 314 to turn off a first set of lights upon detecting bed presence of the user 308 and generate a second set of control signals for turning off a second set of lights in response to detecting bed presence of a second user. The control circuitry 334 can also wait until it has been determined that both users are awake for the day before generating control signals to open the window blinds 330. One or more other home automation control signals can be determined and generated by the control circuitry 334, the user device 310, and/or the central controller.

Examples of Data Processing Systems Associated with a Bed

Described are example systems and components for data processing tasks that are, for example, associated with a bed. In some cases, multiple examples of a particular component or group of components are presented. Some examples are redundant and/or mutually exclusive alternatives. Connections between components are shown as examples to illustrate possible network configurations for allowing communication between components. Different formats of connections can be used as technically needed/desired. The connections generally indicate a logical connection that can be created with any technologically feasible format. For example, a network on a motherboard can be created with a printed circuit board, wireless data connections, and/or other types of network connections. Some logical connections are not shown for clarity (e.g., connections with power supplies and/or computer readable memory).

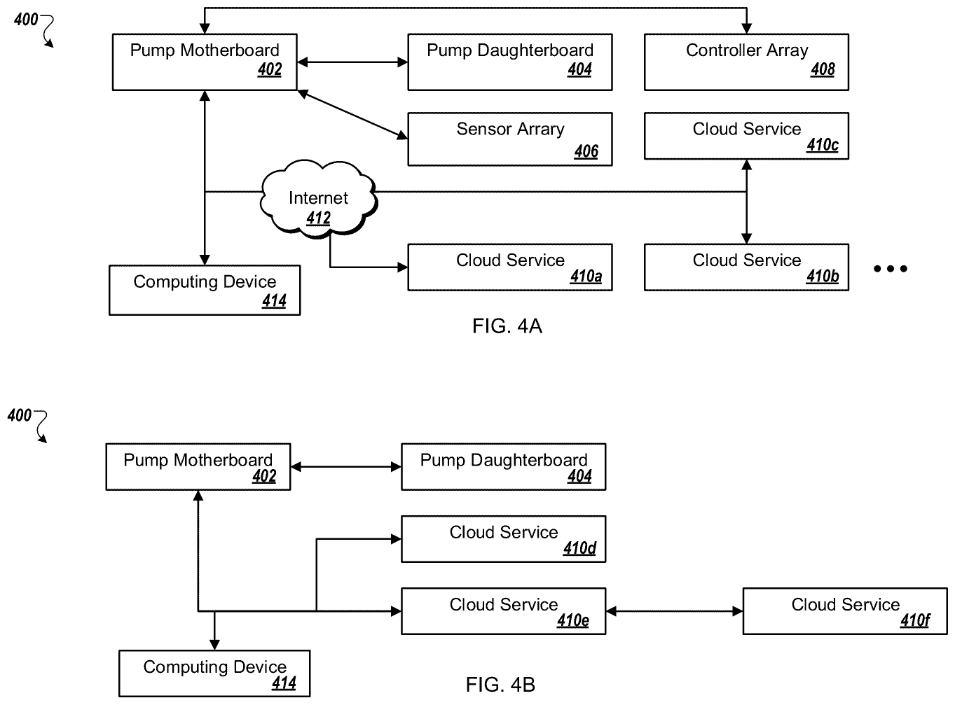

FIG. 4A is a block diagram of an example data processing system 400 that can be associated with a bed system, including those described above (e.g., see FIGS. 1-3). The system 400 includes a pump motherboard 402 and a pump daughterboard 404. The system 400 includes a sensor array 406 having one or more sensors configured to sense physical phenomenon of the environment and/or bed, and to report sensing back to the pump motherboard 402 (e.g., for analysis). The sensor array 406 can include one or more different types of sensors, including but not limited to pressure, temperature, light, movement (e.g. motion), and audio. The system 400 also includes a controller array 408 that can include one or more controllers configured to control logic-controlled devices of the bed and/or environment (e.g., home automation devices, security systems light systems, and other devices described in FIG. 3). The pump motherboard 400 can be in communication with computing devices 414 and cloud services 410 over local networks (e.g., Internet 412) or otherwise as is technically appropriate.

In FIG. 4A, the pump motherboard 402 and daughterboard 404 are communicably coupled. They can be conceptually described as a center or hub of the system 400, with the other components conceptually described as spokes of the system 400. This can mean that each spoke component communicates primarily or exclusively with the pump motherboard 402. For example, a sensor of the sensor array 406 may not be configured to, or may not be able to, communicate directly with a corresponding controller. Instead, the sensor can report a sensor reading to the motherboard 402, and the motherboard 402 can determine that, in response, a controller of the controller array 408 should adjust some parameters of a logic controlled device or otherwise modify a state of one or more peripheral devices.

One advantage of a hub-and-spoke network configuration, or a star-shaped network, is a reduction in network traffic compared to, for example, a mesh network with dynamic routing. If a particular sensor generates a large, continuous stream of traffic, that traffic is transmitted over one spoke to the motherboard 402. The motherboard 402 can marshal and condense that data to a smaller data format for retransmission for storage in a cloud service 410. Additionally or alternatively, the motherboard 402 can generate a single, small, command message to be sent down a different spoke in response to the large stream. For example, if the large stream of data is a pressure reading transmitted from the sensor array 406 a few times a second, the motherboard 402 can respond with a single command message to the controller array 408 to increase the pressure in an air chamber of the bed. In this case, the single command message can be orders of magnitude smaller than the stream of pressure readings.

As another advantage, a hub-and-spoke network configuration can allow for an extensible network that accommodates components being added, removed, failing, etc. This can allow more, fewer, or different sensors in the sensor array 406, controllers in the controller array 408, computing devices 414, and/or cloud services 410. For example, if a particular sensor fails or is deprecated by a newer version, the system 400 can be configured such that only the motherboard 402 needs to be updated about the replacement sensor. This can allow product differentiation where the same motherboard 402 can support an entry level product with fewer sensors and controllers, a higher value product with more sensors and controllers, and customer personalization where a customer can add their own selected components to the system 400.

Additionally, a line of air bed products can use the system 400 with different components. In an application in which every air bed in the product line includes both a central logic unit and a pump, the motherboard 402 (and optionally the daughterboard 404) can be designed to fit within a single, universal housing. For each upgrade of the product in the product line, additional sensors, controllers, cloud services, etc., can be added. Design, manufacturing, and testing time can be reduced by designing all products in a product line from this base, compared to a product line in which each product has a bespoke logic control system.

Each of the components discussed above can be realized in a wide variety of technologies and configurations. Below, some examples of each component are discussed. Sometimes, two or more components of the system 400 can be realized in a single alternative component; some components can be realized in multiple, separate components; and/or some functionality can be provided by different components.

FIG. 4B is a block diagram showing communication paths of the system 400. As described, the motherboard 402 and daughterboard 404 may act as a hub of the system 400. When the pump daughterboard 404 communicates with cloud services 410 or other components, communications may be routed through the motherboard 402. This may allow the bed to have a single connection with the Internet 412. The computing device 414 may also have a connection to the Internet 412, possibly through the same gateway used by the bed and/or a different gateway (e.g., a cell service provider).

In FIG. 4B, cloud services 410d and 410e may be configured such that the motherboard 402 communicates with the cloud service directly (e.g., without having to use another cloud service 410 as an intermediary). Additionally or alternatively, some cloud services 410 (e.g., 410f) may only be reachable by the motherboard 402 through an intermediary cloud service (e.g., 410e). While not shown here, some cloud services 410 may be reachable either directly or indirectly by the pump motherboard 402.

Additionally, some or all of the cloud services 410 may communicate with other cloud services, including the transfer of data and/or remote function calls according to any technologically appropriate format. For example, one cloud service 410 may request a copy for another cloud service's 410 data (e.g., for purposes of backup, coordination, migration, calculations, data mining). Many cloud services 410 may also contain data that is indexed according to specific users tracked by the user account cloud 410c and/or the bed data cloud 410a. These cloud services 410 may communicate with the user account cloud 410c and/or the bed data cloud 410a when accessing data specific to a particular user or bed.

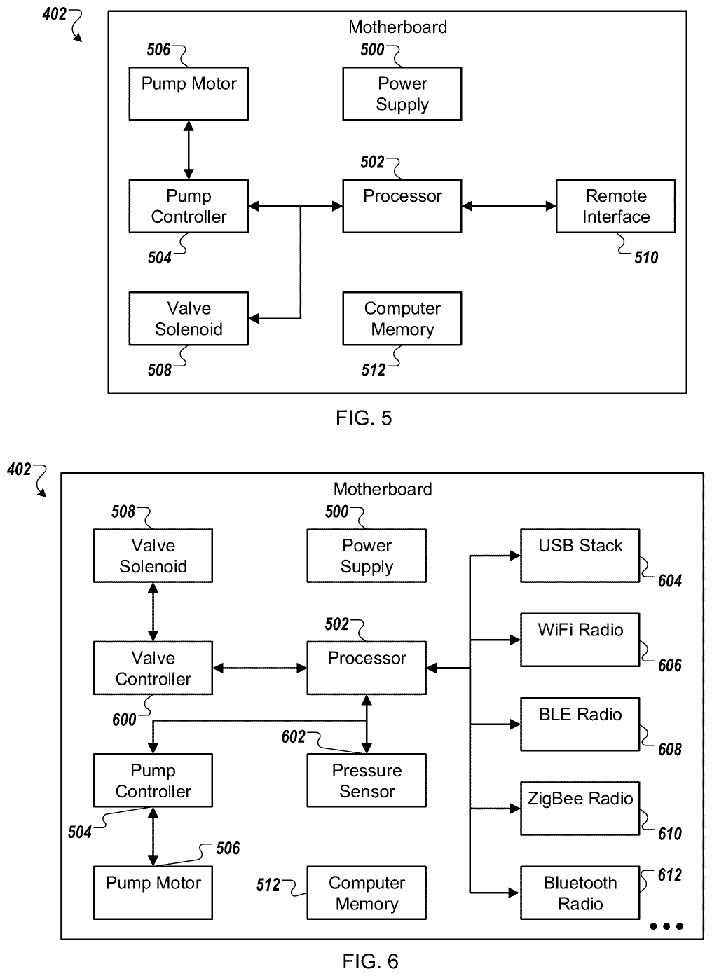

FIG. 5 is a block diagram of an example motherboard 402 in a data processing system associated with a bed system (e.g., refer to FIGS. 1-3). In this example, compared to other examples described below, this motherboard 402 consists of relatively fewer parts and can be limited to provide a relatively limited feature set.

The motherboard 402 includes a power supply 500, a processor 502, and computer memory 512. In general, the power supply 500 includes hardware used to receive electrical power from an outside source and supply it to components of the motherboard 402. The power supply may include a battery pack and/or wall outlet adapter, an AC to DC converter, a DC to AC converter, a power conditioner, a capacitor bank, and/or one or more interfaces for providing power in the current type, voltage, etc., needed by other components of the motherboard 402.

The processor 502 is generally a device for receiving input, performing logical determinations, and providing output. The processor 502 can be a central processing unit, a microprocessor, general purpose logic circuitry, application-specific integrated circuitry, a combination of these, and/or other hardware.

The memory 512 is generally one or more devices for storing data, which may include long term stable data storage (e.g., on a hard disk), short term unstable (e.g., on Random Access Memory), or any other technologically appropriate configuration.

The motherboard 402 includes a pump controller 504 and a pump motor 506. The pump controller 504 can receive commands from the processor 502 to control functioning of the pump motor 506. For example, the pump controller 504 can receive a command to increase pressure of an air chamber by 0.3 pounds per square inch (PSI). The pump controller 504, in response, engages a valve so that the pump motor 506 pumps air into the selected air chamber, and can engage the pump motor 506 for a length of time that corresponds to 0.3 PSI or until a sensor indicates that pressure has been increased by 0.3 PSI. Sometimes, the message can specify that the chamber should be inflated to a target PSI, and the pump controller 504 can engage the pump motor 506 until the target PSI is reached.

A valve solenoid 508 can control which air chamber a pump is connected to. In some cases, the solenoid 508 can be controlled by the processor 502 directly. In some cases, the solenoid 508 can be controlled by the pump controller 504.

A remote interface 510 of the motherboard 402 can allow the motherboard 402 to communicate with other components of a data processing system. For example, the motherboard 402 can be able to communicate with one or more daughterboards, with peripheral sensors, and/or with peripheral controllers through the remote interface 510. The remote interface 510 can provide any technologically appropriate communication interface, including but not limited to multiple communication interfaces such as WIFI, Bluetooth, and copper wired networks.

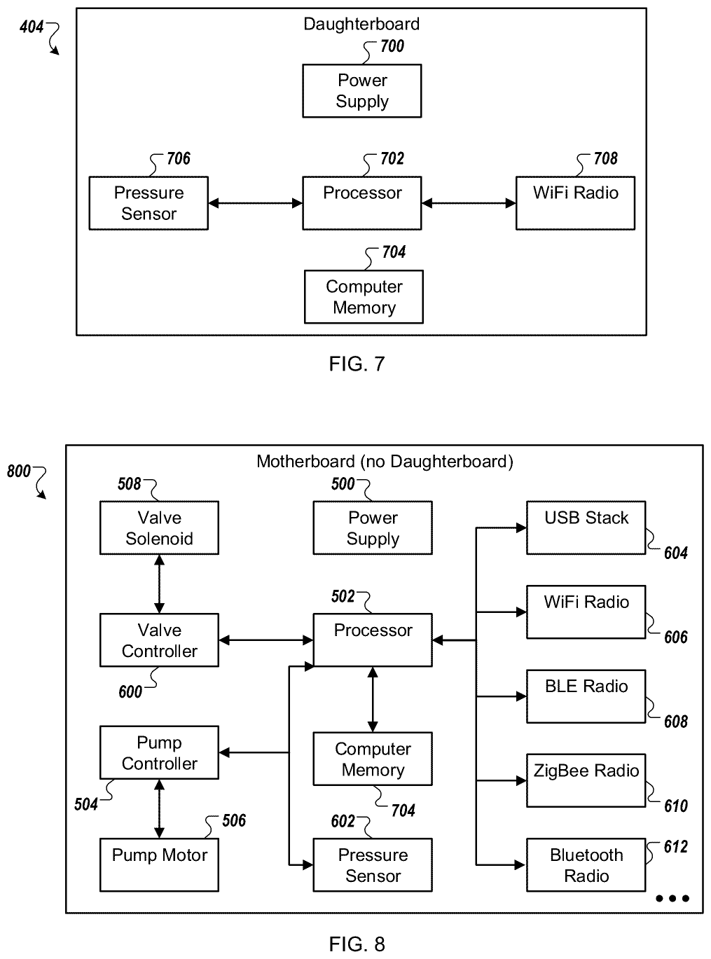

FIG. 6 is a block diagram of another example motherboard 402. Compared to the motherboard 402 in FIG. 5, the motherboard 402 in FIG. 6 can contain more components and provide more functionality in some applications.

This motherboard 402 can further include a valve controller 600, a pressure sensor 602, a universal serial bus (USB) stack 604, a WiFi radio 606, a Bluetooth Low Energy (BLE) radio 608, a ZigBee radio 610, a Bluetooth radio 612, and a computer memory 512.

The valve controller 600 can convert commands from the processor 502 into control signals for the valve solenoid 508. For example, the processor 502 can issue a command to the valve controller 600 to connect the pump to a particular air chamber out of a group of air chambers in an air bed. The valve controller 600 can control the position of the valve solenoid 508 so the pump is connected to the indicated air chamber.

The pressure sensor 602 can read pressure readings from one or more air chambers of the air bed. The pressure sensor 602 can also preform digital sensor conditioning. As described herein, multiple pressure sensors 602 can be included as part of the motherboard 402 or otherwise in communication with the motherboard 402.

The motherboard 402 can include a suite of network interfaces 604, 606, 608, 610, 612, etc., including but not limited to those shown in FIG. 6. These network interfaces can allow the motherboard to communicate over a wired or wireless network with any devices, including but not limited to peripheral sensors, peripheral controllers, computing devices, and devices and services connected to the Internet 412.

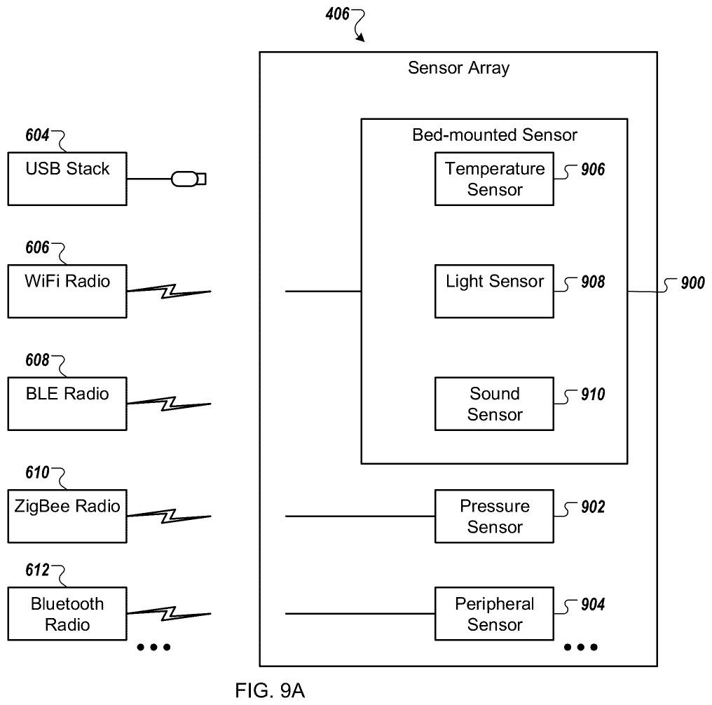

FIG. 7 is a block diagram of an example daughterboard 404 used in a data processing system associated with a bed system described herein. One or more daughterboards 404 can be connected to the motherboard 402. Some daughterboards 404 can be designed to offload particular and/or compartmentalized tasks from the motherboard 402. This can be advantageous if the particular tasks are computationally intensive, proprietary, or subject to future revisions. For example, the daughterboard 404 can be used to calculate a particular sleep data metric. This metric can be computationally intensive, and calculating the metric on the daughterboard 404 can free up resources of the motherboard 402 while the metric is calculated. The sleep metric may be subject to future revisions. To update the system 400 with the new metric, it is possible that only the daughterboard 404 calculates the metric to be replaced. In this case, the same motherboard 402 and other components can be used, saving the need to perform unit testing of additional components instead of just the daughterboard 404.

The daughterboard 404 includes a power supply 700, a processor 702, computer readable memory 704, a pressure sensor 706, and a WiFi radio 708. The processor 702 can use the pressure sensor 706 to gather information about pressure of air bed chambers. The processor 702 can perform an algorithm to calculate a sleep metric (e.g., sleep quality, bed presence, whether the user fell asleep, a heartrate, a respiration rate, movement, etc.). Sometimes, the sleep metric can be calculated from only air chamber pressure. The sleep metric can also be calculated using signals from a variety of sensors (e.g., movement, pressure, temperature, and/or audio sensors). The processor 702 can receive that data from sensors that may be internal to the daughterboard 404, accessible via the WiFi radio 708, or otherwise in communication with the processor 702. Once the sleep metric is calculated, the processor 702 can report that sleep metric to, for example, the motherboard 402. The motherboard 402 can generate instructions for outputting the sleep metric to the user or using the sleep metric to determine other user information or controls to control the bed and/or peripheral devices.

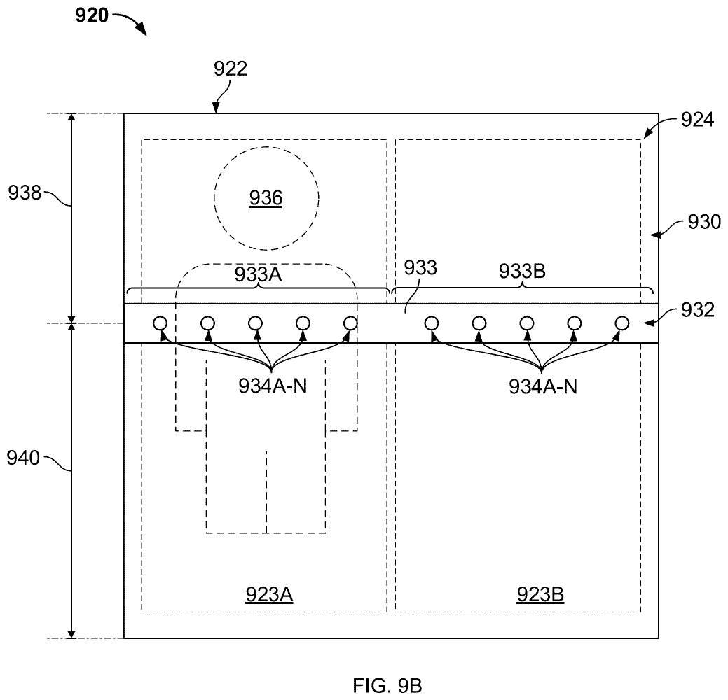

FIG. 8 is a block diagram of an example motherboard 800 with no daughterboard used in a data processing system associated with a bed system. In this example, the motherboard 800 can perform most, all, or more of the features described with reference to the motherboard 402 in FIG. 6 and the daughterboard 404 in FIG. 7.

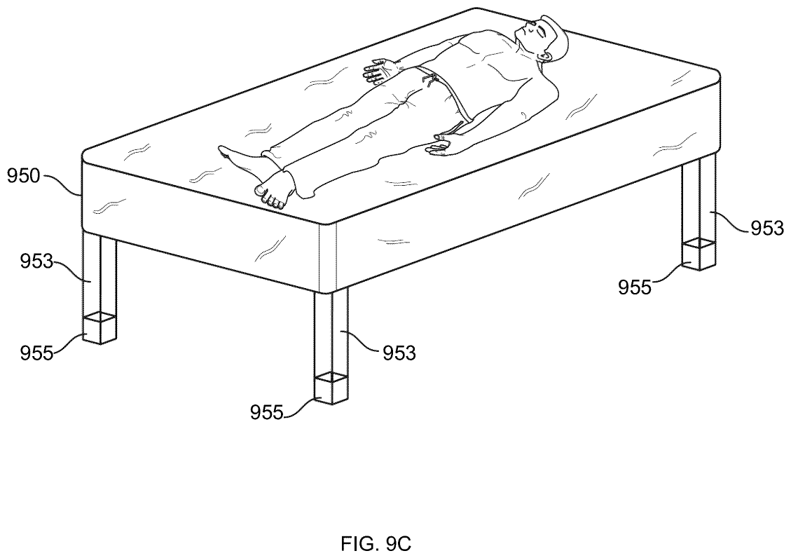

FIG. 9A is a block diagram of an example sensory array 406 used in a data processing system associated with a bed system described herein. The sensor array 406 is a conceptual grouping of some or all peripheral sensors that communicate with the motherboard 402 but are not native to the motherboard 402. The peripheral sensors 902, 904, 906, 908, 910, etc. of the sensor array 406 communicate with the motherboard 402 through one or more network interfaces 604, 606, 608, 610, and 612 of the motherboard, as is appropriate for the configuration of the particular sensor. For example, a sensor that outputs a reading over a USB cable can communicate through the USB stack 604.

Some peripheral sensors of the sensor array 406 can be bed mounted sensors 900 (e.g., temperature sensor 906, light sensor 908, sound sensor 910). The bed mounted sensors 900 can be embedded into a bed structure and sold with the bed, or later affixed to the structure (e.g., part of a pressure sensing pad that is removably installed on a top surface of the bed, part of a temperature sensing or heating pad that is removably installed on the top surface of the bed, integrated into the top surface, attached along connecting tubes between a pump and air chambers, within air chambers, attached to a headboard, attached to one or more regions of an adjustable foundation). One or more of the sensors 902 can be load cells or force sensors as described in FIG. 9C. Other sensors 902 and 904 may not be mounted to the bed and can include a pressure sensor 902 and/or peripheral sensor 904. For example, the sensors 902 and 904 can be integrated or otherwise part of a user mobile device (e.g., mobile phone, wearable device). The sensors 902 and 904 can also be part of a central controller for controlling the bed and peripheral devices. Sometimes, the sensors 902 and 904 can be part of one or more home automation devices or other peripheral devices.

Sometimes, some or all of the bed mounted sensors 900 and/or sensors 902 and 904 share networking hardware (e.g., a conduit that contains wires from each sensor, a multi-wire cable or plug that, when affixed to the motherboard 402, connect all the associated sensors with the motherboard 402). One, some, or all the sensors 902, 904, 906, 908, and 910 can sense features of a mattress (e.g., pressure, temperature, light, sound, and/or other features) and features external to the mattress. Sometimes, pressure sensor 902 can sense pressure of the mattress while some or all the sensors 902, 904, 906, 908, and 910 sense features of the mattress and/or features external to the mattress.

FIG. 9B is a schematic top view of a bed 920 having a sensor strip 932 with sensors 934A-N used in a data processing system associated with the bed 920. The bed 920 includes a mattress 922 (e.g., refer to FIG. 1). The mattress 922 can have a foam tub 930 beneath a top of the mattress 922. The foam tub 930 can have air chamber 923A and/or 923B, similar to those described herein.

The sensor strip 932 can be attached across the mattress top 924 from one lateral side to an opposing lateral side (e.g., from left to right). The sensor strip 932 can be attached proximate to a head section of the mattress 922 to measure temperature and/or humidity values around a chest area of a user 936. The sensor strip 932 can also be placed at a center point (e.g., midpoint) of the mattress 922 such that the distances 938 and 940 are equal to each other. The sensor strip 932 can be placed at other locations to capture temperature and/or humidity values at the top of the mattress 922.

The sensors 934A-N can be any one or more of the temperature sensors 906 described in FIG. 9A. The sensor strip 932 can also include a carrier strip 933 having a first strip portion 933A and a second strip portion 933B. The carrier strip 933 can be releasably attached to the foam tub layer 920 and extend between the opposite lateral ends of the foam tub 920. The sensor strip 932 can have first sensors 934A-N and second sensors 934A-N. Each of the first and second sensors 934A-N can have five sensors each. For example, a sensor strip 932 for a king or queen size mattress can have a total of ten sensors. When the user 936 is positioned on top of the mattress 922 over the air chamber 923A, the first sensors 934A-N can measure temperature and/or humidity of the mattress top 924 above the air chamber 923A. Those values can be used to, for example, determine a conditioned airflow to supply to the air chamber 923A. Temperature and/or humidity values measured by the second sensors 934A-N can be used to, for example, determine a conditioned airflow to supply to the air chamber 923B. The bed system 920 can provide for custom airflow to different portions of the mattress 922 based on body temperatures of users and/or temperatures of different portions of the mattress top 924.

Sometimes, two separate sensor strips can be attached to the mattress 922 (e.g., a first sensor strip over the air chamber 923A and a second sensor strip, separate from the first sensor strip, over the air chamber 923B). The first and second sensor strips can be attached to a center of the mattress top 924 via fastening elements, such as adhesive. The sensor strip 932 can also be easily replaced with another sensor strip.

FIG. 9C is a schematic diagram of an example bed with force sensors 955 located at the bottom of legs 953 of the bed (e.g., in four, six, eight, or another number of legs). The force sensors 955 may also be located elsewhere on the bed with similar effect (e.g., between the legs 953 and platform 950). When a strain gauge is used as the force sensors 955, the force sensor(s) 955 can be positioned nearer centers of the legs 953. The force sensors 955 can be load cells.

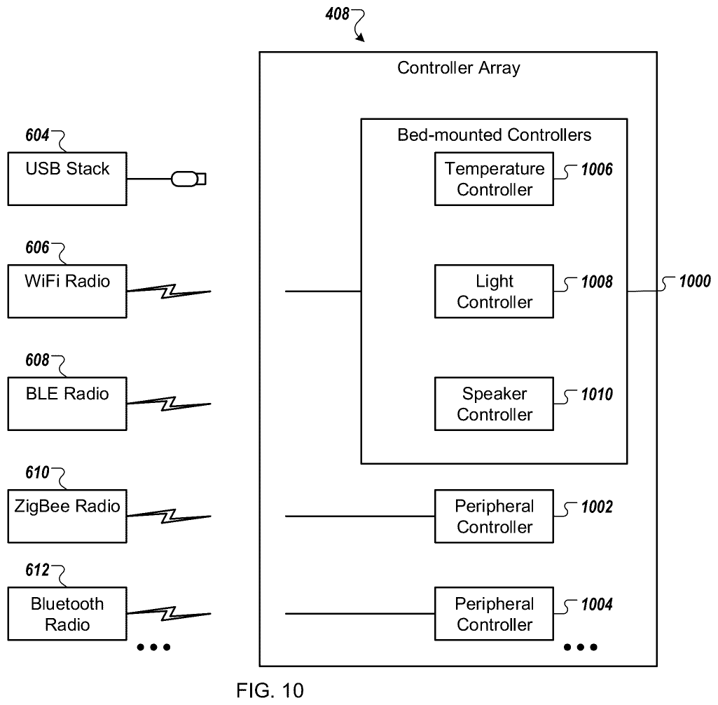

FIG. 10 is a block diagram of an example controller array 408 used in a data processing system associated with a bed system. The controller array 408 is a conceptual grouping of some or all peripheral controllers that communicate with the motherboard 402 but are not native to the motherboard 402. The peripheral controllers can communicate with the motherboard 402 through one or more of the network interfaces 604, 606, 608, 610, and 612 of the motherboard, as is appropriate for the configuration of the particular controller. Some of the controllers can be bed mounted controllers 1000, such as a temperature controller 1006, a light controller 1008, and a speaker controller 1010, as described in reference to bed-mounted sensors in FIG. 9A. Peripheral controllers 1002 and 1004 can be in communication with the motherboard 402, but optionally not mounted to the bed.

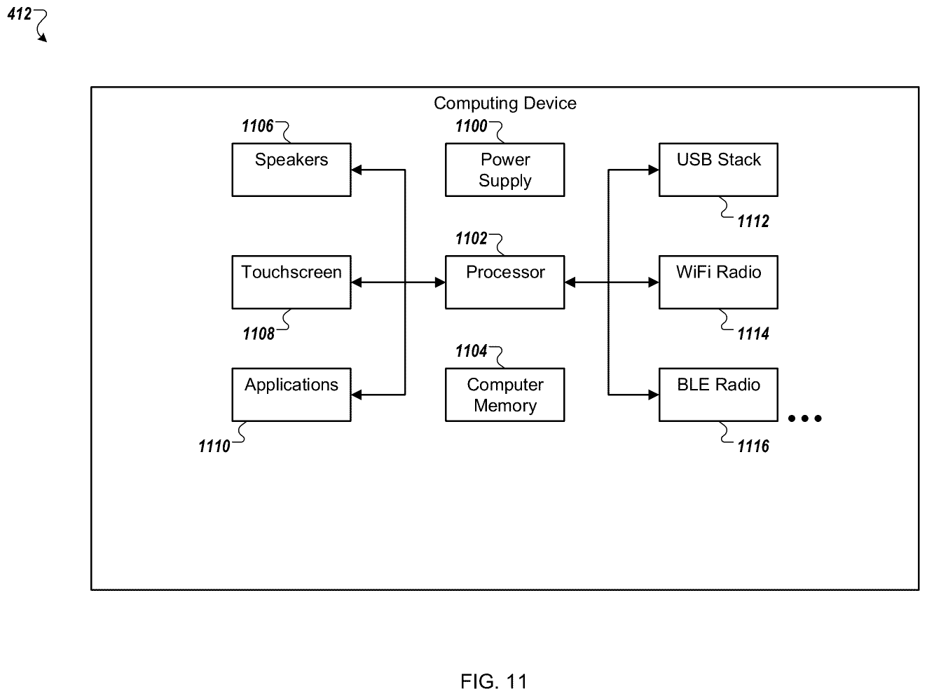

FIG. 11 is a block diagram of an example computing device 412 used in a data processing system associated with a bed system. The computing device 412 can include computing devices used by a user of a bed including but not limited to mobile computing devices (e.g., mobile phones, tablet computers, laptops, smart phones, wearable devices), desktop computers, home automation devices, and/or central controllers or other hub devices.

The computing device 412 includes a power supply 1100, a processor 1102, and computer readable memory 1104. User input and output can be transmitted by speakers 1106, a touchscreen 1108, or other not shown components (e.g., a pointing device or keyboard). The computing device 412 can run applications 1110 including, for example, applications to allow the user to interact with the system 400. These applications can allow a user to view information about the bed (e.g., sensor readings, sleep metrics), information about themselves (e.g., health conditions detected based on signals sensed at the bed), and/or configure the system 400 behavior (e.g., set desired firmness, set desired behavior for peripheral devices). The computing device 412 can be used in addition to, or to replace, the remote control 122 described above.

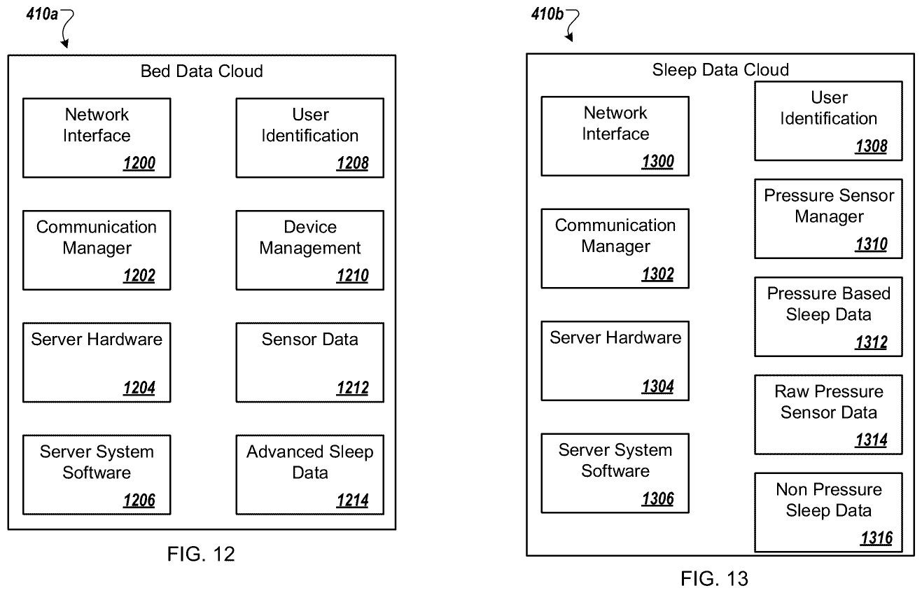

FIG. 12 is a block diagram of an example bed data cloud service 410a used in a data processing system associated with a bed system. Here, the bed data cloud service 410a is configured to collect sensor data and sleep data from a particular bed, and to match the data with one or more users that used the bed when the data was generated.

The bed data cloud service 410a includes a network interface 1200, a communication manager 1202, server hardware 1204, and server system software 1206. The bed data cloud service 410a is also shown with a user identification module 1208, a device management 1210 module, a sensor data module 1210, and an advanced sleep data module 1214. The network interface 1200 includes hardware and low level software to allow hardware devices (e.g., components of the service 410a) to communicate over networks (e.g., with each other, with other destinations over the Internet 412). The network interface 1200 can include network cards, routers, modems, and other hardware. The communication manager 1202 generally includes hardware and software that operate above the network interface 1200 such as software to initiate, maintain, and tear down network communications used by the service 410a (e.g., TCP/IP, SSL or TLS, Torrent, and other communication sessions over local or wide area networks). The communication manager 1202 can also provide load balancing and other services to other elements of the service 410a. The server hardware 1204 generally includes physical processing devices used to instantiate and maintain the service 410a. This hardware includes, but is not limited to, processors (e.g., central processing units, ASICs, graphical processers) and computer readable memory (e.g., random access memory, stable hard disks, tape backup). One or more servers can be configured into clusters, multi-computer, or datacenters that can be geographically separate or connected. The server system software 1206 generally includes software that runs on the server hardware 1204 to provide operating environments to applications and services (e.g., operating systems running on real servers, virtual machines instantiated on real servers to create many virtual servers, server level operations such as data migration, redundancy, and backup).

The user identification 1208 can include, or reference, data related to users of beds with associated data processing systems. The users may include customers, owners, or other users registered with the service 410a or another service. Each user can have a unique identifier, user credentials, contact information, billing information, demographic information, or any other technologically appropriate information.

The device manager 1210 can include, or reference, data related to beds or other products associated with data processing systems. The beds can include products sold or registered with a system associated with the service 410a. Each bed can have a unique identifier, model and/or serial number, sales information, geographic information, delivery information, a listing of associated sensors and control peripherals, etc. An index or indexes stored by the service 410a can identify users associated with beds. This index can record sales of a bed to a user, users that sleep in a bed, etc.

The sensor data 1212 can record raw or condensed sensor data recorded by beds with associated data processing systems. For example, a bed's data processing system can have temperature, pressure, motion, audio, and/or light sensors. Readings from these sensors, either in raw form or in a format generated from the raw data (e.g. sleep metrics), can be communicated by the bed's data processing system to the service 410a for storage in the sensor data 1212. An index or indexes stored by the service 410a can identify users and/or beds associated with the sensor data 1212.

The service 410a can use any of its available data (e.g., sensor data 1212) to generate advanced sleep data 1214. The advanced sleep data 1214 includes sleep metrics and other data generated from sensor readings (e.g., health information). Some of these calculations can be performed in the service 410a instead of locally on the bed's data processing system because the calculations can be computationally complex or require a large amount of memory space or processor power that may not be available on the bed's data processing system. This can help allow a bed system to operate with a relatively simple controller while being part of a system that performs relatively complex tasks and computations.

For example, the service 410a can retrieve one or more machine learning models from a remote data store and use those models to determine the advanced sleep data 1214. The service 410a can retrieve one or more models to determine overall sleep quality of the user based on currently detected sensor data 1212 and/or historic sensor data. The service 410a can retrieve other models to determine whether the user is snoring based on the detected sensor data 1212. The service 410a can retrieve other models to determine whether the user experiences a health condition based on the data 1212.

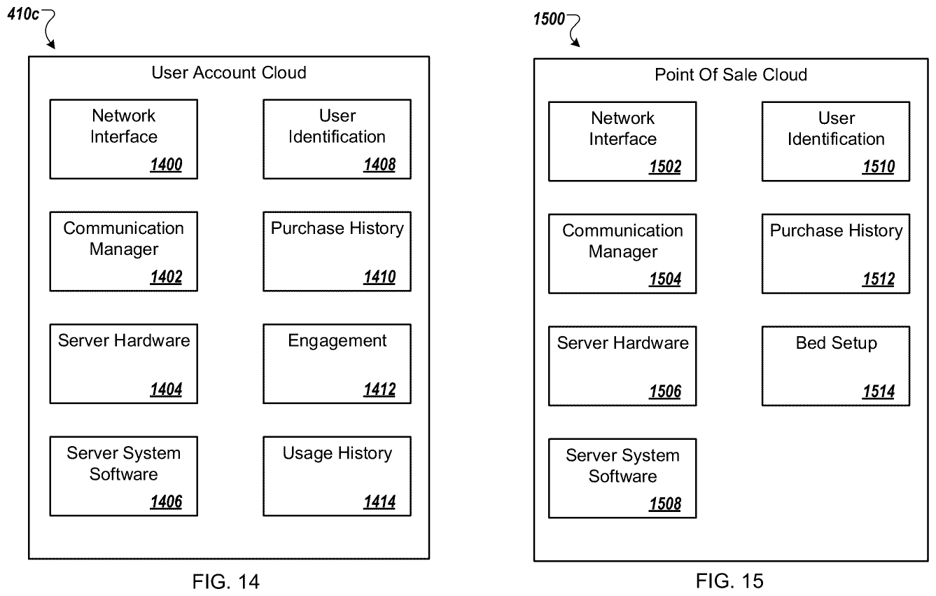

FIG. 13 is a block diagram of an example sleep data cloud service 410b used in a data processing system associated with a bed system. Here, the sleep data cloud service 410b is configured to record data related to users' sleep experience. The service 410b includes a network interface 1300, a communication manager 1302, server hardware 1304, and server system software 1306. The service 410b also includes a user identification module 1308, a pressure sensor manager 1310, a pressure based sleep data module 1312, a raw pressure sensor data module 1314, and a non-pressure sleep data module 1316. Sometimes, the service 410b can include a sensor manager for each sensor. The service 410b can also include a sensor manager that relates to multiple sensors in beds (e.g., a single sensor manager can relate to pressure, temperature, light, movement, and audio sensors in a bed).

The pressure sensor manager 1310 can include, or reference, data related to the configuration and operation of pressure sensors in beds. This data can include an identifier of the types of sensors in a particular bed, their settings and calibration data, etc. The pressure based sleep data 1312 can use raw pressure sensor data 1314 to calculate sleep metrics tied to pressure sensor data. For example, user presence, movements, weight change, heartrate, and breathing rate can be determined from raw pressure sensor data 1314. An index or indexes stored by the service 410b can identify users associated with pressure sensors, raw pressure sensor data, and/or pressure based sleep data. The non-pressure sleep data 1316 can use other sources of data to calculate sleep metrics. User-entered preferences, light sensor readings, and sound sensor readings can be used to track sleep data. User presence can also be determined from a combination of raw pressure sensor data 1314 and non-pressure sleep data 1316 (e.g., raw temperature data). Sometimes, bed presence can be determined using only the temperature data. Changes in temperature data can be monitored to determine bed presence or absence in a temporal interval (e.g., window of time) of a given duration. The temperature and/or pressure data can also be combined with other sensing modalities or motion sensors that reflect different forms of movement (e.g., load cells) to accurately detect user presence. For example, the temperature and/or pressure data can be provided as input to a bed presence classifier, which can determine user bed presence based on real-time or near real-time data collected at the bed. The classifier can be trained to differentiate the temperature data from the pressure data, identify peak values in the temperature and pressure data, and generate a bed presence indication based on correlating the peak values. The peak values can be within a threshold distance from each other to then generate an indication that the user is in the bed. An index or indexes stored by the service 410b can identify users associated with sensors and/or the data 1316.

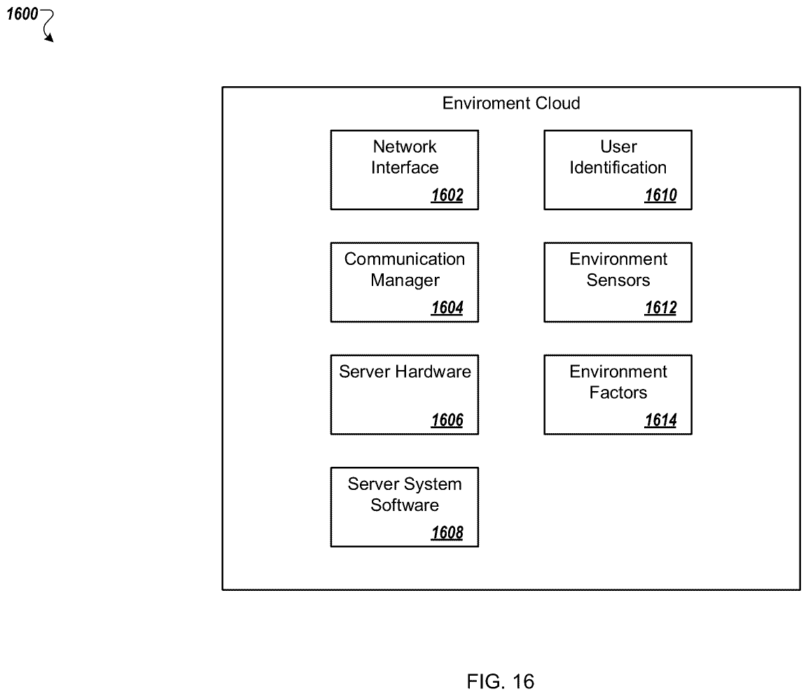

FIG. 14 is a block diagram of an example user account cloud service 410c used in a data processing system associated with a bed system. Here, the service 410c is configured to record a list of users and to identify other data related to those users. The service 410c includes a network interface 1400, a communication manager 1402, server hardware 1404, and server system software 1406. The service 410c also includes a user identification module 1408, a purchase history module 1410, an engagement module 1412, and an application usage history module 1414.

The user identification module 1408 can include, or reference, data related to users of beds with associated data processing systems, as described above. The purchase history module 1410 can include, or reference, data related to purchases by users. The purchase data can include a sale's contact information, billing information, and salesperson information associated with the user's purchase of the bed system. An index or indexes stored by the service 410c can identify users associated with a bed purchase.

The engagement module 1412 can track user interactions with the manufacturer, vendor, and/or manager of the bed/cloud services. This data can include communications (e.g., emails, service calls), data from sales (e.g., sales receipts, configuration logs), and social network interactions. The data can also include servicing, maintenance, or replacements of components of the user's bed system. The usage history module 1414 can contain data about user interactions with applications and/or remote controls of the bed. A monitoring and configuration application can be distributed to run on, for example, computing devices 412 described herein. The application can log and report user interactions for storage in the application usage history module 1414. An index or indexes stored by the service 410c can also identify users associated with each log entry. User interactions stored in the module 1414 can optionally be used to determine or predict user preferences and/or settings for the user's bed and/or peripheral devices that can improve the user's overall sleep quality.

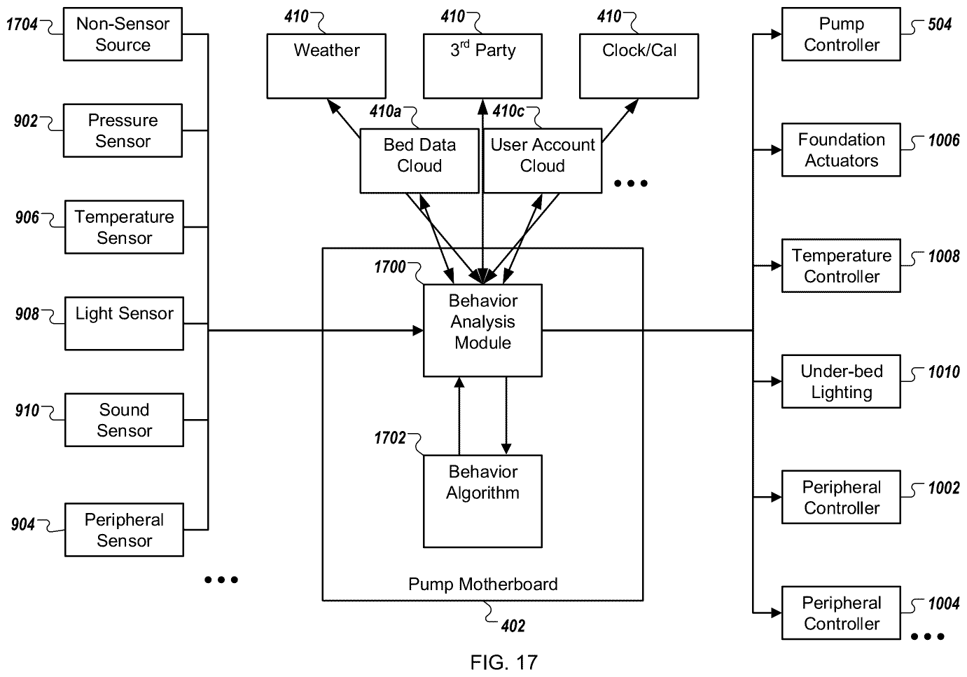

FIG. 15 is a block diagram of an example point of sale cloud service 1500 used in a data processing system associated with a bed system. Here, the service 1500 can record data related to users' purchases, specifically purchases of bed systems described herein. The service 1500 is shown with a network interface 1502, a communication manager 1504, server hardware 1506, and server system software 1508. The service 1500 also includes a user identification module 1510, a purchase history module 1512, and a bed setup module 1514.

The purchase history module 1512 can include, or reference, data related to purchases made by users identified in the module 1510, such as data of a sale, price, and location of sale, delivery address, and configuration options selected by the users at the time of sale. The configuration options can include selections made by the user about how they wish their newly purchased beds to be setup and can include expected sleep schedule, a listing of peripheral sensors and controllers that they have or will install, etc.

The bed setup module 1514 can include, or reference, data related to installations of beds that users purchase. The bed setup data can include a date and address to which a bed is delivered, a person who accepts delivery, configuration that is applied to the bed upon delivery (e.g., firmness settings), name(s) of bed user(s), which side of the bed each user will use, etc. Data recorded in the service 1500 can be referenced by a user's bed system at later times to control functionality of the bed system and/or to send control signals to peripheral components. This can allow a salesperson to collect information from the user at the point of sale that later facilitates bed system automation. Sometimes, some or all aspects of the bed system can be automated with little or no user-entered data required after the point of sale. Sometimes, data recorded in the service 1500 can be used in connection with other, user-entered data.

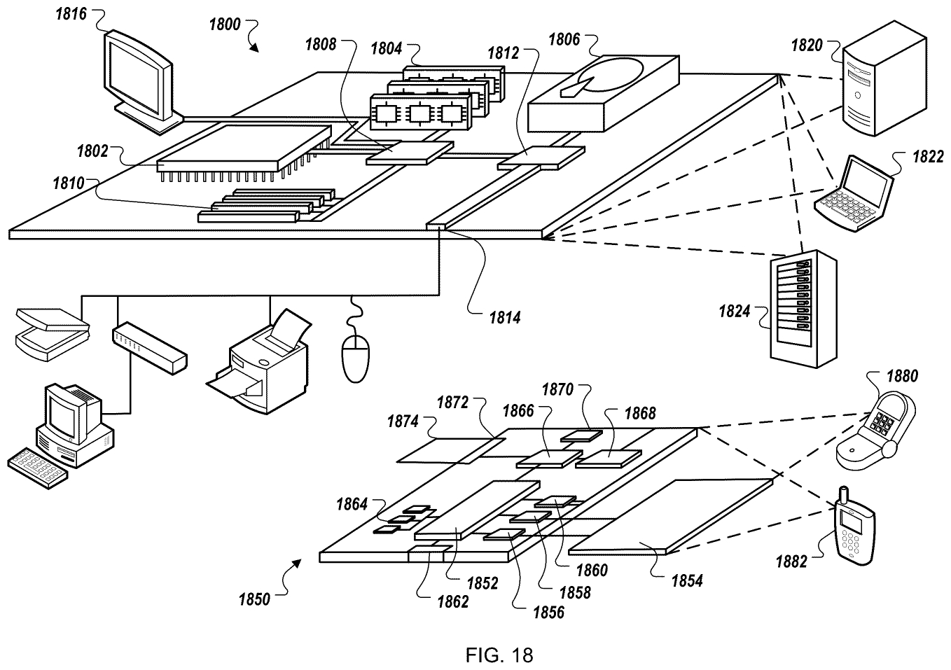

FIG. 16 is a block diagram of an example environment cloud service 1600 used in a data processing system associated with a bed system. Here, the service 1600 is configured to record data related to users' home environment. The service 1600 includes a network interface 1602, a communication manager 1604, server hardware 1606, and server system software 1608. The service 1600 also includes a user identification module 1610, an environmental sensors module 1612, and an environmental factors module 1614. The environmental sensors module 1612 can include a listing and identification of sensors that users identified in the module 1610 to have installed in and/or surrounding their bed (e.g., light, noise/audio, vibration, thermostats, movement/motion sensors). The module 1612 can also store historical readings or reports from the environmental sensors. The module 1612 can be accessed at a later time and used by one or more cloud services described herein to determine sleep quality and/or health information of the users. The environmental factors module 1614 can include reports generated based on data in the module 1612. For example, the module 1614 can generate and retain a report indicating frequency and duration of instances of increased lighting when the user is asleep based on light sensor data that is stored in the environment sensors module 1612.

In the examples discussed here, each cloud service 410 is shown with some of the same components. These same components can be partially or wholly shared between services, or they can be separate. Sometimes, each service can have separate copies of some or all the components that are the same or different in some ways. These components are provided as illustrative examples. In other examples, each cloud service can have different number, types, and styles of components that are technically possible.

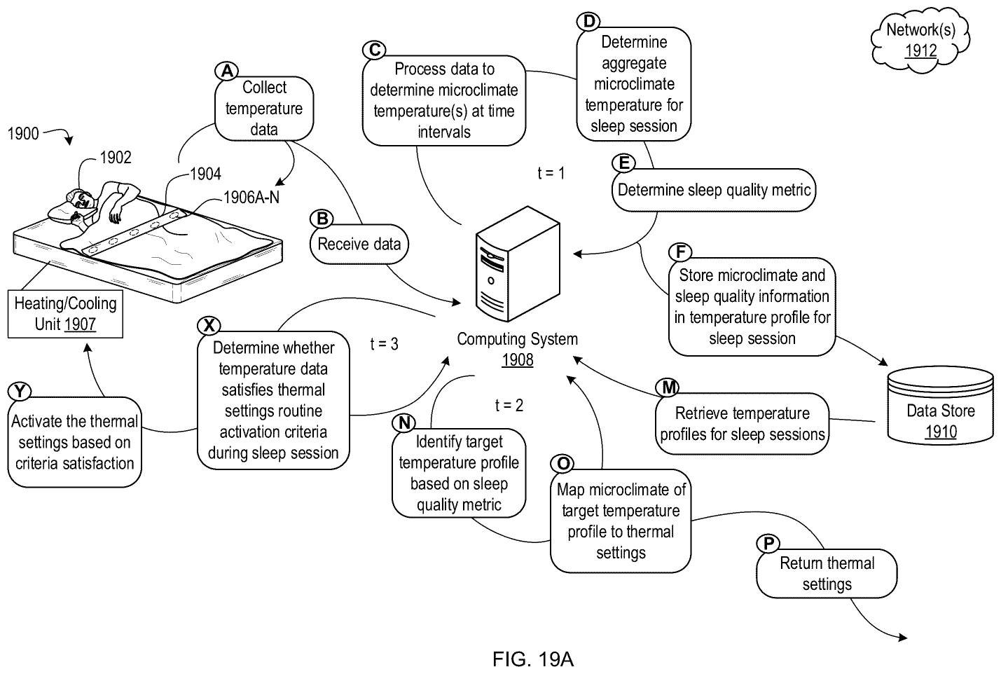

FIG. 17 is a block diagram of an example of using a data processing system associated with a bed to automate peripherals around the bed. Shown here is a behavior analysis module 1700 that runs on the motherboard 402. The behavior analysis module 1700 can be one or more software components stored on the computer memory 512 and executed by the processor 502. In general, the module 1700 can collect data from a variety of sources (e.g., sensors 902, 904, 906, 908, and/or 910, non-sensor local sources 1704, cloud data services 410a and/or 410c) and use a behavioral algorithm 1702 (e.g., machine learning model(s)) to generate actions to be taken (e.g., commands to send to peripheral controllers, data to send to cloud services, such as the bed data cloud 410a and/or the user account cloud 410c). This can be useful, for example, in tracking user behavior and automating devices in communication with the user's bed.

The module 1700 can collect data from any technologically appropriate source (e.g., sensors of the sensor array 406) to gather data about features of a bed, the bed's environment, and/or the bed's users. The data can provide the module 1700 with information about a current state of the bed's environment. For example, the module 1700 can access readings from the pressure sensor 902 to determine air chamber pressure in the bed. From this reading, and potentially other data, user presence can be determined. In another example, the module 1700 can access the light sensor 908 to detect the amount of light in the environment. The module 1700 can also access the temperature sensor 906 to detect a temperature in the environment and/or microclimates in the bed. Using this data, the module 1700 can determine whether temperature adjustments should be made to the environment and/or components of the bed to improve the user's sleep quality and overall comfortability. Similarly, the module 1700 can access data from cloud services to make more accurate determinations of user sleep quality, health information, and/or control the bed and/or peripheral devices. For example, the behavior analysis module 1700 can access the bed cloud service 410a to access historical sensor data 1212 and/or advanced sleep data 1214. The module 1700 can also access a weather reporting service, a 3r d party data provider (e.g., traffic and news data, emergency broadcast data, user travel data), and/or a clock and calendar service. Using data retrieved from the cloud services 410, the module 1700 can accurately determine user sleep quality, health information, and/or control of the bed and/or peripheral devices. Similarly, the module 1700 can access data from non-sensor sources 1704, such as a local clock and calendar service (e.g., a component of the motherboard 402 or of the processor 502). The module 1700 can use this information to determine, for example, times of day that the user is in bed, asleep, waking up, and/or going to bed.

The behavior analysis module 1700 can aggregate and prepare this data for use with one or more behavioral algorithms 1702 (e.g., machine learning models). The behavioral algorithms 1702 can be used to learn a user's behavior and/or to perform some action based on the state of the accessed data and/or the predicted user behavior. For example, the behavior algorithm 1702 can use available data (e.g., pressure sensor, non-sensor data, clock and calendar data) to create a model of when a user goes to bed every night. Later, the same or a different behavioral algorithm 1702 can be used to determine if an increase in air chamber pressure is likely to indicate a user going to bed and, if so, send some data to a third-party cloud service 410 and/or engage a peripheral controller 1002 or 1004, foundation actuators 1006, a temperature controller 1008, and/or an under-bed lighting controller 1010.

Here, the module 1700 and the behavioral algorithm 1702 are shown as components of the motherboard 402. Other configurations are also possible. For example, the same or a similar behavioral analysis module 1700 and/or behavioral algorithm 1702 can be run in one or more cloud services, and resulting output can be sent to the pump motherboard 402, a controller in the controller array 408, or to any other technologically appropriate recipient described throughout this document.

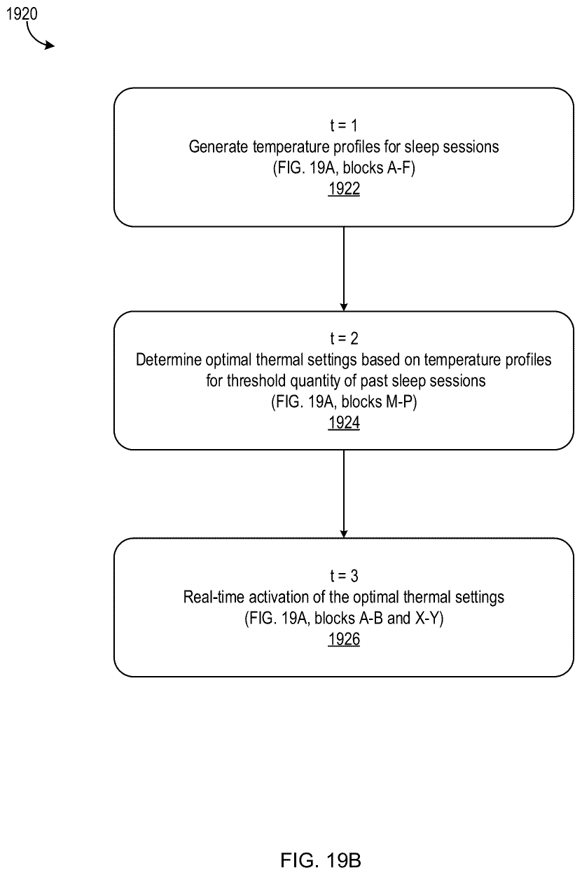

FIG. 18 shows an example of a computing device 1800 and an example of a mobile computing device that can be used to implement the techniques described here. The computing device 1800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1800 includes a processor 1802, a memory 1804, a storage device 1806, a high-speed interface 1808 connecting to the memory 1804 and multiple high-speed expansion ports 1810, and a low-speed interface 1812 connecting to a low-speed expansion port 1814 and the storage device 1806. Each of the processor 1802, the memory 1804, the storage device 1806, the high-speed interface 1808, the high-speed expansion ports 1810, and the low-speed interface 1812, are interconnected using various busses, and can be mounted on a common motherboard or in other manners as appropriate. The processor 1802 can process instructions for execution within the computing device 1800, including instructions stored in the memory 1804 or on the storage device 1806 to display graphical information for a GUI on an external input/output device, such as a display 1816 coupled to the high-speed interface 1808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system). The memory 1804 stores information within the computing device 1800. In some implementations, the memory 1804 is a volatile memory unit or units. In some implementations, the memory 1804 is a non-volatile memory unit or units. The memory 1804 can also be another form of computer-readable medium, such as a magnetic or optical disk. The storage device 1806 is capable of providing mass storage for the computing device 1800. In some implementations, the storage device 1806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1804, the storage device 1806, or memory on the processor 1802.

The high-speed interface 1808 manages bandwidth-intensive operations for the computing device 1800, while the low-speed interface 1812 manages lower bandwidth-intensive operations. Such allocation of functions is exemplary only. In some implementations, the high-speed interface 1808 is coupled to the memory 1804, the display 1816 (e.g., through a graphics processor or accelerator), and to the high-speed expansion ports 1810, which can accept various expansion cards (not shown). In the implementation, the low-speed interface 1812 is coupled to the storage device 1806 and the low-speed expansion port 1814. The low-speed expansion port 1814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter. The computing device 1800 can be implemented in a number of different forms, as shown in the figure. For example, it can be implemented as a standard server 1820, or multiple times in a group of such servers. In addition, it can be implemented in a personal computer such as a laptop computer 1822. It can also be implemented as part of a rack server system 1824. Alternatively, components from the computing device 1800 can be combined with other components in a mobile device (not shown), such as a mobile computing device 1850. Each of such devices can contain one or more of the computing device 1800 and the mobile computing device 1850, and an entire system can be made up of multiple computing devices communicating with each other. The mobile computing device 1850 includes a processor 1852, a memory 1864, an input/output device such as a display 1854, a communication interface 1866, and a transceiver 1868, among other components. The mobile computing device 1850 can also be provided with a storage device, such as a micro-drive or other device, to provide additional storage. Each of the processor 1852, the memory 1864, the display 1854, the communication interface 1866, and the transceiver 1868, are interconnected using various buses, and several of the components can be mounted on a common motherboard or in other manners as appropriate.

The processor 1852 can execute instructions within the mobile computing device 1850, including instructions stored in the memory 1864. The processor 1852 can be implemented as a chipset of chips that include separate and multiple analog and digital processors. The processor 1852 can provide, for example, for coordination of the other components of the mobile computing device 1850, such as control of user interfaces, applications run by the mobile computing device 1850, and wireless communication by the mobile computing device 1850. The processor 1852 can communicate with a user through a control interface 1858 and a display interface 1856 coupled to the display 1854. The display 1854 can be, for example, a TFT (Thin-Film-Transistor Liquid Crystal Display) display or an OLED (Organic Light Emitting Diode) display, or other appropriate display technology. The display interface 1856 can comprise appropriate circuitry for driving the display 1854 to present graphical and other information to a user. The control interface 1858 can receive commands from a user and convert them for submission to the processor 1852. In addition, an external interface 1862 can provide communication with the processor 1852, so as to enable near area communication of the mobile computing device 1850 with other devices. The external interface 1862 can provide, for example, for wired communication in some implementations, or for wireless communication in other implementations, and multiple interfaces can also be used.

The memory 1864 stores information within the mobile computing device 1850. The memory 1864 can be implemented as one or more of a computer-readable medium or media, a volatile memory unit or units, or a non-volatile memory unit or units. An expansion memory 1874 can also be provided and connected to the mobile computing device 1850 through an expansion interface 1872, which can include, for example, a SIMM (Single In Line Memory Module) card interface. The expansion memory 1874 can provide extra storage space for the mobile computing device 1850, or can also store applications or other information for the mobile computing device 1850. Specifically, the expansion memory 1874 can include instructions to carry out or supplement the processes described above, and can include secure information also. Thus, for example, the expansion memory 1874 can be provide as a security module for the mobile computing device 1850, and can be programmed with instructions that permit secure use of the mobile computing device 1850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1864, the expansion memory 1874, or memory on the processor 1852. In some implementations, the computer program product can be received in a propagated signal, for example, over the transceiver 1868 or the external interface 1862.

The mobile computing device 1850 can communicate wirelessly through the communication interface 1866, which can include digital signal processing circuitry where necessary. The communication interface 1866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1868 using a radio-frequency. In addition, short-range communication can occur, such as using a Bluetooth, WiFi, or other such transceiver (not shown). In addition, a GPS (Global Positioning System) receiver module 1870 can provide additional navigation- and location-related wireless data to the mobile computing device 1850, which can be used as appropriate by applications running on the mobile computing device 1850. The mobile computing device 1850 can also communicate audibly using an audio codec 1860, which can receive spoken information from a user and convert it to usable digital information. The audio codec 1860 can likewise generate audible sound for a user, such as through a speaker, e.g., in a handset of the mobile computing device 1850. Such sound can include sound from voice telephone calls, can include recorded sound (e.g., voice messages, music files, etc.) and can also include sound generated by applications operating on the mobile computing device 1850. The mobile computing device 1850 can be implemented in a number of different forms, as shown in the figure. For example, it can be implemented as a cellular telephone 1880. It can also be implemented as part of a smart-phone 1882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input. The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet. The computing system can include clients and servers. A client and server are generally remote from each other and typically interact through a communication network. The relationship of client and server arises by virtue of computer programs running on the respective computers and having a client-server relationship to each other.

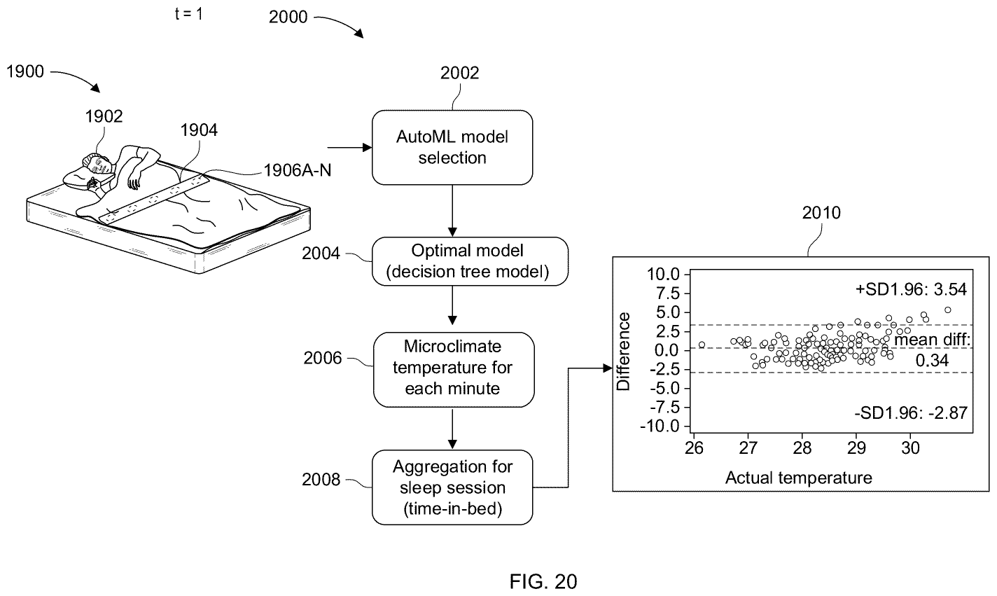

FIG. 19A is a conceptual diagram for determining and applying thermal settings to a bed system 1900 for controlling a microclimate of the bed system 1900 and improving user sleep quality. At least one user 1902 can rest on the bed system 1900. The bed system 1900 can be any of the bed systems described herein. The bed system 1900 can be sized to accommodate two users, such as the user 1902 and a partner. The bed system 1900 may include a sensor strip 1904 having sensors 1906A-N. The bed system 1900 may also include a heating and/or cooling unit 1907.

In brief, the sensor strip 1904 can be attached to a top surface of the bed system 1900. The sensor strip 1904 can extend laterally across the top surface of the bed system 1900, from a left side to a right side of the bed system 1900. In implementations where the bed system 1900 is intended for two users, two sensor strips can be arranged on the top surface of the bed system 1900. A first sensor strip can extend from the left side of the bed system 1900 to a midpoint of the bed system 1900 where a first user rests and a second sensor strip can extend from the right side of the bed system 1900 to the midpoint of the bed system 1900 where a second user rests.

The sensor strip 1904 can include a plurality of the sensors 1906A-N. The sensors 1906A-N can be linearly and uniformly spaced arranged along a length of the sensor strip 1904 and configured to detect sensor data at the top surface of the bed system 1900. The sensors 1906A-N can be equally spaced apart along the length of the sensor strip 1904. For example, for a queen-sized bed system, the sensors 1906A-N can be equally spaced 5.5 inches apart. As another example, for a king-sized bed system, the sensors 1906A-N can be equally spaced 6.5 inches apart.

As described herein, the sensors 1906A-N can be temperature sensors configured to detect microclimate temperature data of the bed system 1900 as the user 1902 rests on top of the bed system 1900. In some implementations, the sensors 1906A-N can be one or more other types of sensors, including but not limited to pressure and/or force sensors. Refer to FIG. 9B for further discussion about the bed system 1900 having the sensor strip 1904 with the sensors 1906A-N.

Components of the bed system 1900, such as the sensors 1906A-N and the heating/cooling unit 1907 can communicate (e.g., wired and/or wireless) with each other and at least a computing system 1908 via network(s) 1912. The computing system 1908 can be configured to perform one or more processes described herein. The computing system 1908 can be part of the bed system 1900, such as a controller of the bed system 1900. The computing system 1908 can also be remote from the bed system 1900, such as a cloud-based computing system or other network of computing devices. The computing system 1908 can also, in some implementations, be an edge computing device. One or more other variations and/or configurations of the computing system 1908 are also possible.

Still referring to FIG. 19A, the processes described herein can be performed at one or more different times. For example, at a first time, t=1, microclimate temperature profiles can be generated for a sleep session of the user 1902 using temperature data collected at the bed system 1900 during the sleep session. Refer to blocks A-F discussed below. The blocks A-F can be performed during each sleep session of the user 1902. Accordingly, the first time t=1 can occur during a current sleep session of the user 1902 and/or some threshold amount of time after the current sleep session of the user 1902 (e.g., once the user 1902 is detected to exit the bed system, once the user 1902 accesses a mobile application about their sleep health/quality via their mobile computing device, after an expected or actual wakeup time/schedule of the user 1902). The first time t=1 can be defined in a variety of other ways. For example, the first time t=1 can quantify any other unit of time.

At a second time, t=2, thermal settings for controlling microclimate conditions of the bed system 1900 during subsequent sleep sessions of the user 1902 can be determined. Refer to blocks M-P discussed further below. The second time can occur during the first time or as part of the first time. Additionally or alternatively, the second time can occur some threshold amount of time after the first time. The second time can also occur at predetermined time intervals. For example, the second time can occur after every 3, 5, 7, 10, 14, etc., consecutive sleep sessions (e.g., where each sleep session is one night's sleep for the user 1902). The second time may also occur whenever any other threshold condition is met (e.g., threshold quantity of consecutive sleep sessions passes, threshold changes in sleep quality occur over some amount of time). In some implementations, the second time can occur before or after every sleep session of the user 1902.

At a third time, t=3, the thermal settings determined at the second time, t=2, can be applied to the bed system 1900. Refer to blocks A-B and X-Y discussed below. The third time can occur during the current sleep session of the user 1902. As a result, the microclimate conditions of the bed system 1900 can be dynamically controlled as the user 1902 rests on the bed system 1900 in order to promote improved sleep quality of the user 1902. In some implementations, the third time can occur before a subsequent sleep session of the user 1902. For example, the third time can be part of or otherwise during the second time, t=2. The computing system 1908 can determine when to implement the thermal settings during the subsequent sleep session based on historic sleep data and/or microclimate temperature data. The computing system 1908 can then load or pre-program the heating/cooling unit 1907 of the bed system 1900 with the determined thermal settings so that during runtime (e.g., during a current sleep session of the user 1902), the thermal settings can be automatically implemented to achieve the optimal microclimate conditions at the bed system 1900.

Referring to the blocks A-F at the first time, t=1, the sensors 1906A-N of the sensor strip 1904 can collect temperature data (block A). The temperature data can be continuously collected throughout a sleep session of the user 1902. The temperature data can be collected at predetermined time intervals during the sleep session of the user 1902 (e.g., epochs, every 30 seconds, every 1 minute, every 3 minutes, every 5 minutes). The temperature data can be transmitted to the computing system 1908 in block B. The temperature data can be transmitted as it is collected, in real-time or near real-time. The temperature data can be transmitted in batch, in some implementations. For example, the temperature data can be transmitted at predetermined time intervals, once a current sleep session of the user 1902 ends, a threshold amount of time after the current sleep session of the user 1902 ends, after a threshold amount of successive/consecutive sleep sessions, and/or before a subsequent sleep session begins. The temperature data may also be transmitted at one or more other points in time before, during, or after the temperature data is collected in block A.

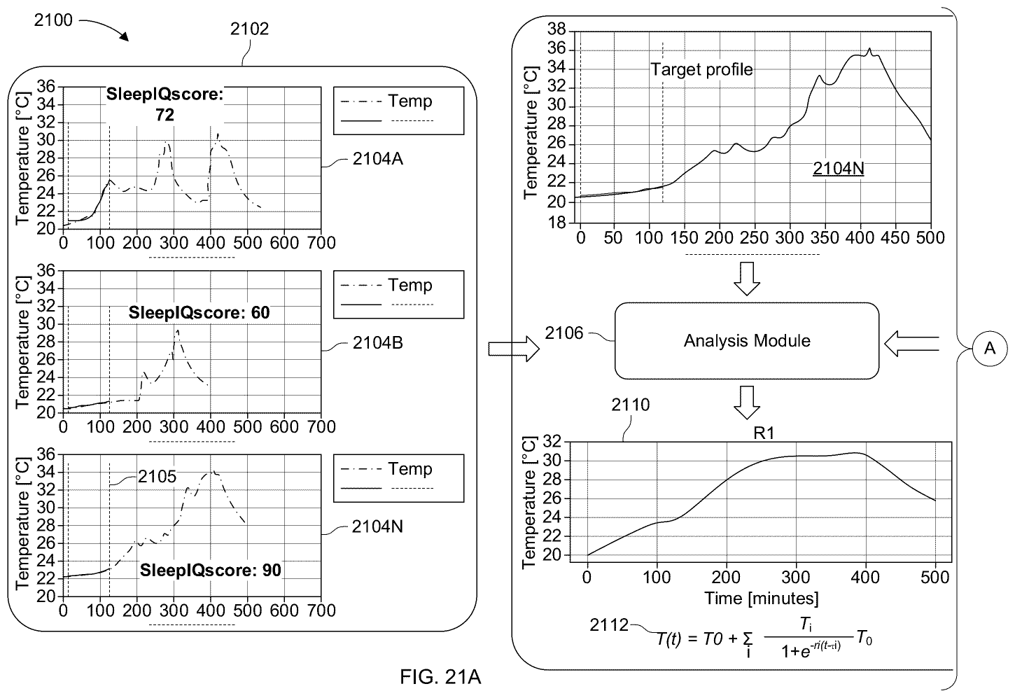

At time t=1, the computing system 1908 can process the received temperature data to determine one or more microclimate temperature values at one or more predetermined time intervals (block C). For example, as shown and described further in reference to the process 2000 in FIG. 20, the computing system 1908 can apply one or more machine learning trained models to the temperature data to determine, based on the data, different microclimate temperature values at the top surface of the bed system 1900 at predetermined time intervals. The predetermined time intervals can vary. For example, the microclimate temperature values can be determined for every 5 minutes, 10 minutes, 15 minutes, 20 minutes, 25 minutes, 30 minutes, etc. of the user 1902's sleep session.

In block D, the computing system 1908 can determine an aggregate microclimate temperature for the entire sleep session of the user 1902. For example, the computing system 1908 can sum all of the microclimate temperature values determined in block C. The computing system 1908 can also average the summed microclimate temperature values to determine the aggregate microclimate temperature value for the entire sleep session of the user 1902. Aggregating many microclimate temperature values in block D can be beneficial to accurately determine an average microclimate temperature for the entire sleep session of the user 1902. One or more other aggregating techniques can be used to determine the aggregate microclimate temperature in block D.

The computing system 1908 can also determine a sleep quality metric for the user 1902 in block E at time t=1. Block E can be performed at the end of the current sleep session. The sleep quality metric can be determine for each sleep session of the user 1902. Block E may also be performed before, during, or after one or more of the blocks A-D. The sleep quality metric can be a numeric value indicating a level of sleep quality experienced by the user 1902 during the sleep session. The sleep quality metric can indicate how well the user 1902 slept, which can be based on a variety of inputs and/or sensor data collected by sensors and other components of the bed system 1900. For example, as described above, the sleep quality metric can be determined based on pressure data sensed at the bed system 1900 during the sleep session, where changes in the pressure can indicate movement of the user 1902 during their sleep (e.g., restlessness), snoring, changes in heartrate, changes in respiration rate, etc. Machine learning techniques can be used to model the pressure data to a value or values indicating the sleep quality metric for the user 1902.

The computing system 1902 can store the microclimate and sleep quality information in a temperature profile for the sleep session of the user 1902 (block F). This information can be stored in a data store 1910. The data store 1910 can be any type of database, data storage, and/or cloud-based storage. The data store 1910 can be part of the computing system 1908, in some implementations. The computing system 1902 can store the aggregate microclimate temperature and/or the microclimate temperature values for the time intervals throughout the sleep session in the temperature profile for the sleep session. The computing system 1902 can store this temperature information once it is determined in either of blocks C or D. The computing system 1902 can also store the sleep quality metric in association with information about the sleep session when the sleep quality metric is determined in block E. The microclimate and/or sleep quality information can then be retrieved from the data store 1910 at a later time (e.g., during the second and/or third times) for use in additional processing.

Referring to the blocks M-P at the second time, t=2, the computing system 1902 can retrieve temperature profiles for one or more past sleep sessions of the user 1902 (block M). The temperature profiles can be retrieved from the data store 1910. The computing system 1902 can retrieve the temperature profiles for a threshold quantity of the past sleep sessions. For example, the computing system 1902 can retrieve the temperature profiles for a past 3, 5, 7, 8, 9, etc. past sleep sessions of the user 1902. The computing system 1902 can retrieve the temperature profiles for successive sleep sessions. The computing system 1902 can also retrieve the temperature profiles for sleep sessions that may not necessarily be successive (e.g., a first sleep session, a third sleep session, a fifth sleep session). The computing system 1902 can retrieve the temperature profiles for any quantity of past sleep sessions that can be used to accurately and efficiently determine thermal settings that are intended to control the microclimate at the bed system 1900 during subsequent sleep sessions of the user 1902, and therefore result in the user 1902 experiencing high levels of sleep quality. In some implementations, the threshold quantity of past sleep sessions can vary based on season (e.g., winter, summer, spring, fall), circadian rhythm of the user 1902, month, year, age, and/or other demographic information about the user 1902.

In yet some implementations, the computing system 1908 can retrieve temperature profiles for a threshold quantity of past sleep sessions of a general population of users. The population of users can include the user 1902. The population of users can also include users that have similar health data, sleep quality data, geographic location, and/or other demographics as the user 1902. Using the temperature profiles from sleep sessions of a robust set of users can provide for accurate identification of a target temperature profile that may result in improved sleep quality for the particular user 1902.

The computing system 1908 can identify a target temperature profile amongst the retrieved temperature profiles in block N. The computing system 1908 can identify the target temperature profile based on assessing the sleep quality metric associated with each of the retrieved temperature profiles. The target temperature profile can be identified as having a highest sleep quality metric amongst all of the retrieved temperature profiles. As another example, the target temperature profile can be identified as having a sleep quality metric that exceeds a threshold sleep quality value. The threshold sleep quality value can be, in some examples, a numeric value of 90. One or more other criteria may also be used to identify the target temperature profile in block N.

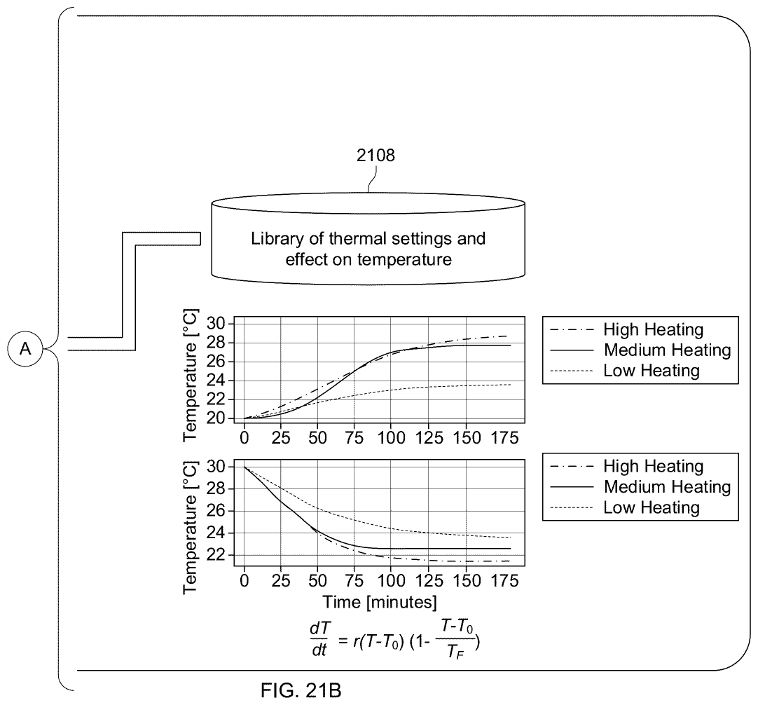

Next, in block O, the computing system 1908 can map the microclimate information of the target temperature profile to thermal settings. As described in reference to the process 2100 in FIGS. 21A-B, the computing system 1908 can use machine learning models and techniques to decompose and map the microclimate temperature values in the target temperature profile to predetermined thermal routine settings. Therefore, the computing system 1908 can determine and/or identify thermal routine settings that, when implemented at the bed system 1900, can replicate the microclimate temperature values (or some of the microclimate temperature values) in the target temperature profile when the user 1902 experienced the threshold level of sleep quality.

The computing system 1908 can return the thermal settings in block P. For example, the computing system 1908 can store the thermal settings in the data store 1910. The computing system 1908 can additionally or alternatively store the thermal settings in local memory so that the thermal settings can be quickly retrieved and executed at runtime during the subsequent sleep sessions of the user 1902. As another example, the computing system 1908 can transmit the thermal settings to the heating/cooling unit 1907 of the bed system 1900 or a controller of the bed system 1900 such that the heating/cooling unit 1907 can be preprogrammed with the thermal settings. Therefore, at runtime during the user 1902's sleep session(s), the heating/cooling unit 1907 can automatically execute the thermal settings, as described below.

Referring to the blocks A-B and X-Y at the third time, t=3, the sensors 1906A-N can collect temperature data during the user 1902's sleep session, such as their subsequent sleep session(s) (block A). The collected temperature data can then be transmitted to and received by the computing system 1908 (block B). The computing system 1908 can determine whether the received temperature data satisfies thermal-settings-routine activation criteria during the current sleep session of the user 1902 (block X). The criteria can be used to determine whether and/or when to activate thermal settings at the bed system 1900. For example, the computing system 1908 can determine whether a current temperature at the top surface of the bed system 1900 has dropped below a threshold temperature, where that threshold temperature corresponds to a threshold level of preferred sleep quality (e.g., the bed is too cold for the user 1902 to comfortably fall asleep or stay asleep). If the current temperature is less than the threshold temperature, then the criteria may be satisfied and the computing system 1908 can transmit instructions to the heating/cooling unit 1907 to activate the thermal settings (block Y). As another example, if the current temperature exceeds the threshold temperature (e.g., the bed is too warm for the user 1902 to comfortably fall asleep or stay asleep), then the criteria may be satisfied and the thermal settings can be activated in block Y. As another example, the computing system 1908 can compare the temperature data to one or more threshold temperature values and/or temperature value ranges. The computing system 1908 can also compare derivatives of the received temperature data to threshold values and/or ranges to determine whether to activate the thermal settings in block Y.

One or more other criteria can be used at runtime during the user 1902's sleep session. The criteria can also establish different rules and/or thresholds based on the user 1902's age, health conditions, gender, geographic location, seasons, etc. The criteria can also establish different rules and/or threshold for different intervals and/or sleep stages during the user 1902's sleep session. Therefore, different thermal settings can be activated at different points during the user 1902's sleep session based on whether the current temperature of the bed system 1900 should be decreased, increased, and/or maintained in order to achieve the desired microclimate for improving/maintaining the user 1902's sleep quality. In some implementations, the heating/cooling unit 1907 and/or a controller of the bed system 1900 can make the determination in block X and activate the thermal settings in block Y.

In some implementations, the user 1902 can override the thermal settings and/or selectively determine when the thermal settings are activated at the bed system 1900. For example, the thermal settings can be presented as a suggestion in a GUI display at a computing device of the user 1902. The user 1902 can then choose whether they would like to activate the thermal settings at a present time, during a next/subsequent sleep session, or at any other time. The user 1902 may also choose an option to permit the heating/cooling unit 1907 to automatically activate the thermal settings when, for example, it is determined that the routine activation criteria is satisfied. As another example, the user 1902 may choose to ignore the suggested thermal settings. As a result, the thermal settings may not be activated at the bed system 1900 during the subsequent sleep session(s) of the user 1902.

In some implementations, blocks A-B and/or X-Y can be performed at various windows of time during the user 1902's current sleep session. For example, the blocks X-Y can be performed every 5 minutes, 10 minutes, 15 minutes, 20 minutes, 30 minutes, 1 hour, 2 hours, 3 hours, etc., during the user 1902's sleep session. As another example, the blocks X-Y can be performed when certain sleep stages or physical movement of the user 1902 are determined and/or detected using the disclosed techniques. In some implementations, the blocks X-Y can be performed continuously, throughout the entire sleep session of the user 1902.

Furthermore, as described herein, one or more of the blocks A-F, M-P, and X-Y can be performed on the edge, for example at a controller of the bed system 1900, the computing device of the user 1902, and/or an edge computing device. For example, blocks X-Y can be performed on the edge in order to allow for quick, runtime adjustments to be made to the microclimate of the bed system 1900 during the user 1902's sleep session. As another example, blocks A-F and/or M-P can be performed remotely from the bed system 1900. Performing the blocks A-F and/or M-P remotely can take advantage of greater offline processing power at the computing system 1908. Robust and accurate determinations can be made by leveraging the remote, offline processing power of the computing system 1908.

In some implementations, the bed system 1900 can be occupied by two users. Temperature profiles can be modeled/generated in blocks A-F for each side of the bed system 1900 (e.g., for each user). Target temperature profiles can also be determined in blocks M-P for each side of the bed system 1900. The target temperature profile for a first side of the bed system 1900 can also be determined using criteria that takes into account effects of microclimate temperature values at a second side of the bed system 1900. Similarly, the target temperature profile for the second side of the bed system 1900 can be determined using criteria that takes into account effects of microclimate temperature values at the first side of the bed system 1900. After all, sometimes when a heating or cooling routine is activated at one of the sides of the bed system 1900, heated or cooled air from that side of the bed system 1900 can permeate to the other side of the bed system 1900, thereby effecting the microclimate of the other side of the bed system 1900. Furthermore, whether and which thermal settings to activate in blocks X-Y can be determined for each of the first and second sides of the bed system 1900 while the users are in the bed system 1900. Sometimes, the blocks X-Y can be performed on the edge (e.g., by the heating/cooling unit 1907 and/or a controller of the bed system 1900) to dynamically adjust a microclimate at each side of the bed system 1900.

FIG. 19B is a flowchart of a process 1920 for controlling the microclimate of the bed system as shown in FIG. 19A. The process 1920 can be performed by the computing system 1908 described in FIG. 19A. One or more blocks in the process 1920 can also be performed by one or more other components described herein.

Referring to the process 1920 in FIG. 19B, temperature profiles for sleep sessions of a user of the bed system can be determined at time t=1 (block 1922). Refer to blocks A-F in FIG. 19A for further discussion.

Optimal thermal settings can be determined based on the temperature profiles for a threshold quantity of past sleep sessions in block 1924, at time t=2. Refer to blocks M-P in FIG. 19A for further discussion.

The optimal thermal settings can then be activated in real-time, during subsequent sleep sessions of the user at time t=3 (block 1926). Refer to blocks A-B and X-Y in FIG. 19A for further discussion.

FIG. 20 is a conceptual diagram of a process 2000 for determining microclimate temperature data of the bed system 1900 when the user 1902 rests on the bed system 1900. As described herein, the process 2000 can leverage temperature data collected by the sensors 1906A-N (e.g., 5 temperature sensors) of the sensor strip 1904 throughout a sleep session of the user 1902 to approximate or otherwise determine microclimate temperature values of the bed system 1900 during the sleep session of the user 1902. The sensors 1906A-N can collect the temperature data at various sampling rates. For example, the temperature data can be sampled at approximately 0.2 Hz per signal.

The process 2000 can be performed by the computing system 1908 described in FIG. 19A. Moreover, the process 2000 is similar to or the same as blocks A-F described in FIG. 19A. The process 2000 can be performed at time t=1, during each sleep session of the user 1902. Therefore, the process 2000 can be performed to generate temperature profiles for each sleep session of the user 1902, which can then be used to determine thermal settings for subsequent sleep sessions that may control the microclimate of the bed system 1900, thereby causing the user 1902 to experience improved sleep quality during the subsequent sleep sessions.

As shown in the process 2000 in FIG. 20, the temperature data can be collected and processed using an autoML model selection (block 2002). The autoML model selection can be used to test one or more model candidates and select one of the model candidates for runtime use. The model that is selected can provide preferred or at least threshold results/outcomes from running the model using the collected data (e.g., testing of the models can be performed based on analyzing accuracy of the models using validation data). Here, the model candidates can include, but are not limited to, random forest models, support vector machines (SVMs), lineage regression models, and/or decision trees (boosted or regular decision trees). In the example of the process 2000, a boosted decision tree model was selected.

Next, the selected model can be applied to the temperature data in block 2004. As mentioned above, here the boosted decision tree model was selected. Parameters for the model can be determined using machine learning techniques. The model can be trained using data collected during prior sleep sessions of the user 1902 and/or a general population of users. The training data can include temperature data collected by temperature sensors at the bed system 1900 of the user 1902 and/or bed systems of the users in the general population of users. In some implementations, the training data can include temperature data that was collected as ground-truth data.

By applying the optimal model to the collected temperature data, microclimate temperature values for each minute, or other predetermined intervals of time, can be determined (block 2006).

Then, the microclimate temperature values for each minute can be aggregated to determine an average microclimate temperature for the sleep session of the user 1902 (block 2008). The sleep session can be a time-in-bed of the user 1902. For example, as described above, presence of the user 1902 can be detected using one or more sensors (e.g., pressure sensors, force sensors, temperature sensors, any combination thereof) of the bed system 1900. Using the presence detections, a computing system as described herein can determine when the user 1902 is in the bed system 1900, or otherwise the time-in-bed of the user 1902. The computing system can then determine the microclimate temperature values for each minute of the time-in-bed of the user 1902 as well as the average microclimate temperature for the entire time-in-bed of the user 1902.

The average microclimate temperature for the sleep session, and/or the microclimate temperature values per minute, can be returned, as shown in graph 2010. The graph 2010 is a Bland-Altman graph, which can be used to quantify an accuracy of estimation of the temperature as well as to evaluate whether the accuracy depends on a value of the temperature to be replicated. A horizontal axis of the graph 2010 indicates actual temperature. The actual temperature values are those that can be replicated using the disclosed technology. A vertical axis of the graph 2010 indicates a difference between the actual temperature values and estimated temperature values that are determined using the disclosed technology. Dashed horizontal lines in the graph 2010 indicate limits of agreement that characterize intervals within which a threshold amount (e.g., 95%) of the estimated temperature values deviate from the actual temperature values. To obtain the threshold amount (e.g., 95% confidence interval), a standard deviation of the difference between actual and estimated temperature values can be determined, multiplied by a threshold factor (e.g., a factor of 1.96), and then added or subtracted from a mean temperature value to determine upper and lower limits of agreement, respectively.

FIGS. 21A-B is a conceptual diagram of a process 2100 for determining thermal settings to implement during subsequent sleep sessions of a user based on processing data that corresponds to past sleep sessions of the user. The process 2100 corresponds to time t=2 as described in reference to blocks M-P in FIG. 19A. The process 2100 can be performed by the computing system 1908 described above. One or more blocks in the process 2100 can also be performed by other components and/or computing systems described throughout this disclosure.

Referring to the process 2100, a target temperature profile can be identified in block 2102. As described previously, the temperature profiles can be generated for each sleep session of the user. The temperature profiles can indicate microclimate temperature values at various time intervals during each past sleep session. Each temperature profile can be represented as a graph depicting the microclimate temperature values throughout the sleep session of the user. In the illustrative example of FIGS. 21A-B, graphs 2104A, 2104B, and 2104N indicate temperature profiles for three different sleep sessions of the user. Each temperature profile can also be associated with a sleep quality metric, as determined using the disclosed techniques. Without loss of generality, the sleep quality metric can be a sleep quality score or other quantifier of sleep quality, including but not limited to subjective sleep quality metrics (e.g., user-perceived sleep quality).

In the example of FIGS. 21A-B, the sleep session shown in the graph 2104A has a sleep quality metric of 72, the sleep session shown in the graph 2104B has a sleep quality metric of 60, and the sleep session shown in the graph 2104N has a sleep quality metric of 90. Although three graphs 2104A, 2104B, and 2104N are shown for three past sleep sessions of the user, additional or fewer temperature profiles can be retrieved in block 2102. Moreover, as shown in the graph 2104N, a vertical line 2105 indicates a time at which the user falls asleep. Once the user falls asleep, the microclimate temperature values detected at the top surface of the bed system may increase and decrease, as shown in the graphs 2104A, 2104B, and 2104N.

In block 2102, a target temperature profile can be identified. The target temperature profile can be identified by ranking the temperature profiles for the three sleep sessions that were retrieved. The temperature profiles can be ranked based on their respective sleep quality metrics, from highest to lowest sleep quality metric. In the example of FIGS. 21A-B, the target temperature profile can be identified as the graph 2104N, which has the highest sleep quality metric value of 90 amongst the temperature profiles.

The target temperature profile, as shown by the graph 2104N, can then be decomposed into a summation of thermal settings in block 2106. An analysis module of the computing system described herein can be configured to decompose the microclimate temperature values of the target temperature profile into thermal settings. In other words, the analysis module can identify relationships between and correlate the microclimate temperature values with thermal settings. The thermal settings in the example of FIGS. 21A-B are shown in graph 2110. The target temperature profile can be decomposed using equation 2112 and various parameters determined for each thermal setting. Refer to the table below for further discussion about the parameters for each settings.

TABLE 1
estimated parameters for thermal settings

| | T₀[° C.] | T_{F}[° C.] | T₀ + T_{F}[° C.] | r[1/min] | t[min] | |
|---|---|---|---|---|---|---|
| Low heating | 21.9 | 3.38 | 25.3 | 0.03 | 37.1 | 1.7° C. increase |
| (H1) | | | | | | in 37 minutes |
| Mid heating | 21.0 | 7.84 | 28.8 | 0.06 | 63.5 | 3.9° C. increase |
| (H2) | | | | | | in 64 minutes |
| High heating | 21.0 | 10.9 | 31.9 | 0.03 | 55.2 | 5.5° C. increase |
| (H3) | | | | | | in 55 minutes |
| Low cooling | 29.0 | −5.5 | 22.0 | 0.03 | 30.0 | 2° C. increase |
| (C1) | | | | | | in 30 minutes |
| Mid cooling | 29.0 | −8.27 | 20.7 | 0.06 | 27.3 | 4.1° C. increase |
| (C2) | | | | | | in 27 minutes |
| High cooling | 24.0 | −4.84 | 19.1 | 0.05 | 29.9 | 2.4° C. increase |
| (C3) | | | | | | in 30 minutes |
| Turn OFF | 32.1 | −9.8 | 22.3 | 0.02 | 34.4 | 4.9° C. increase |
| engine | | | | | | in 34 minutes |

The computing system can access a library 2108 of thermal settings and effects the thermal settings have on microclimate temperature of the bed system as part of block 2106. The computing system can use the retrieved information to determine thermal settings that can be used to replicate the target temperature profile during subsequent sleep sessions of the user. For example, the computing system can map the microclimate temperature values to thermal settings using the retrieved information. Sometimes, the computing system may use piecewise logistic fitting with non-overlapping windows that last a threshold amount of time. As described herein, the piecewise logistic fitting can include segmenting the target temperature profile into non-overlapping temporal windows. Without loss of generality, the windows can be 60 minutes long. Sometimes, one or more of the windows can be longer than 60 minutes. For example, a last window can be longer than 60 minutes. For each window, the computing system can implement an algorithm that fits a logistic type of function, such as: T(t)=T0+a*(1+exp(−bt+c))\hat{ }−1, where T is temperature and t is time. The threshold amount of time can, in some implementations, be 60 minutes long. The threshold amount of time can be longer or shorter periods of time. The computing system can implement one or more rules, algorithms, and/or machine learning techniques (e.g., machine learning trained models) in block 2106 in order to determine the thermal settings to be implemented during subsequent sleep sessions of the user, as described further below.

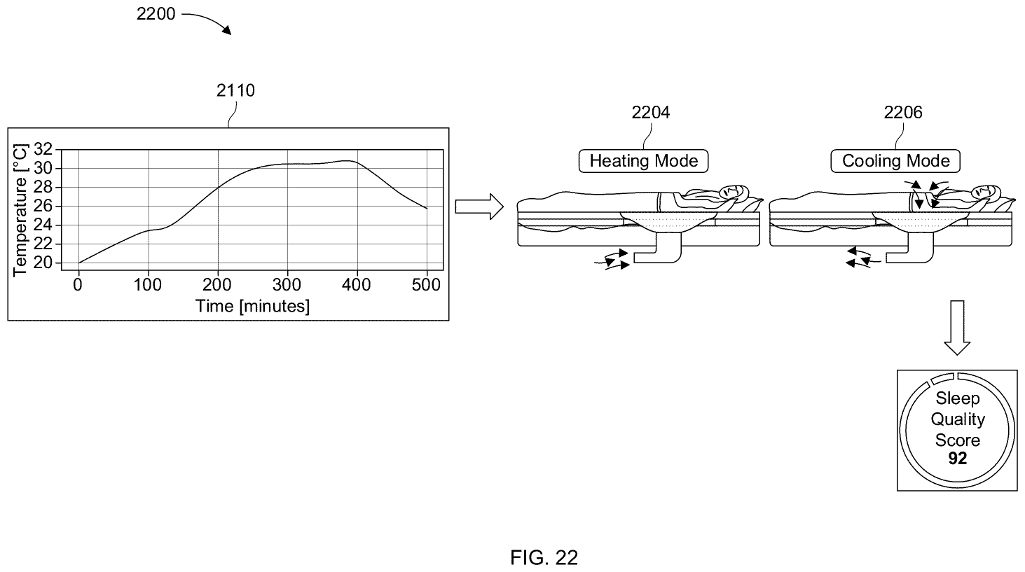

FIG. 22 is a conceptual diagram of a process 2200 for applying the thermal settings determined in the process of FIGS. 21A-B to improve the user's level of sleep quality during subsequent sleep sessions. The process 2200 can be similar to or the same as blocks X-Y at time t=3 in FIG. 19A.

The process 2200 can be performed by the computing system 1908 described above. The computing system 1908 can have one or more processors that receive and execute instructions causing the computing system 1908 to perform one or more blocks of the process 2200. The computing system 1908 can also include a network or communication interface that provides for the computing system 1908 to communicate with other components, such as the data store 1910 and/or the heating/cooling unit 1907 of the bed system 1900 (refer to FIG. 19A). One or more blocks of the process 2200 may also be performed by other components described herein.

Referring to the process 2200, the determined thermal settings 2110 from FIGS. 21A-B can be applied to the bed system. More particularly, the computing system 1908 can execute instructions to pre-program the heating/cooling unit 1907 of the bed system 1900 with the thermal settings 2110 so that during subsequent sleep sessions of the user, the thermal settings 2110 can be automatically activated. The thermal settings 2110 can be retrieved, by the computing system 1908, from the data store 1910. In some implementations, the thermal settings 2110 can be stored in local memory or a data store of the computing system 1908 for quicker access and retrieval in the process 2200. The thermal settings 2110 are expected to result in the user experiencing high sleep quality close to that of the target temperature profile identified in the process 2100 of FIGS. 21A-B. In some implementations, several thermal settings can be stored and/or programmed into the heating/cooling unit 1907, where each of the thermal settings can be triggered/applied based on different external factors. For example, different thermal settings may be activated in one season versus another (e.g., winter versus summer) to promote quality sleep. Different thermal settings may also be activated depending on an age of the user and/or a day of the week. One or more other factors may impact what thermal settings are applied to the bed system 1900 in the process 2200.

The thermal settings 2110 can be activated in a heating mode 2204. Additionally or alternatively, the thermal settings 2110 can be activated in a cooling mode 2206. As described in reference to FIG. 19A, the temperature sensors 1906A-N can detect current temperature values at a top surface of the bed system 1900. The temperature values can be transmitted to a controller of the bed system 1900 and/or the computing system 1908 for processing and analysis. The computing system 1908 can, for example, determine whether the temperature values satisfy criteria to activate the heating mode 2204, the cooling mode 2206, or no activation of the heating/cooling unit 1907.

For example, if the current temperature values of the bed system 1900 are greater than some threshold temperature value or range (where the threshold value or range indicates a preferred or optimal temperature for the user to achieve improved sleep quality), the computing system 1908 can transmit instructions to the heating/cooling unit 1907 of the bed system 1900 that cause the heating/cooling unit 1907 to activate the thermal settings 2110 in the cooling mode 2206. Sometimes, the heating/cooling unit 1907 can determine whether the current temperature values satisfy the criteria to activate the thermal settings 2110 in the cooling mode 2206, instead of the computing system 1908. In the cooling mode 2206, the heating/cooling unit 1907 of the bed system 1900 can be configured to exhaust warm air from the bed system 1900. A reversible fan may also be used to pull air and/or heat from the microclimate at the top surface of the bed system. The cooling mode 2206 may be activated, by the heating/cooling unit 1907, for a duration of time needed to lower the microclimate at the top surface of the bed system 1900 to the threshold temperature value or range. The cooling mode 2206 can remain activated until the threshold temperature value or range is detected by the temperature sensors of the bed system 1900. The heating/cooling unit 1907 can monitor current temperature values sensed by the temperature sensors 1906A-N to determine when to deactivate or turn off the cooling mode 2206. The computing system 1908 can additionally or alternatively monitor the current temperature values to determine when to deactivate or turn off the cooling mode 2206.

As another example, if the current temperature of the bed system 1900 is less than the threshold temperature value or range, the computing system 1908 can transmit instructions to the heating/cooling unit 1907 of the bed system 1900 that cause the heating/cooling unit 1907 to activate the thermal settings 2110 in the heating mode 2206. In the heating mode 2204, surrounding/ambient air can be drawn into the bed system by components of the heating/cooling unit 1907. A heater of the heating/cooling unit 1907 can also be activated with closed-loop monitoring performed by the computing system 1908 of the microclimate at the top surface of the bed system 1900 (in other words, the computing system 1908 can continuously receive temperature values from the temperature sensors 1906A-N while the heater is activated and determine when to deactivate or turn off the heater so that the heater does not cause the microclimate of the bed system 1900 to exceed some threshold temperature value). The reversible fan of the heating/cooling unit 1907 may also be used to push heated air into the microclimate at the top surface of the bed system 1900. The heating mode 2204 may be activated for a duration of time needed to raise the microclimate at the top surface of the bed system 1900 to the threshold temperature value or range. The heating mode 2204 can remain activated until the threshold temperature value or range is detected by the temperature sensors 1906A-N of the bed system 1900. As described above, the computing system 1908 can continuously monitor the temperature values detected by the temperature sensors 1906A-N to determine when to deactivate or turn off the heating mode 2204. The computing system 1908 can then transmit instructions to the heating/cooling unit 1907 that, when executed, cause the heating/cooling unit 1907 to deactivate the heating mode 2204. Sometimes, the heating/cooling unit 1907 or another component of the bed system 1900 (e.g., a controller) can monitor the temperature values and determine when to deactivate the heating mode 2204.

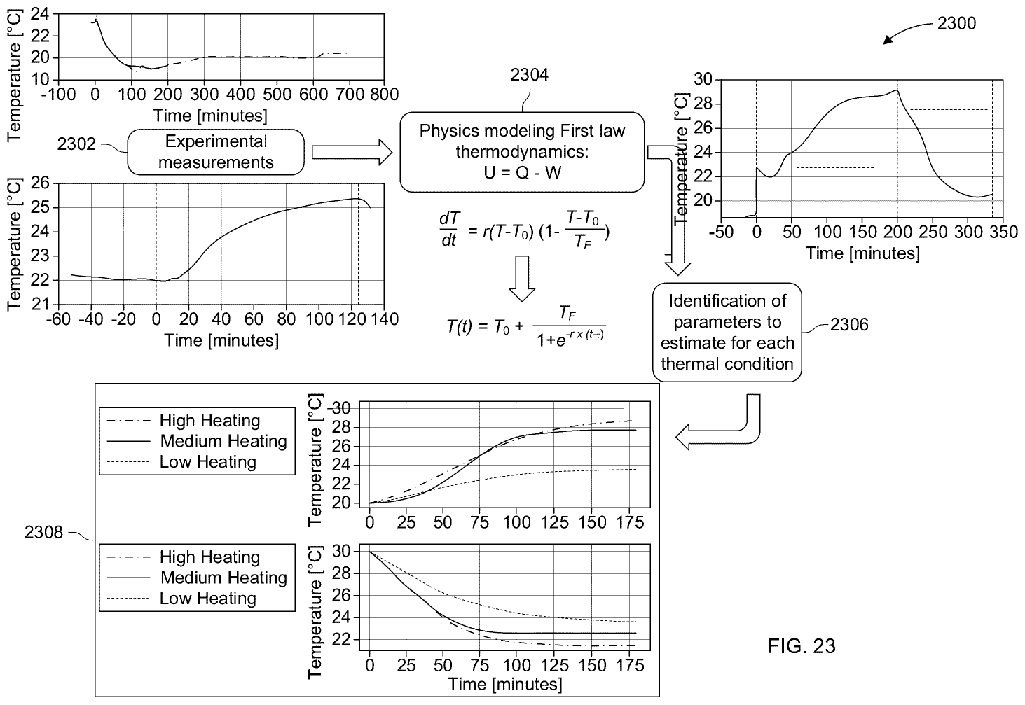

FIG. 23 is a conceptual diagram of a process 2300 to model microclimate temperature data collected at a bed system to thermal settings. The process 2300 is the same as or similar to blocks A-F that are performed at time t=1 in FIG. 19A. The process 2300 can be performed by the computing system 1908, as described herein. One or more blocks in the process 2300 can also be performed by other components described throughout this disclosure.

As shown in the process 2300, temperature data can be collected during sleep sessions of a user in block 2302. Physics modeling techniques can be used in block 2304 to measure changes in the collected temperature data that can be correlated with one or more thermal settings (e.g., cooling down or warming up the bed system). Physics modeling and experimental validation techniques can be used. The type of thermal regulation described throughout this disclosure provides for a logistic change model where an instantaneous rate of temperature change can be proportional to an average rate of change “r>0”, a current temperature “T” of the bed system, and a difference between the current temperature and a final temperature “T₀+T_{F}” of the bed system. See Equations 1 and 2 below.

$$\frac{dT}{dt} = r ( T - {T}_{0} ) ( 1 - \frac{T - {T}_{0}}{{T}_{F}} ) \qquad Equation 1$$

$$T ( t ) = {T}_{0} + \frac{{T}_{F}}{1 + {e}^{- r \times ( t - τ )}} \qquad Equation 2$$

When a heating thermal setting is applied to a bed system, a microclimate temperature of the bed system increases according to the first law of thermodynamics (internal energy U=heat added Q−Work W), which results in a logistic growing type of equation (see Error! Reference source not found.), where “r” is a mean increase in temperature by unit of time, T₀ is initial temperature, and T₀+T_{F} is a maximum temperature the microclimate can attain. A solution of the differential equation leads to a logistic growing curve, as shown by equation 2, which characterizes Temperature as a function of time where “τ” is a half-time parameter that indicates a half-temperature-increase time (e.g., when t=τ, then T=T₀+0.5T_{F}).

Based on the processing in block 2304, a model can be trained. The model can be used to identify parameters for different scenarios (e.g., when the bed system microclimate increases beyond a threshold temperature value or range, when the bed system microclimate decreases below the threshold temperature value or range) in which thermal settings may be activated (block 2306). The parameters can, for example, be identified for thermal conditions that may include but are not limited to high heating, medium heating, low heating, high cooling, medium cooling, low cooling, and off. Using the model and identified parameters, thermal settings 2308 can be returned to be used in response to the various thermal conditions that may be detected/determined at the bed system. The parameters T_{F}, r, and τ for each thermal setting can be estimated empirically by fitting the model to the measured temperature values resulting from each of the thermal settings when activated at the bed system.

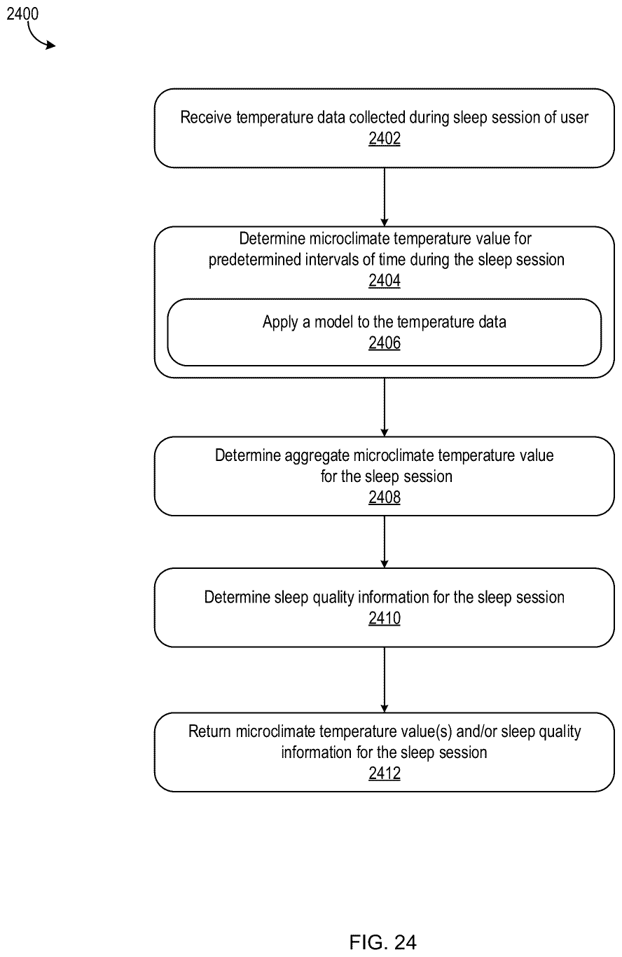

FIG. 24 is a flowchart of a process 2400 to determine microclimate temperature data of a bed system when a user rests on the bed system. The process 2400 is similar to or the same as blocks A-F in FIG. 1A and the process 2000 in FIG. 20. The process 2400 can be performed during each sleep session of the user. In some implementations, the process 2400 may only be performed during some sleep sessions (e.g., every other sleep session, over 3 consecutive sleep sessions, etc.). Moreover, the process 2400 can be performed not only to determine microclimate temperature data over a particular sleep session but also to determine whether to activate thermal settings at the bed system to improve the user's level of sleep quality.

The process 2400 can be performed by the computing system 1908 described in reference to FIG. 19A. The process 2400 can also be performed by one or more other computing systems and/or devices described herein, including but not limited to a controller of the bed system, user computing devices, edge computing devices, remote computing devices, and/or cloud-based systems. For illustrative purposes, the process 2400 is described from the perspective of a computing system.

Referring to the process 2400 in FIG. 24, the computing system can receive temperature data collected during a sleep session of a user at a bed system (block 2402). The temperature data can be received from at least one temperature sensor of the bed system. As described herein, the at least one temperature sensor can be configured to a sensor strip. The sensor strip can be removably attached to a top surface of the bed system on which the user rests. The at least one temperature sensor can, in some implementations, include 5 temperature sensors linearly arranged along the sensor strip. The 5 temperature sensors can be equally spaced apart by a threshold distance along the sensor strip.

The computing system can determine microclimate temperature values for predetermined intervals of time during the sleep session in block 2404. For example, the computing system can apply one or more models to the temperature data (block 2406). The model(s) can be trained to approximate, based on the temperature data, a microclimate temperature value for each of the predetermined intervals of time during the sleep session. The predetermined intervals of time can be 1-minute segments during/throughout the sleep session. 1-minute segments can be used due to relatively slow variation of the microclimate temperature throughout the sleep session. One or more other predetermined intervals of time are also possible. For example, the predetermined intervals of time may include, but are not limited to, 30-second segments, 1.5-minute segments, 2-minute segments, 3-minute segments, 5-minute segments, etc. Model training may also include using autoML model selection described in reference to the process 2000 in FIG. 20 so that a model that satisfies threshold selection criteria can be selected and deployed during runtime use.

The computing system can determine an aggregate microclimate temperature value for the sleep session in block 2408. The aggregate microclimate temperature value can be determined based on averaging, or otherwise aggregating, the microclimate temperature values for the predetermined intervals of time during the sleep session. Sometimes, block 2408 may not be performed. Aggregating the microclimate temperature values can advantageously provide an aggregate overall microclimate temperature value for the entire sleep session of the user. The aggregate microclimate temperature value can beneficially be used as a summary metric for microclimate temperatures throughout the sleep session. The aggregate microclimate temperature value can also beneficially be used when a thermal system is configured in a thermostat module of the bed system and the temperature is maintained based on that aggregate value.

The computing system can determine sleep quality information for the sleep session of the user in block 2410. The sleep quality information can include a numeric value indicating a level of sleep quality experienced by the user during the sleep session, as described throughout this disclosure. The sleep quality information can indicate the quality of sleep experienced during predetermined periods of time during the sleep session (e.g., at each sleep stage, in 5 minute windows, 10 minute windows, 20 minute windows, 30 minute windows). The sleep quality information can also indicate the quality of sleep experienced over the entire sleep session. The sleep quality information can be determined in a separate process than the process 2400. The sleep quality information can be determined at another time and/or by another computing system, in some implementations.

In block 2412, the computing system can return the microclimate temperature value(s) and/or the sleep quality information for the sleep session of the user. For example, the computing system can transmit instructions to present information about the user's sleep session to a computing device (e.g., mobile phone) of the user. The information can be presented in a mobile application at the computing device. The information can be presented when the user wakes up, when the user is detected as leaving the bed system, when the user opens the mobile application at their computing device, and/or a threshold amount of time after one or more of those scenarios or other scenarios after the sleep session. The presented information can include, as non-limiting examples, a graphical depiction of the microclimate temperature values throughout the sleep session of the user, a numeric and/or graphical indication of the average microclimate temperature value for the entire sleep session, and/or a numeric and/or graphical indication of the sleep quality information for the user. One or more other information may also be presented at the computing device.

In some implementations, returning the microclimate temperature value(s) and/or sleep quality information in block 2412 can include storing this information in a data store, data repository, and/or cloud-based storage system. One or more of this information can then be retrieved and used in one or more other processes described herein.

In some implementations, the process 2400 can be performed to determine microclimate temperature values for each side of the bed system, where each side of the bed system is occupied by a user. For example, the computing system can detect, based on first pressure values collected by at least one sensor of the bed system, presence of the user on a first side of the bed system. The computing system can also detect, based on second pressure values collected by the at least one sensor of the bed system, presence of a second user on a second side of the bed system. The computing system may receive, from the temperature sensors, temperature values detected at the top surface of the bed system when the user and the second user are detected on the bed system. Accordingly, the computing system can generate respective temperature profiles for the user and the second user based on applying a temperature model to the received temperature values, the temperature model having been trained to differentiate the received temperature values for respective sides of the bed system and generate a temperature profile for each side of the bed system. The computing system can also store, in the data repository described herein, the respective temperature profiles for the user and the second user for later use (e.g., by a controller of the bed system), in determining thermal settings for each side of the bed system during subsequent sleep sessions of the respective user and second user.

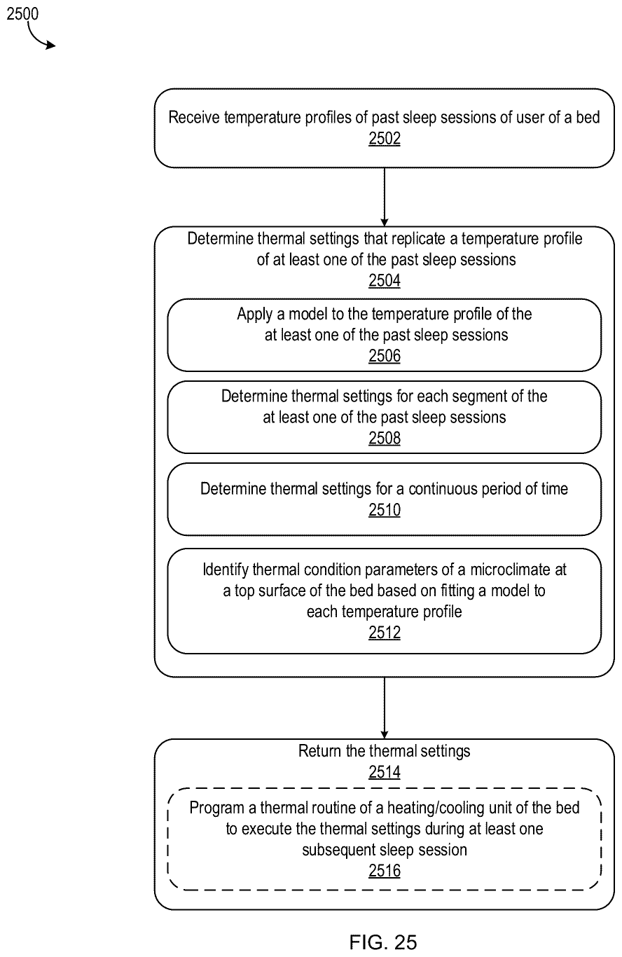

FIG. 25 is a flowchart of a process 2500 to determine thermal settings to implement during subsequent sleep sessions of a user based on processing data that corresponds to past sleep sessions of the user. The process 2500 is the same as or similar to the blocks M-P in FIG. 19A, one or more blocks in the process 2100 in FIGS. 21A-B, and/or the process 2300 in FIG. 23.

The process 2500 can be performed by the computing system 1908 described in reference to FIG. 19A. The process 2500 can also be performed by one or more other computing systems and/or devices described herein, including but not limited to a controller of the bed system, user computing devices, edge computing devices, remote computing devices, and/or cloud-based systems. For illustrative purposes, the process 2500 is described from the perspective of a computing system.

Referring to the process 2500 in FIG. 25, the computing system can receive temperature profiles of past sleep sessions of the user of a bed system (block 2502). The computing system can retrieve the temperature profiles from a data repository, data store, and/or other type of data storage system described herein. The computing system can receive the temperature profiles for a threshold quantity of past sleep sessions of the user. For example, the computing system can receive the temperature profiles for a past 3 consecutive sleep sessions, 5 consecutive sleep sessions, 7 consecutive sleep sessions, 9 consecutive sleep sessions, 14 consecutive sleep sessions, every other sleep session over a threshold amount of days (e.g., 3 days, 5 days, 7 days, 9 days, 10 days), and/or any other pattern of past sleep sessions.

The computing system can determine thermal settings that replicate a temperature profile of at least one of the past sleep sessions in block 2504. The temperature profile can include multiple temperature values detected at a top surface of the bed system throughout the at least one of the past sleep sessions of the user. For example, the temperature profile can include an aggregate microclimate temperature value of the at least one past sleep session. The temperature profile can additionally or alternatively include microclimate temperature values detected/determined at one or more predetermined intervals of time (e.g., refer to FIG. 24). Moreover, the temperature profile can include a time sequence of temperature values collected by temperature sensors of the bed system during the at least one past sleep session. The temperature values can be collected when one or more different thermal settings are activated at the bed system. The temperature values can additionally or alternatively be collected when no thermal settings are activated at the bed system.

The computing system can determine thermal settings that replicate the aggregate microclimate temperature value of the at least one past sleep session. For example, the computing system can retrieve, from the data repository described herein, a library of thermal settings that map each of the retrieved thermal settings with effects of those thermal settings on a microclimate at a top surface of the bed system. The computing system can then identify, based on the effects of the retrieve thermal settings on the microclimate, one or the retrieved thermal settings that causes the microclimate at the top surface of the bed system to achieve at least one temperature value in the temperature profile of the at least one past sleep session.

The computing system can determine thermal settings that replicate multiple of the microclimate temperature values of the at least one past sleep session. Accordingly, the computing system can determine multiple different thermal settings to achieve multiple different microclimate temperature values during the at least one past sleep session. Determining multiple different thermal settings can provide for accurate minute-by-minute (or interval of time-by-interval of time) replication of the at least one past sleep session during subsequent sleep sessions of the user. Replicating microclimate temperature values at each minute and/or interval of the past sleep session can cause the user to experience improved sleep quality over an entire subsequent sleep session.

As part of block 2504, the computing system can apply a model to the temperature profile of the at least one of the past sleep sessions (block 2506), as described throughout this disclosure.

The computing system can determine thermal settings for each segment of the at least one of the past sleep sessions in block 2508. Each segment of the sleep session can be a non-overlapping 60-minute period of time. In some implementations, each segment can be a 60-minute period of time that overlaps by some threshold amount of time (e.g., 30 seconds, 1 minute, 1.5 minutes, 2 minutes, 3 minutes, 5 minutes). In some implementations, the period of time can vary. The period of time can include, but is not limited to, 15-minutes, 30-minutes, 40-minutes, 1.5-hours, 2-hours, etc.

The computing system may additionally or alternatively determine thermal settings for a continuous period of time in block 2510. The continuous period of time can be a total amount of time that the user is on the bed system (e.g., user-presence is detected, user is detected to be sleeping) during the at least one past sleep session.

In block 2512, the computing system can identify thermal condition parameters of a microclimate at a top surface of the bed system based on fitting a model to each temperature profile. The thermal condition parameters can indicate effects of one or more different thermal settings on the microclimate at the top surface of the bed system. The computing system can then determine the thermal settings based on the identified thermal condition parameters.

Moreover, the computing system can return the thermal settings in block 2514. The computing system can store the thermal settings in the data repository described herein. The thermal settings can then be retrieved at a later time for use in one or more other process described herein. Optionally, the computing system can program a thermal routine of a heating and/or cooling unit of the bed system to execute the thermal settings during at least one subsequent sleep session (block 2516). As an illustrative example, the computing system can program the heating and/or cooling unit to execute one or more of the thermal settings based on detection of a microclimate temperature value at the bed system during the subsequent sleep session that exceeds some threshold temperature value.

Although the process 2500 is described in reference to receiving temperature profiles for the past sleep sessions of a particular user of the bed system, the process 2500 can also be performed using temperature profiles of past sleep sessions of a population of users. For example, the computing system can receive temperature profiles of past sleep sessions of a population of users that includes the user. In some implementations, the population of users may include users having similar demographics or other user information as the user. For example, the population of users can include users having a same or similar age, gender, health condition(s), geographic location, bed system, etc., as the user. The computing system can then select at least one temperature profile amongst the temperature profiles for the population of users that satisfies threshold thermal settings criteria. The threshold thermal settings criteria can indicate a threshold level of sleep quality that is associated with (e.g., has a relationship with or otherwise corresponds to) each temperature profile. If, for example, the threshold level of sleep quality is met and/or exceeded, the associated temperature profile can be selected. The computing system can determine the thermal settings that replicate the selected at least one temperature profile based on identifying relationships between temperature values of the selected at least one temperature profile and one or more predetermined thermal settings for a heating and/or cooling unit of the bed system.

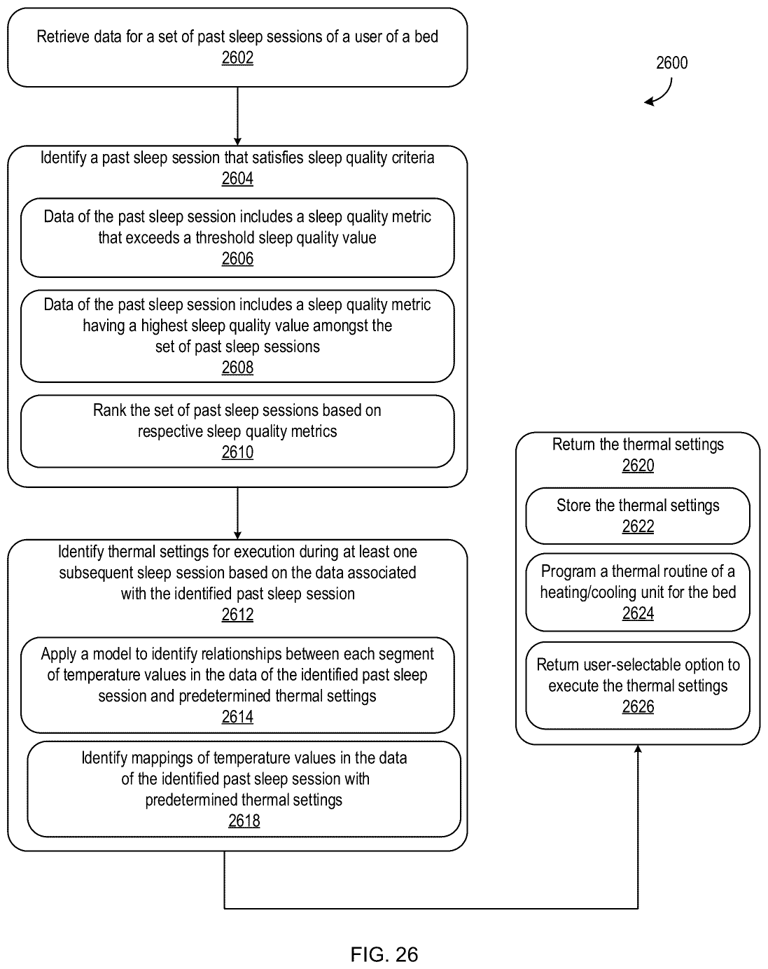

FIG. 26 is a flowchart of another process 2600 to determine thermal settings to implement during subsequent sleep sessions of a user based on processing data that corresponds to past sleep sessions of the user. The process 2600 can be the same as or similar to the blocks M-P described in FIG. 19A and/or one or more blocks in the processes 2100 and 2200 of FIGS. 21 and 22, respectively.

The process 2600 can be performed by the computing system 1908 described in reference to FIG. 19A. The process 2600 can also be performed by one or more other computing systems and/or devices described herein, including but not limited to a controller of the bed system, user computing devices, edge computing devices, remote computing devices, and/or cloud-based systems. For illustrative purposes, the process 2600 is described from the perspective of a computing system.

Referring to the process 2600, the computing system can retrieve data for a set of past sleep sessions of a user of a bed system in block 2602. The computing system can retrieve the data for a threshold quantity of past sleep sessions. The threshold quantity can vary. For example, the threshold quantity can be between 7 and 8 successive sleep sessions. As other, non-limiting examples, the threshold quantity can be 3 successive sleep sessions, 5 successive sleep sessions, 7 successive sleep sessions, 9 successive sleep sessions, etc.

In block 2604, the computing system can identify a past sleep session, based on the retrieved data, that satisfies sleep quality criteria. The sleep quality criteria can indicate temperature profile-data that results in the user experiencing a threshold level of sleep quality during the past sleep session, as described herein. The sleep quality criteria can, in some implementations, change based on a current season. The current season can include at least one of winter, spring, summer, or fall. The sleep quality criteria can additionally or alternatively change based on a circadian rhythm of the user. The sleep quality criteria can additionally or alternatively change based on an age of the user. The sleep quality criteria can additionally or alternatively change based on a day of a current week. One or more other factors may also cause the sleep quality criteria to dynamically change.

As part of identifying the past sleep session in block 2604, the computing system can determine whether data of the past sleep session includes a sleep quality metric that exceeds a threshold sleep quality value (block 2606). The sleep quality metric, as described herein, can be a numeric value indicating a sleep quality score. In some implementations, the sleep quality metric can be a user-perceived sleep quality value. For example, the user can complete a subjective survey after they wake up from a sleep session in which they indicate how they believe they slept during that sleep session. The computing system can retrieve, from the data repository and for each of the past sleep sessions, a sleep quality metric. The computing system can identify a sleep session amongst the past sleep sessions having the sleep quality metric that exceeds the threshold sleep quality value and select the temperature profile that corresponds to that identified sleep session. The sleep quality metric can be a numeric value and the threshold sleep quality value can be 90, in some implementations.

As another example, the computing system can determine whether the data of the past sleep session includes a sleep quality metric having a highest sleep quality value amongst the set of past sleep sessions (block 2608). The computing system can then select the temperature profile that corresponds to the sleep session having the highest sleep quality value.

Similarly, as another example, the computing system can rank the set of past sleep sessions based on the respective sleep quality metrics in block 2610. The computing system can then select a highest ranked temperature profile amongst the ranked temperature profiles. The temperature profiles can be ranked from highest sleep quality metric to lowest sleep quality metric. In some implementations, the computing system can identify and select past sleep sessions that are within a top threshold percentage in rankings and/or sleep quality metrics. For example, the computing system can identify the past sleep sessions that are ranked in a top 5% of all the sleep sessions. As another example, the computing system can identify the past sleep sessions that have a top 5% with respect to sleep quality metric/score values. Thermal settings can then be generated/replicated for each of those identified sleep sessions. Sometimes during runtime, those thermal settings can be randomly applied in subsequent sleep sessions.

In some implementations, the sleep session(s) can be identified using criteria that is specific to a current or upcoming season. After all, microclimate temperature profiles and settings can be different during a summer season versus a winter season.

In block 2612, the computing system can identify thermal settings for execution during at least one subsequent sleep session based on the data associated with the identified past sleep session. The computing system can determine thermal settings for each segment of the identified past sleep session. Each segment can be a 60-minute period of time. The segments can be non-overlapping periods of time during the identified past sleep session. The segments can be different in one or more other ways. For example, the segments can be different sleep stages during the sleep session.

As part of block 2612, the computing system can apply a model to identify relationships between each segment of temperature values in the data of the identified past sleep session and predetermined thermal settings (block 2614). The model can be trained to identify segments of temperature values in a temperature profile in the data for the identified past sleep session during the past sleep session and identify relationships between each of the segments of temperature values and one or more predetermined thermal settings.

As another example, the computing system can identify mappings of temperature values in the data of the identified past sleep session with predetermined thermal settings (block 2616). The computing system can retrieve, from the data repository described herein, mappings of temperature values in the data for the set of past sleep sessions to one or more predefined thermal settings. The predefined thermal settings can include high heat, medium heat, low heat, high cool, medium cool, low cool, and/or off. The computing system can then select, based on the mappings, at least one of the predefined thermal settings that corresponds to temperature values in a temperature profile in the data for the identified past sleep session.

The computing system can then return the thermal settings in block 2620. For example, the computing system can store the thermal settings as described herein (block 2622). The stored thermal settings can then be used for future use (e.g., by a controller of the bed system) to determine when to activate a heating and/or cooling unit of the bed system during the at least one subsequent sleep session. Additionally or alternatively, the computing system can program a thermal routine of a heating and/or cooling unit of the bed system according to the thermal settings (block 2624). Therefore, the heating and/or cooling unit of the bed system can execute the thermal settings during at least one subsequent sleep session of the user. Additionally or alternatively, the computing system can return a user-selectable option to execute the thermal settings during one or more subsequent sleep sessions of the user (block 2626). For example, the user-selectable option can be presented in a mobile application at a computing device (e.g., mobile phone) of the user. The user can select the option to have the thermal settings executed during at least a next sleep session of the user. The user can also select the option, or another option, to have the thermal settings executed during one or more subsequent sleep sessions. Once the user selects the option, the heating and/or cooling unit of the bed system can be pre-programmed with the thermal settings. Then, at runtime during the subsequent sleep session(s), the heating and/or cooling unit can automatically execute the thermal settings based on real-time microclimate temperatures detected at the top surface of the bed system.

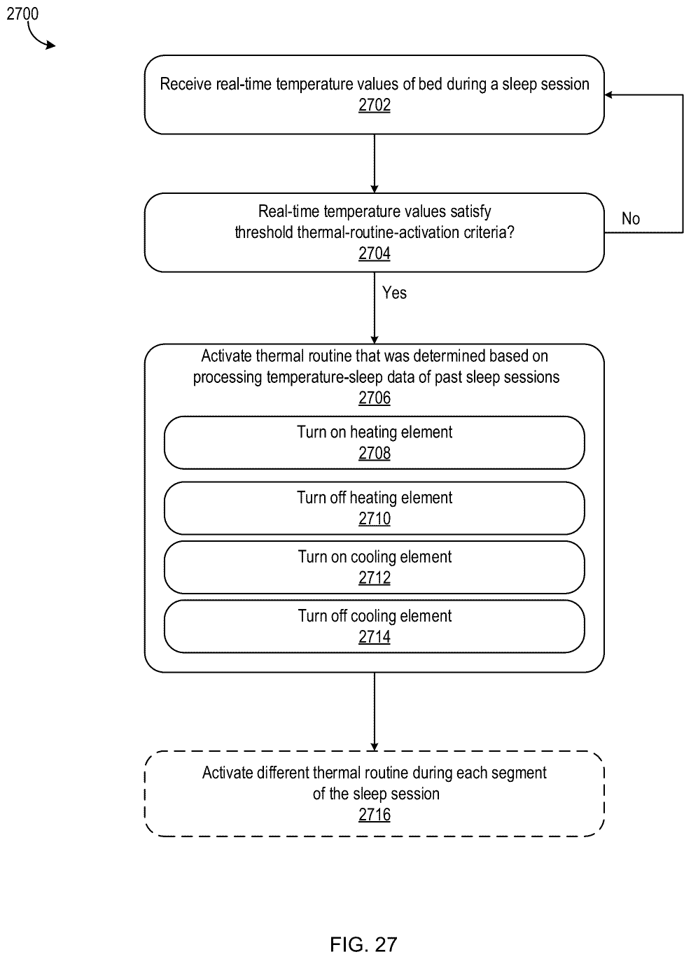

FIG. 27 is a flowchart of a process 2700 to determine, during a subsequent sleep session, when and what thermal settings to activate at a bed system to improve a user's level of sleep quality. The process 2700 is the same as or similar to blocks X-Y in FIG. 19A and/or one or more blocks in the processes 2100 and 2200 in FIGS. 21 and 22, respectively.

The process 2700 can be performed by the computing system 1908 described in reference to FIG. 19A. The process 2700 can also be performed by one or more other computing systems and/or devices described herein, including but not limited to a controller of the bed system, user computing devices, edge computing devices, remote computing devices, and/or cloud-based systems. For illustrative purposes, the process 2700 is described from the perspective of a computing system.

Referring to the process 2700 in FIG. 27, the computing system can receive real-time temperature values of the bed system (e.g., a top surface of the bed system) during a sleep session of the user (block 2702). The temperature values can be received while the user is detected as being on the bed.

The computing system can determine whether the real-time temperature values satisfy threshold thermal-routine-activation criteria in block 2704. For example, the computing system can determine whether an average of the real-time temperature values exceeds a threshold average temperature value for a target temperature profile, where the target temperature profile was used to determine the thermal settings with one or more of the above-described processes. The threshold average temperature value can be a temperature value at which the user experienced a highest level of sleep quality and/or at least a threshold level of sleep quality. Sometimes, the computing system can determine whether an average of the real-time temperature values is less than a threshold average temperature value for the target temperature profile in block 2704 (e.g., depending on whether the thermal routine is being activated in a heating mode versus a cooling mode). In some implementations, the criteria can vary based on one or more factors. The factors can include, but are not limited to, a current season (e.g., winter, spring, summer, or fall), a circadian rhythm of the user, an age of the user, a day of the week, etc.

If the criteria is not satisfied, the computing system can return to block 2702 and continue to receive real-time temperature values from the bed system during the user's sleep session.

If the criteria is satisfied, the computing system can activate a thermal routine that was determined based on processing temperature-sleep data of past sleep sessions of the user (block 2706).

For example, the computing system can turn on a heating element (block 2708). The heating element can be turned on to high, medium, or low settings, based on thermal settings that were determined using the abovementioned processes. The computing system can turn on the heating element of the heating or cooling unit to a setting defined by the thermal settings until at least one temperature value in a target temperature profile is detected, by the temperature sensors, at the top surface of the bed system. The target temperature profile can be used by the computing system in the abovementioned processes to generate the thermal settings.

The computing system can turn off the heating element (block 2710). For example, the computing system can turn off the heating element of the heating or cooling unit while the at least one temperature value in the target temperature profile is detected, by the temperature sensors, at the top surface of the bed system.

The computing system can turn on a cooling element (block 2112). The cooling element can be turned on to high, medium, or low settings, based on thermal settings that were determined using the abovementioned processes. For example, the computing system can turn on the cooling element of the heating or cooling unit until the at least one temperature value in the target temperature profile is detected, by the temperature sensors, at the top surface of the bed system.

The computing system can turn off the cooling element (block 2114). For example, the computing system can turn off the cooling element of the heating or cooling unit while the at least one temperature value in the target temperature profile is detected, by the temperature sensors, at the top surface of the bed system.

Optionally, the computing system may activate a different thermal routine during each segment of the sleep session of the user (block 2716). For example, the sleep session can include one or more segments. The threshold thermal-routine-activation criteria can be different for each segment of the sleep session. The computing system can then activate the thermal routine during each segment based on a determination of whether the respective threshold-thermal-routine-activation criteria for the segment is satisfied.

As an illustrative example, the user may fall asleep faster when the microclimate of their bed system is above a first threshold temperature value. Therefore, during a first segment of the sleep session, the computing system can determine whether real-time temperature values indicate that the microclimate of the bed system is less than the first threshold temperature value. If so, then the computing system can activate a thermal routine that causes the heating element to turn on until the first threshold temperature value is reached or maintained for a threshold amount of time and/or until the user falls asleep and/or enters a next segment of the sleep session. Then, midway through the sleep session, the user may experience a highest level of sleep quality when the microclimate is below a second threshold temperature value. Accordingly, the computing system can activate a thermal routine that causes the cooling element to turn on when the microclimate is detected to exceed the second threshold temperature value.

What is claimed is:

1. A bed system for improving sleep quality of a user by adjusting a sleep environment, the bed system comprising:
a controller configured to:
retrieve, from a data repository, data for a set of past sleep sessions of a user;
identify, based on the data, a past sleep session in the set of past sleep sessions that satisfies sleep quality criteria;
determine thermal settings for the bed system based on the data associated with the identified past sleep session of the user; and
apply the thermal settings to the bed system for execution during at least one subsequent sleep session of the user.

2. The bed system of claim 1, wherein the set of past sleep sessions include multiple past sleep sessions of the user.

3. The bed system of claim 1, wherein identifying the past sleep session in the set comprises determining that a sleep quality value in the data for the identified sleep session is greater than sleep quality values in the data for other past sleep sessions in the set.

4. The bed system of claim 1, wherein identifying the past sleep session in the set comprises determining that a sleep quality value in the data for the identified past sleep session exceeds a threshold sleep quality value.

5. The bed system of claim 1, wherein the data for the set of past sleep sessions includes a temperature profile for each of the past sleep sessions, wherein the temperature profile is a time sequence of temperature values collected by temperature sensors at a top surface of the bed system of the user throughout the past sleep session.

6. The bed system of claim 5, wherein determining the thermal settings for the bed system comprises applying a model to a temperature profile of the identified past sleep session, the model having been trained to identify segments of temperature values in the temperature profile and identify relationships between each of the segments of temperature values and one or more predetermined thermal settings.

7. The bed system of claim 1, wherein applying the thermal settings to the bed system comprises generating instructions to activate a thermal routine at a heating or cooling unit of the bed system during the at least one subsequent sleep session of the user to adjust a microclimate at a top surface of the bed system to at least one temperature value in the data for the identified past sleep session.

8. The bed system of claim 1, wherein determining the thermal settings for the bed system includes determining the thermal settings for each segment of the identified past sleep session.

9. The bed system of claim 8, wherein each segment of the identified past sleep session is a 60-minute period of time, wherein the segments are non-overlapping periods of time during the identified past sleep session.

10. The bed system of claim 1, wherein identifying, based on the data, the past sleep session in the set of past sleep sessions that satisfies sleep quality criteria comprises identifying the past sleep session in the set having a sleep quality metric in the data for the past sleep session that exceeds a threshold sleep quality value.

11. The bed system of claim 10, wherein the sleep quality metric in the data for the past sleep session is a numeric value indicating a sleep quality score and the threshold sleep quality value is 90.

12. The bed system of claim 1, wherein identifying, based on the data, the past sleep session in the set of past sleep sessions that satisfies sleep quality criteria comprises:
ranking the past sleep sessions in the set from highest sleep quality metric to lowest sleep quality metric, wherein the data for each of the past sleep sessions in the set includes a sleep quality metric corresponding to the past sleep session; and
selecting the past sleep session that is ranked the highest among the ranked past sleep sessions.

13. A bed system for improving sleep quality of a user by adjusting a sleep environment, the bed system comprising:
a controller configured to:
receive, from at least one temperature sensor of the bed system, temperature data collected during a sleep session of a user of the bed system;
determine a microclimate temperature value for each predetermined interval of time during the sleep session based on applying a model to the temperature data;
determine a microclimate temperature value for the sleep session based on aggregating the microclimate temperature values for the predetermined intervals of time during the sleep session; and
return at least one of (i) the microclimate temperature values for the predetermined intervals of time during the sleep session or (ii) the microclimate temperature value for the sleep session.

14. The bed system of claim 13, wherein the at least one temperature sensor is configured to a sensor strip, the sensor strip removably attached to a top surface of the bed system on which the user rests.

15. The bed system of claim 14, wherein the at least one temperature sensor includes temperature sensors linearly arranged along the sensor strip, wherein the 5 temperature sensors are equally spaced apart by a threshold distance along the sensor strip.

16. The bed system of claim 15, wherein the bed system is a queen-sized bed and the threshold distance is 5.5 inches.

17. The bed system of claim 15, wherein the bed system is a king-sized bed and the threshold distance is 6.5 inches.

18. The bed system of claim 13, wherein the model was trained to approximate, based on the temperature data, a microclimate temperature value for each of the predetermined time intervals during the sleep session.

19. The bed system of claim 18, wherein the predetermined time intervals are one-minute segments during the sleep session.

20. A computer-readable medium storing instructions that, when executed by one or more processors, cause the processors to perform operations comprising:
receiving, from at least one temperature sensor of a bed system, temperature data collected during a sleep session of a user of the bed system;
determining a microclimate temperature value for each predetermined interval of time during the sleep session based on applying a model to the temperature data;
determining a microclimate temperature value for the sleep session based on aggregating the microclimate temperature values for the predetermined intervals of time during the sleep session; and
returning at least one of (i) the microclimate temperature values for the predetermined intervals of time during the sleep session or (ii) the microclimate temperature value for the sleep session.
